(19) United States
(12) Patent Application Publication
(10) Pub. No.: US 2026/0247786 A1
(43) Pub. Date: Aug. 20, 2026

(54) ORGANIC COMPOUNDS AND ORGANIC LIGHT EMITTING DIODE COMPRISING THE SAME

(71) Applicant: Material Science Co., Ltd., Hwaseong-si (KR)

(72) Inventors: Ha Ni JEON, Hwaseong-si (KR); Tae Wan LEE, Hwaseong-si (KR)

(21) Appl. No.: 19/539,209

(22) Filed: Feb. 13, 2026

(30) Foreign Application Priority Data
Feb. 14, 2025 (KR) 10-2025-0019444
Mar. 6, 2025 (KR) 10-2025-0028961
Feb. 11, 2026 (KR) 10-2026-0027878

(51) Int. Cl. H10K 50/11; H10K 85/60

(52) U.S. Cl. CPC H10K 50/11; H10K 85/633

(57) ABSTRACT

The organic compound represented by Chemical Formula A according to the present disclosure can exhibit excellent hole transport properties. In addition, the hole transport layer or hole transport auxiliary layer of the organic light-emitting diode according to the present disclosure includes an organic compound represented by Chemical Formula A according to the present disclosure, thereby improving the driving voltage, efficiency, and lifetime characteristics of the organic light-emitting diode.

### CROSS-REFERENCE TO RELATED APPLICATION

This application claims priority from Korean Patent Applications No. 10-2025-0019444 filed on Feb. 14, 2025, No. 10-2025-0028961 filed on Mar. 6, 2025, and No. 10-2026-0027878 filed on Feb. 11, 2026 in the Korean Intellectual Property Office, and all the benefits accruing therefrom under 35 U.S.C. 119, the contents of which in its entirety are herein incorporated by reference.

### TECHNICAL FIELD

The present disclosure relates to an organic compound and an organic light-emitting diode comprising the same.

### BACKGROUND

Organic light-emitting diodes (OLEDs) have been actively developed and commercialized as a light source for flat panel displays such as wall-mountable televisions, backlights for displays, lighting devices, and signboards, because they have simplified structure, various advantages in manufacturing processes, high luminance, excellent viewing angle characteristics, a fast response speed, and a low driving voltage, compared to other flat panel display devices such as conventional liquid crystal displays (LCDs), plasma display panels (PDPs), and field emission displays (FEDs).

The OLED includes two electrodes and an organic layer disposed between the two electrodes. The OLED is a device that operates on the principle that electrons and holes are injected into an emitting layer from the two electrodes, respectively, and recombine within the emitting layer to generate excitons, and light is generated when the generated excitons transition from an excited state to a ground state.

The OLED may include at least one emitting layer. In general, an OLED having a plurality of emitting layers may include emitting layers that emit light having different peak wavelengths, thereby enabling a specific color to be realized through a combination of light having different peak wavelengths.

These OLEDs may be categorized into a bottom-emitting structure and a top-emitting structure. The bottom-emitting device uses a reflective second electrode (cathode) to emit light generated in the emitting layer toward a translucent or transparent first electrode (anode). In contrast, the top-emitting device uses a reflective first electrode to emit light generated in the emitting layer and reflected by the first electrode toward a transparent second electrode in the direction of a driving thin-film transistor.

### RELATED ART DOCUMENT

### [Patent Document]

(Patent Document 1) KR 2020-0122116 A
(Patent Document 2) WO 2021/070964 A1
(Patent Document 3) US 2018-0219157 A1

### SUMMARY

An object of the present disclosure is to provide a novel organic compound and an organic light-emitting diode comprising the same.

Embodiments according to the present disclosure may be used to achieve other problems that are not specifically mentioned, in addition to the above problems.

The present disclosure is not limited to the objects described above, and other objects and advantages of the present disclosure that are not mentioned herein will be understood from the following description and will be more clearly understood from the embodiments of the present disclosure. Furthermore, it will be readily apparent that the objects and advantages of the present disclosure may be realized by means and combinations thereof set forth in the claims.

According to an embodiment of the present disclosure, an organic compound represented by the following Chemical Formula A may be provided. The definition of the following Chemical Formula A is identical to that described in the present specification and claims:

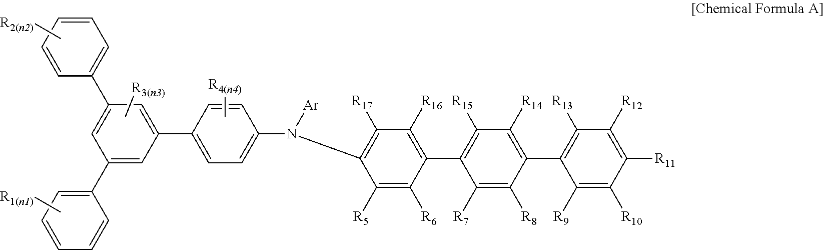

wherein in the chemical formula A,
Ar is one selected from the structures of M2, M3, M4, M11, M12, M13, M14, M15, M16, M17, M18, M19, M20, M21, M22, M23, M24, M25, M26, M27, M28, M29, M30 and M31,
R₁ to R₁₇ are identical to or different from each other, and each of R₁ to R₁₇ independently represents one selected from the group consisting of hydrogen, deuterium, a substituted or unsubstituted alkyl group having 1 to 30 carbon atoms, a substituted or unsubstituted aryl group having 6 to 30 carbon atoms, and a substituted or unsubstituted heteroaryl group having 3 to 60 carbon atoms,
n1 and n2 are identical to or different from each other, and are each independently an integer from 0 to 5,
n3 is an integer from 0 to 3,
n4 is an integer from 0 to 4, and
when Ar and R₁ to R₁₇ are substituted, the substituents are identical to or different from each other, and each substituent may be one or more selected from the group consisting of deuterium, a cyano group, a trifluoromethyl group, a nitro group, a halogen group, a hydroxy group, a trimethylsilyl (TMS) group, an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 3 to 60 carbon atoms, a heteroarylalkyl group having 4 to 60 carbon atoms, an amino group, an alkylamino group having 1 to 30 carbon atoms, an arylalkylamino group having 7 to 30 carbon atoms, an arylamino group having 6 to 30 carbon atoms, a heteroarylamino group having 3 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms; and when a plurality of substituents are present, the substituents are identical to or different from each other.

The organic compound represented by Chemical Formula A according to the present disclosure can exhibit excellent hole transport properties.

In addition, one or more of a hole transport layer and a hole transport auxiliary layer of an organic light-emitting diode according to the present disclosure include the organic compound represented by Chemical Formula A according to the present disclosure, thereby improving driving voltage, external quantum efficiency, and lifetime characteristics of the organic light-emitting diode.

Further, when the organic compound represented by Chemical Formula A according to the present disclosure is used as a hole transport auxiliary layer material, the organic compound can have an energy level suitable for serving as the hole transport auxiliary layer, which transports holes from a hole transport layer to an emitting layer and blocks electrons coming from an emitting layer.

Further, in the organic light-emitting diode according to the present disclosure, a hole transport layer and/or a hole transport auxiliary layer including the organic compound represented by Chemical Formula A according to the present disclosure can achieve excellent implementation of target color coordinates of an emitting layer, even when combined with an emitting layer of any color.

The effects of the present disclosure are not limited to those described above, and other effects that are not mentioned will be clearly understood by those skilled in the art from the entirety of the present specification. The above effects and additional effects will be described in detail below.

### DETAILED DESCRIPTION

The aforementioned objects, features, and advantages will be described in detail below, and accordingly, those skilled in the art to which the present disclosure pertains will be able to easily implement the technical spirit of the present disclosure.

In describing this specification, the detailed descriptions of related known technologies will be omitted if they are deemed to unnecessarily obscure the gist of the present disclosure.

As used herein, it is to be understood that when the terms such as “comprise,” “have,” “consist of,” “disposed,” “provided,” etc. are used with respect to components, additional components may be present, unless the term “only” is used. Also, it is to be understood that when a component is described in the singular, it is intended to include the plural, unless expressly stated otherwise.

In interpreting the components in the present specification, it is to be understood that the ranges include allowable tolerances even if not explicitly stated.

As used herein, when any configuration is described as being disposed “on (or under)” a component or “on an upper portion (or lower portion)” of a component, it may mean not only that any configuration is disposed directly contacting the top (or bottom) surface of the component, but also that another configuration may be interposed between the component and any configuration disposed on (or under) the component.

As used herein, the term “halogen group” includes fluorine, chlorine, bromine, and iodine.

As used herein, the term “alkyl group” refers to both linear alkyl radicals and branched alkyl radicals. Unless otherwise specified, the alkyl group has 1 to 30 carbon atoms and may include, but is not limited to, methyl, ethyl, propyl, isopropyl, butyl, sec-butyl, isobutyl, tert-butyl, pentyl, isoamyl, hexyl, and the like. Additionally, the alkyl group may be optionally substituted.

As used herein, the term “cycloalkyl group” refers to cyclic alkyl radicals. Unless otherwise specified, the cycloalkyl group has 3 to 20 carbon atoms and may include, but is not limited to, cyclopropyl, cyclobutyl, cyclopentyl, cyclohexyl, norbornyl, adamantyl, and the like. Additionally, the cycloalkyl group may be optionally substituted.

As used herein, the term “alkenyl group” refers to both linear alkenyl radicals and branched alkenyl radicals having one or more carbon-carbon double bonds. Unless otherwise specified, the alkenyl group has 2 to 30 carbon atoms and may include, but is not limited to, vinyl, allyl, isopropenyl, 2-butenyl, and the like. Additionally, the alkenyl group may be optionally substituted.

As used herein, the term “cycloalkenyl group” refers to a cyclic alkenyl radical. Unless otherwise specified, the cycloalkenyl group has 3 to 20 carbon atoms. Additionally, the cycloalkenyl group may be optionally substituted.

As used herein, the term “alkynyl group” refers to both linear alkynyl radicals and branched alkynyl radicals having one or more carbon-carbon triple bonds. Unless otherwise specified, the alkynyl group has 2 to 30 carbon atoms and may include, but is not limited to, ethynyl, 2-propynyl, and the like. Additionally, the alkynyl group may be optionally substituted.

As used herein, the term “cycloalkynyl group” refers to a cyclic alkynyl radical. Unless otherwise specified, the cycloalkynyl group has 3 to 20 carbon atoms. Additionally, the cycloalkynyl group may be optionally substituted.

As used herein, the terms “aralkyl group” or “arylalkyl group” is used interchangeably, and refers to an alkyl group having an aromatic group as a substituent. Additionally, the aralkyl group (arylalkyl group) may be optionally substituted.

As used herein, the terms “aryl group” and “aromatic group” are used interchangeably, and the aryl group includes both monocyclic and polycyclic groups. A polycyclic ring may include a “fused ring,” in which two or more rings are formed by sharing two carbon atoms between two adjacent rings. It may also include a form in which two or more rings are simply linked or fused to each other. Unless otherwise specified, the aryl group has 6 to 30 carbon atoms, and may include, but is not limited to, phenyl, naphthyl, phenanthryl, anthryl, fluorenyl, dimethylfluorenyl, diphenylfluorenyl, spirobifluorenyl, and the like. Additionally, the aryl group may be optionally substituted.

As used herein, the terms “heteroaryl group” and “heteroaromatic group” are used interchangeably, and the heteroaryl group includes both monocyclic and polycyclic groups. A polycyclic ring may include a “fused ring,” in which two or more rings are formed by sharing two carbon atoms or heteroatoms between two adjacent rings. It may also include a form in which two or more rings are simply linked or fused to each other. Unless otherwise specified, the heteroaryl group has 3 to 60 carbon atoms, wherein one or more carbon atoms in the ring are replaced with heteroatoms such as oxygen (O), nitrogen (N), sulfur (S), or selenium (Se). The heteroaryl group may include, but is not limited to, 6-membered monocyclic rings such as pyridyl, pyrazinyl, pyrimidinyl, pyridazinyl, and triazinyl, polycyclic rings such as phenoxathinyl, indolizinyl, indolyl, purinyl, quinolyl, isoquinolyl, benzoxazolyl, benzothiazolyl, dibenzoxazolyl, dibenzothiazolyl, benzoimidazolyl, benzofuranyl, dibenzofuranyl, benzothiophenyl, dibenzothiophenyl, carbazolyl, phenylcarbazolyl, 9-phenylcarbazolyl, and 2-furanyl, N-imidazolyl, 2-isoxazolyl, 2-pyridinyl, 2-pyrimidinyl, and the like. Additionally, the heteroaryl group may be optionally substituted.

As used herein, the term “heterocyclic group” refers to a group in which one or more carbon atoms constituting an aryl group, a cycloalkyl group, a cycloalkenyl group, a cycloalkynyl group, an arylalkyl group, an arylamino group, or the like are replaced with heteroatoms such as oxygen (O), nitrogen (N), or sulfur (S). With reference to the above definition, the heterocyclic group includes a heteroaryl group, a heterocycloalkyl group, a heterocycloalkenyl group, a heterocycloalkynyl group, a heteroarylalkyl group, a heteroarylamino group, and the like. Additionally, the heterocyclic group may be optionally substituted.

As used herein, the term “carbon ring” refers to a term that, unless otherwise specified, includes both an alicyclic ring group, namely “cycloalkyl group,” “cycloalkenyl group,” and “cycloalkynyl group,” and an aromatic ring group, namely “aryl group (aromatic group).”

As used herein, the terms “heteroalkyl group,” “heteroalkenyl group,” “heteroalkynyl group,” and “heteroarylalkyl group,” refer to a group in which one or more carbon atoms constituting the group are replaced with heteroatoms such as oxygen (O), nitrogen (N), or sulfur (S). Additionally, the heteroalkyl group, heteroalkenyl group, heteroalkynyl group, and heteroarylalkyl group may be optionally substituted.

As used herein, the terms “alkylamino group,” “arylalkylamino group,” “arylamino group,” and “heteroarylamino group” refer to an amino group (or amine group) substituted with the alkyl group, arylalkyl group, aryl group, or heteroaryl group, and are intended to include all of primary, secondary, and tertiary amino groups (or amine groups). Additionally, the alkylamino group, arylalkylamino group, arylamino group, and heteroarylamino group may be optionally substituted.

As used herein, the terms “alkylsilyl group,” “arylsilyl group,” “alkoxy group,” “aryloxy group,” “alkylthio group,” and “arylthio group” refer to a silyl group, an oxy group, and a thio group substituted with the alkyl group and the aryl group, respectively. Additionally, the alkylsilyl group, arylsilyl group, alkoxy group, aryloxy group, alkylthio group, and arylthio group may be optionally substituted.

As used herein, the terms “arylene group,” “arylalkylene group,” “heteroarylene group,” and “heteroarylalkylene group” mean that each of the aryl group, arylalkyl group, heteroaryl group, and heteroarylalkyl group is a divalent substituent further substituted with one group. Additionally, the arylene group, arylalkylene group, heteroarylene group, and heteroarylalkylene group may be optionally substituted.

As used herein, the term “substitution” means that a hydrogen (H) atom bonded to a carbon atom, a nitrogen atom, or the like in a compound of the present disclosure is replaced with a substituent other than hydrogen. When a plurality of substituents are present, each substituent may be identical to or different from each other.

The substituents may each independently be selected from the group consisting of deuterium, a cyano group, a trifluoromethyl group, a nitro group, a halogen group, a hydroxy group, a trimethylsilyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 3 to 60 carbon atoms, a heteroarylalkyl group having 4 to 60 carbon atoms, an amino group, an alkylamino group having 1 to 30 carbon atoms, an arylalkylamino group having 7 to 30 carbon atoms, an arylamino group having 6 to 30 carbon atoms, a heteroarylamino group having 3 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms.

Unless otherwise specifically limited in this specification, the position to be substituted is not limited as long as it is a position where a hydrogen atom may be substituted, i.e., a position where a substituent may be substituted. When two or more substituents are present, the substituents may be identical to or different from each other.

Each entity and substituent defined herein may be identical to or different from each other, unless otherwise specified.

Unless otherwise specified, the units used herein are based on weight (wt). For example, when “%” is stated, it is to be construed as weight percent (wt %).

Hereinafter, an organic compound according to the present disclosure and an organic light-emitting diode comprising the same will be described in detail.

The organic compound according to the present disclosure may be represented by the following Chemical Formula A:

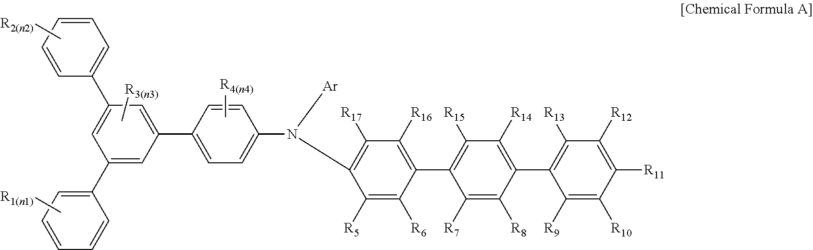

wherein in the chemical formula A,
Ar is a substituted or unsubstituted aryl group having 6 to 30 carbon atoms,
R₁ to R₁₇ are identical to or different from each other, and each of R₁ to R₁₇ independently represents one selected from the group consisting of hydrogen, deuterium, a substituted or unsubstituted alkyl group having 1 to 30 carbon atoms, a substituted or unsubstituted aryl group having 6 to 30 carbon atoms, and a substituted or unsubstituted heteroaryl group having 3 to 60 carbon atoms,
n1 and n2 are identical to or different from each other, and are each independently an integer from 0 to 5,
n3 is an integer from 0 to 3,
n4 is an integer from 0 to 4, and
when Ar and R₁ to R₁₇ are substituted, the substituents are identical to or different from each other, and each substituent may be one or more selected from the group consisting of deuterium, a cyano group, a trifluoromethyl group, a nitro group, a halogen group, a hydroxy group, a trimethylsilyl (TMS) group, an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 3 to 60 carbon atoms, a heteroarylalkyl group having 4 to 60 carbon atoms, an amino group, an alkylamino group having 1 to 30 carbon atoms, an arylalkylamino group having 7 to 30 carbon atoms, an arylamino group having 6 to 30 carbon atoms, a heteroarylamino group having 3 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms; and when a plurality of substituents are present, the substituents are identical to or different from each other.

According to an embodiment of the present disclosure, Ar may be a substituted or unsubstituted aryl group having 6 to 25, 6 to 15, 6 to 12, or 6 to 10 carbon atoms.

According to an embodiment of the present disclosure, Ar may be a substituted or unsubstituted phenyl group, a substituted or unsubstituted biphenyl group, a substituted or unsubstituted terphenyl group, a substituted or unsubstituted naphthyl group, a substituted or unsubstituted anthryl group, a substituted or unsubstituted fluorenyl group, a substituted or unsubstituted dimethylfluorenyl group, a substituted or unsubstituted diphenylfluorenyl group, a substituted or unsubstituted spirobifluorenyl group, a substituted or unsubstituted naphthyl-phenyl group, a substituted or unsubstituted dimethylfluorenyl-phenyl group, or a substituted or unsubstituted phenanthryl-phenyl group.

According to an embodiment of the present disclosure, Ar may be a phenyl group unsubstituted or substituted with deuterium, a biphenyl group unsubstituted or substituted with deuterium, a naphthyl group unsubstituted or substituted with deuterium, a dimethylfluorenyl group unsubstituted or substituted with deuterium, a naphthyl-phenyl group unsubstituted or substituted with deuterium, a dimethylfluorenyl-phenyl group unsubstituted or substituted with deuterium, or a phenanthryl-phenyl group unsubstituted or substituted with deuterium.

According to an embodiment of the present invention, Ar may be a structure in which a monocyclic or condensed ring is linked by a phenylene group(linker). For example, the monocyclic ring may be a phenyl group, and the condensed ring may be a dimethylfluorenyl group, a naphthyl group, or a phenanthryl group.

According to an embodiment of the present disclosure, Ar may be one of the substituents represented by M1 to M31 below. In M1 to M31 below, * indicates a bonding position. For example, the Ar may be one of the substituent structures represented by the following M2, M3, M4, M11, M12, M13, M14, M15, M16, M17, M18, M19, M20, M21, M22, M23, M24, M25, M26, M27, M28, M29, M30, and M31.

According to an embodiment of the present disclosure, in M1 to M31 below, Dn represents the number of deuterium atoms substituted in the structure, wherein n, which represents the number of deuterium atoms, is an integer of 0 or more.

According to an embodiment of the present disclosure, in Formula M1, n is an integer selected from 0 to 5, in Formulae M2 to M4, n is an integer selected from 0 to 9, in Formulas M5 and M6, n is an integer selected from 0 to 7, in Formulas M7 to M10, n is an integer selected from 0 to 13, in Formulas M11 to M16, n is an integer selected from 0 to 11, in Formulas M17 to M28, n is an integer selected from 0 to 17, and in Formulas M29 to M31, n is an integer selected from 0 to 13.

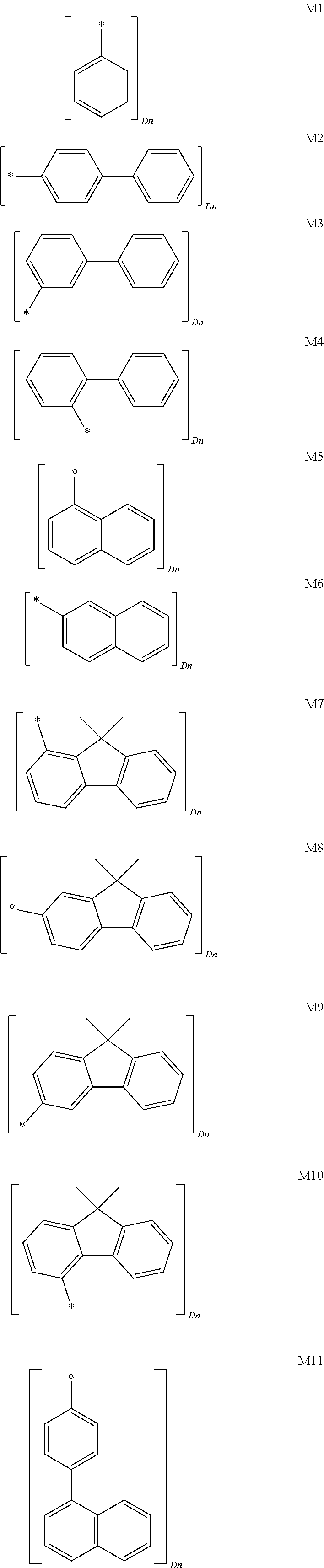

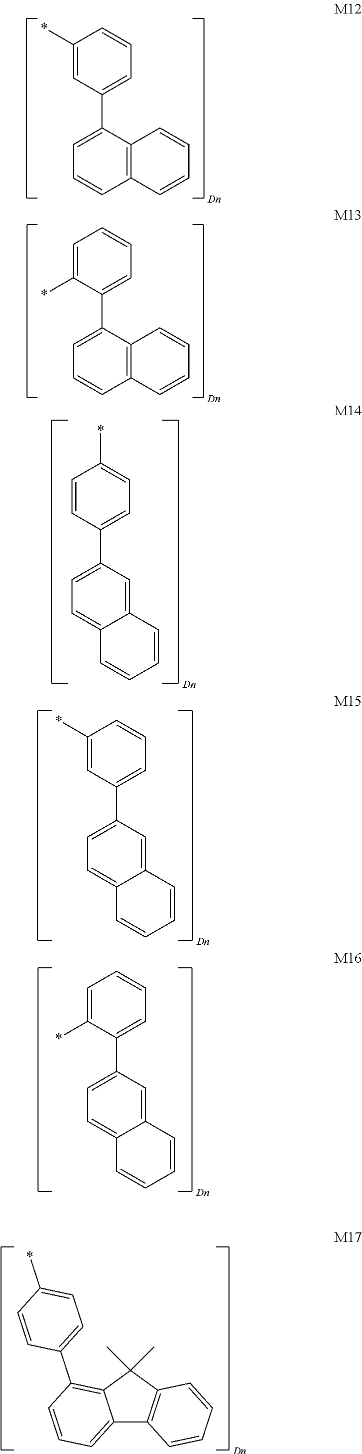

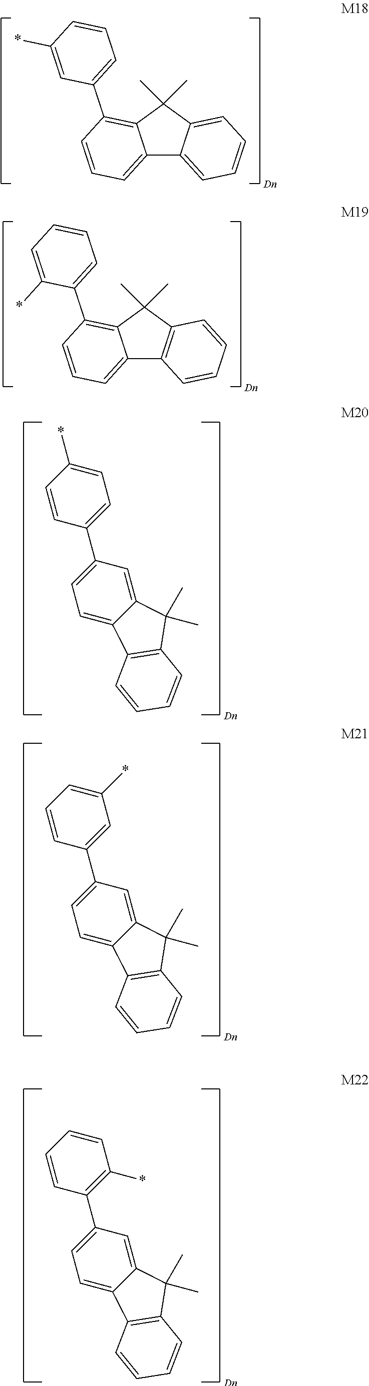

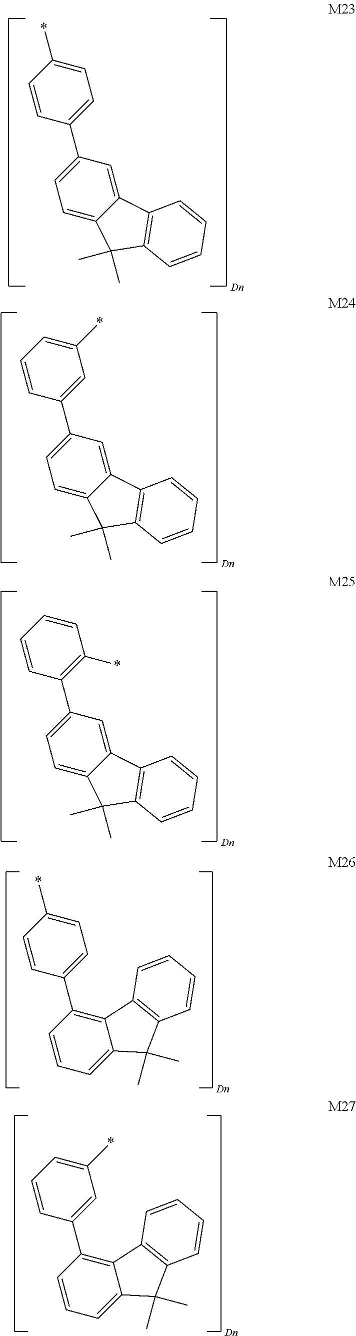

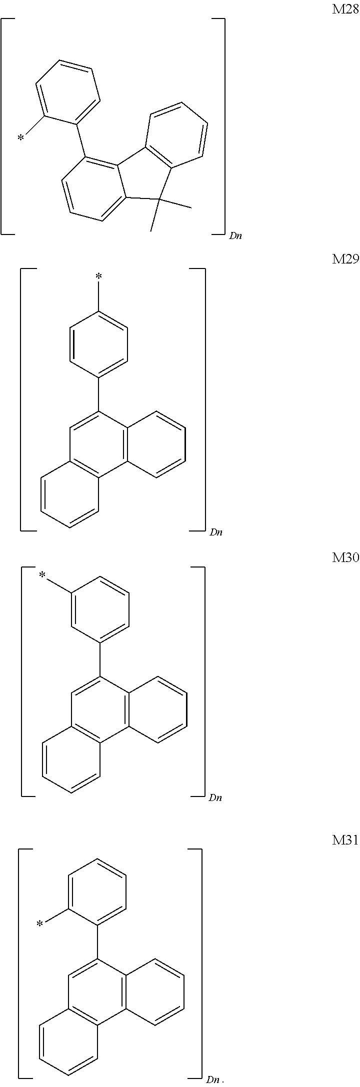

According to an embodiment of the present disclosure, R₁ to R₁₇ are identical to or different from each other, and may each independently be hydrogen or deuterium.

According to an embodiment of the present disclosure, the compound represented by Chemical Formula A may be selected from the group consisting of the following compounds, but is not limited thereto:

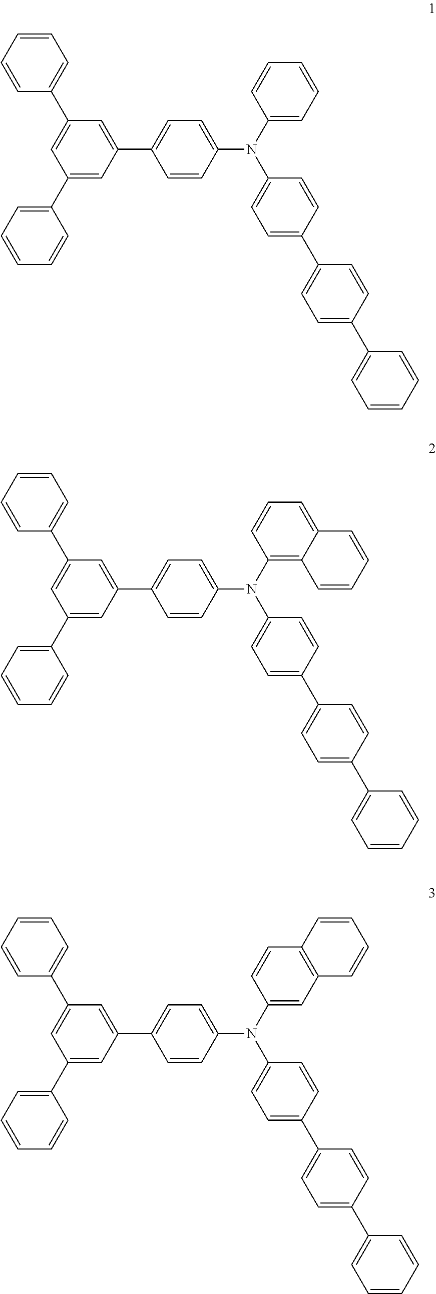 [SMILES: c1ccc(-c2ccc(-c3ccc(N(c4ccccc4)c4ccc(-c5cc(-c6ccccc6)cc(-c6ccccc6)c5)cc4)cc3)cc2)cc1 ; c1ccc(-c2ccc(-c3ccc(N(c4ccc(-c5cc(-c6ccccc6)cc(-c6ccccc6)c5)cc4)c4cccc5ccccc45)cc3)cc2)cc1 ; c1ccc(-c2ccc(-c3ccc(N(c4ccc(-c5cc(-c6ccccc6)cc(-c6ccccc6)c5)cc4)c4ccc5ccccc5c4)cc3)cc2)cc1]

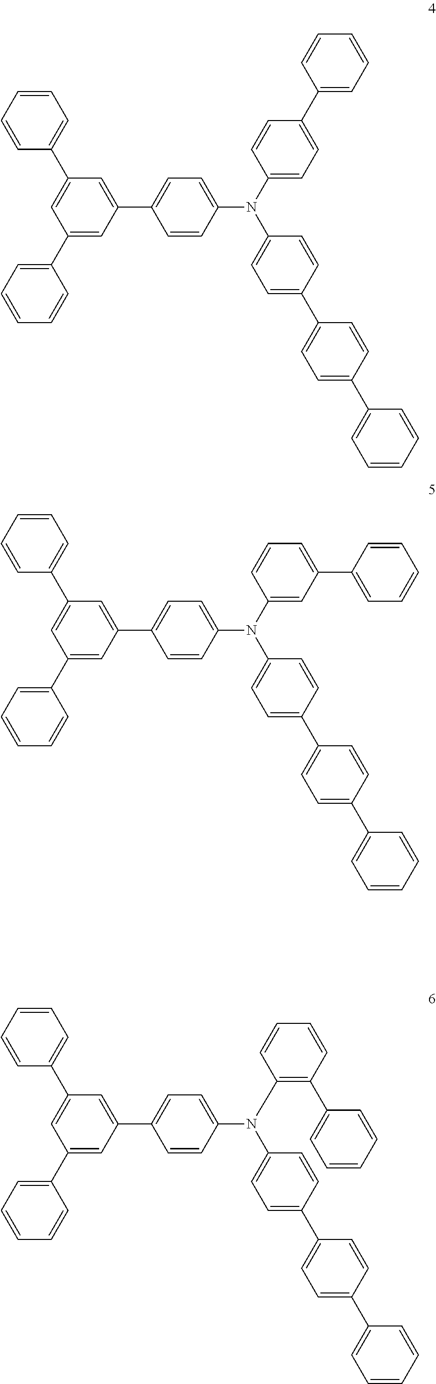 [SMILES: c1ccc(-c2ccc(-c3ccc(N(c4ccc(-c5ccccc5)cc4)c4ccc(-c5cc(-c6ccccc6)cc(-c6ccccc6)c5)cc4)cc3)cc2)cc1 ; c1ccc(-c2ccc(-c3ccc(N(c4ccc(-c5cc(-c6ccccc6)cc(-c6ccccc6)c5)cc4)c4cccc(-c5ccccc5)c4)cc3)cc2)cc1 ; c1ccc(-c2ccc(-c3ccc(N(c4ccc(-c5cc(-c6ccccc6)cc(-c6ccccc6)c5)cc4)c4ccccc4-c4ccccc4)cc3)cc2)cc1]

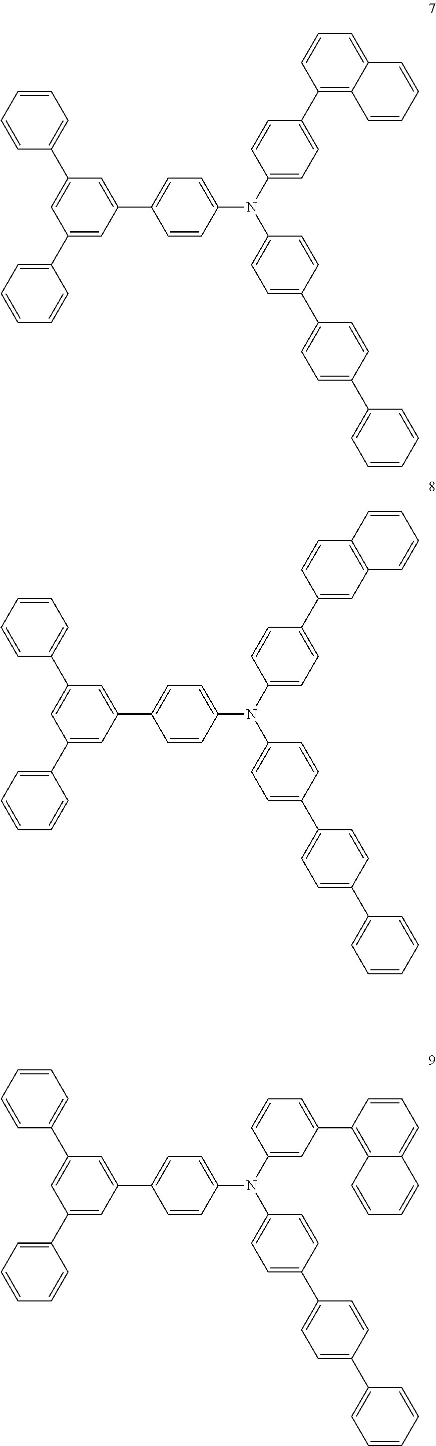

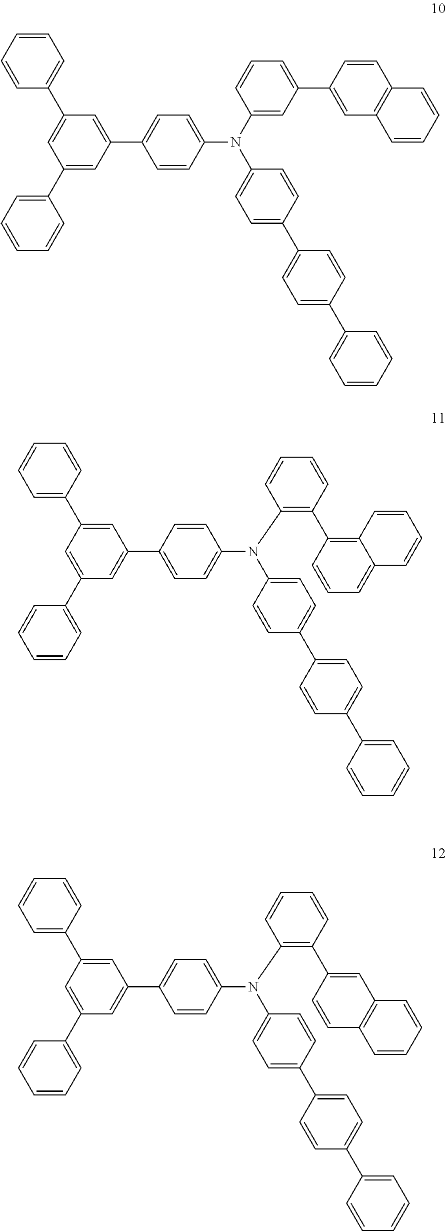 [SMILES: c1ccc(-c2ccc(-c3ccc(N(c4ccc(-c5cc(-c6ccccc6)cc(-c6ccccc6)c5)cc4)c4cccc(-c5ccc6ccccc6c5)c4)cc3)cc2)cc1 ; c1ccc(-c2ccc(-c3ccc(N(c4ccc(-c5cc(-c6ccccc6)cc(-c6ccccc6)c5)cc4)c4ccccc4-c4cccc5ccccc45)cc3)cc2)cc1 ; c1ccc(-c2ccc(-c3ccc(N(c4ccc(-c5cc(-c6ccccc6)cc(-c6ccccc6)c5)cc4)c4ccccc4-c4ccc5ccccc5c4)cc3)cc2)cc1]

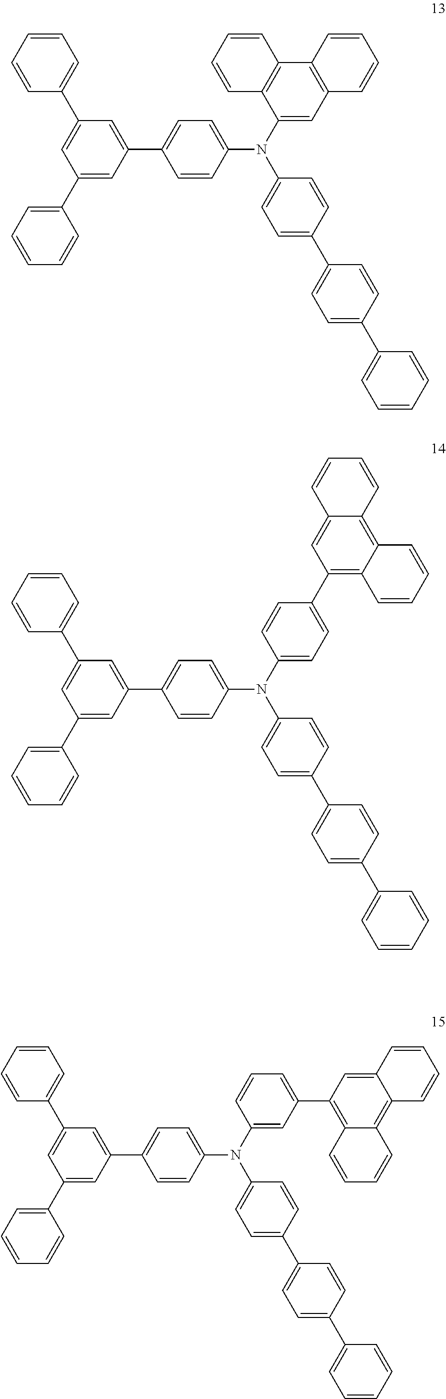 [SMILES: c1ccc(-c2ccc(-c3ccc(N(c4ccc(-c5cc(-c6ccccc6)cc(-c6ccccc6)c5)cc4)c4cc5ccccc5c5ccccc45)cc3)cc2)cc1 ; c1ccc(-c2ccc(-c3ccc(N(c4ccc(-c5cc(-c6ccccc6)cc(-c6ccccc6)c5)cc4)c4ccc(-c5cc6ccccc6c6ccccc56)cc4)cc3)cc2)cc1 ; c1ccc(-c2ccc(-c3ccc(N(c4ccc(-c5cc(-c6ccccc6)cc(-c6ccccc6)c5)cc4)c4cccc(-c5cc6ccccc6c6ccccc56)c4)cc3)cc2)cc1]

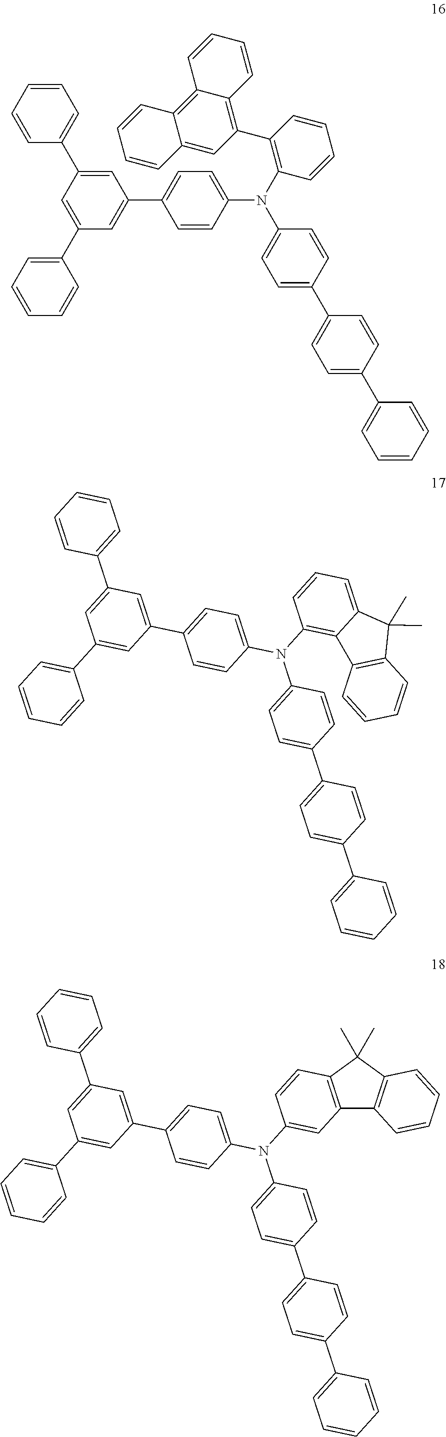 [SMILES: c1ccc(-c2ccc(-c3ccc(N(c4ccc(-c5cc(-c6ccccc6)cc(-c6ccccc6)c5)cc4)c4ccccc4-c4cc5ccccc5c5ccccc45)cc3)cc2)cc1 ; CC1(C)c2ccccc2-c2c(N(c3ccc(-c4ccc(-c5ccccc5)cc4)cc3)c3ccc(-c4cc(-c5ccccc5)cc(-c5ccccc5)c4)cc3)cccc21 ; CC1(C)c2ccccc2-c2cc(N(c3ccc(-c4ccc(-c5ccccc5)cc4)cc3)c3ccc(-c4cc(-c5ccccc5)cc(-c5ccccc5)c4)cc3)ccc21]

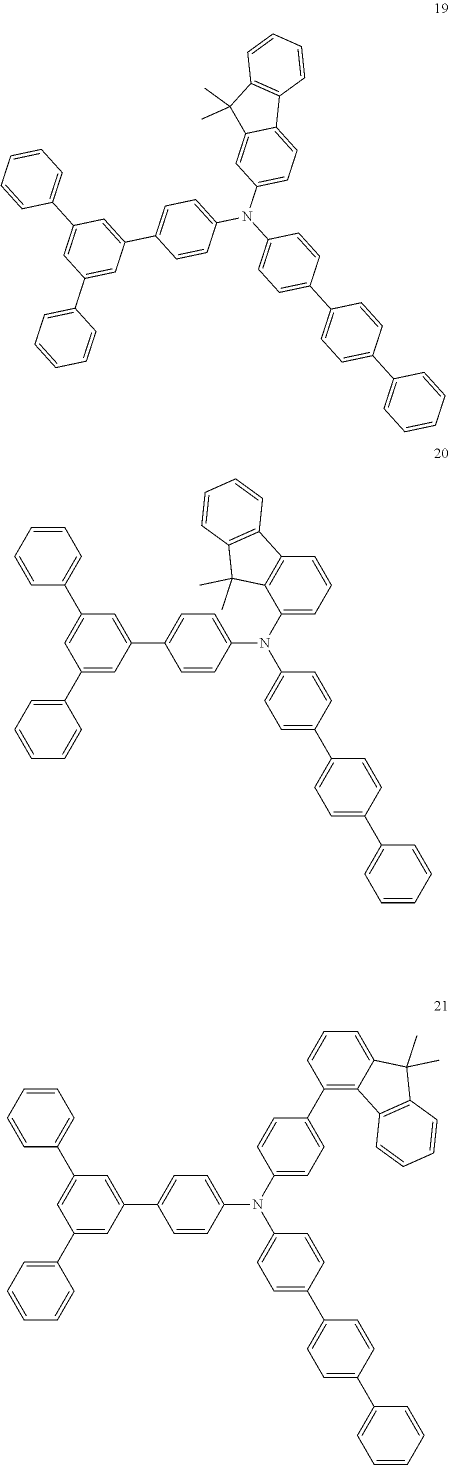 [SMILES: CC1(C)c2ccccc2-c2ccc(N(c3ccc(-c4ccc(-c5ccccc5)cc4)cc3)c3ccc(-c4cc(-c5ccccc5)cc(-c5ccccc5)c4)cc3)cc21 ; CC1(C)c2ccccc2-c2cccc(N(c3ccc(-c4ccc(-c5ccccc5)cc4)cc3)c3ccc(-c4cc(-c5ccccc5)cc(-c5ccccc5)c4)cc3)c21 ; CC1(C)c2ccccc2-c2c(-c3ccc(N(c4ccc(-c5ccc(-c6ccccc6)cc5)cc4)c4ccc(-c5cc(-c6ccccc6)cc(-c6ccccc6)c5)cc4)cc3)cccc21]

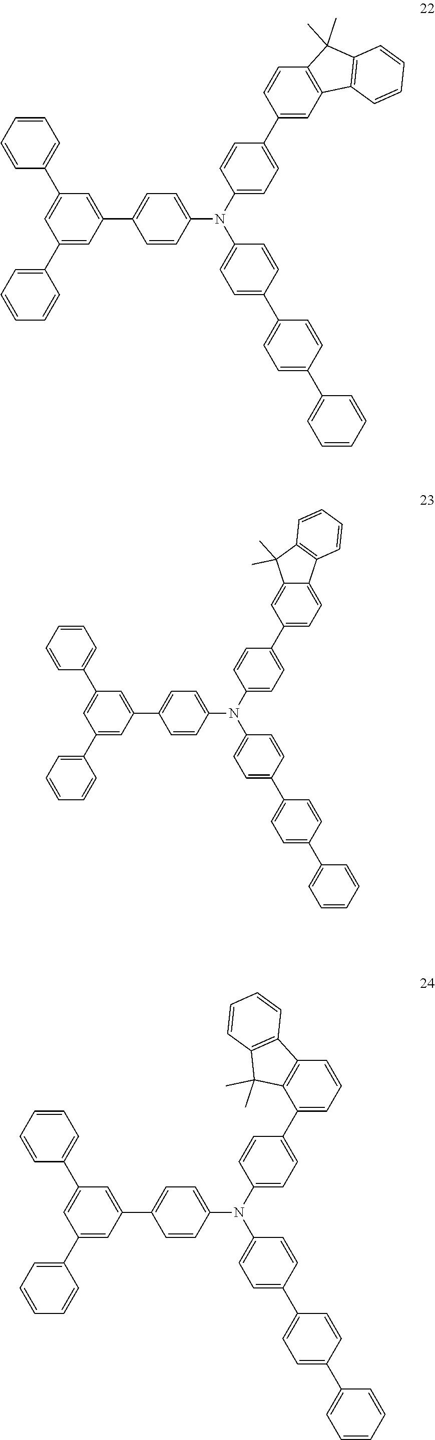 [SMILES: CC1(C)c2ccccc2-c2cc(-c3ccc(N(c4ccc(-c5ccc(-c6ccccc6)cc5)cc4)c4ccc(-c5cc(-c6ccccc6)cc(-c6ccccc6)c5)cc4)cc3)ccc21 ; CC1(C)c2ccccc2-c2ccc(-c3ccc(N(c4ccc(-c5ccc(-c6ccccc6)cc5)cc4)c4ccc(-c5cc(-c6ccccc6)cc(-c6ccccc6)c5)cc4)cc3)cc21 ; CC1(C)c2ccccc2-c2cccc(-c3ccc(N(c4ccc(-c5ccc(-c6ccccc6)cc5)cc4)c4ccc(-c5cc(-c6ccccc6)cc(-c6ccccc6)c5)cc4)cc3)c21]

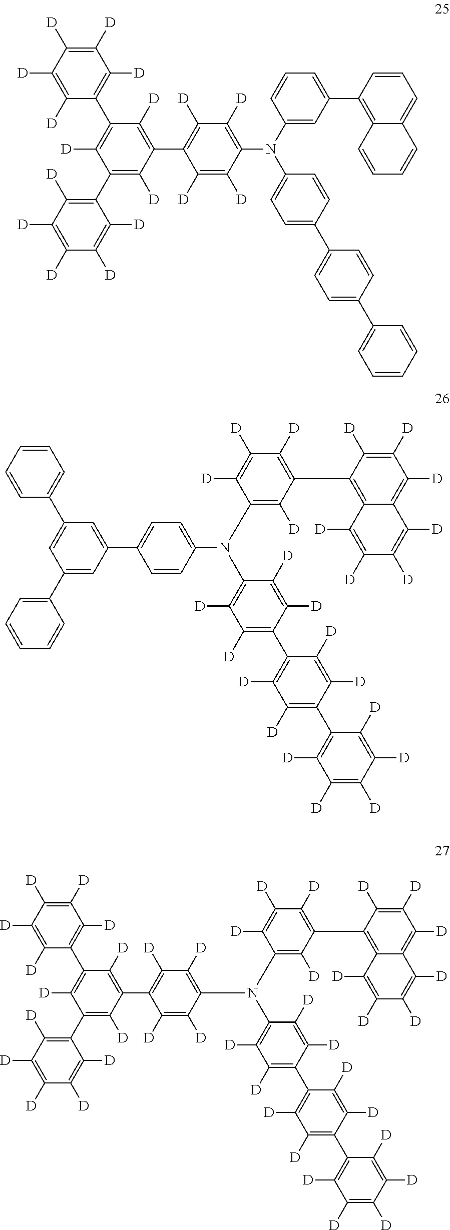

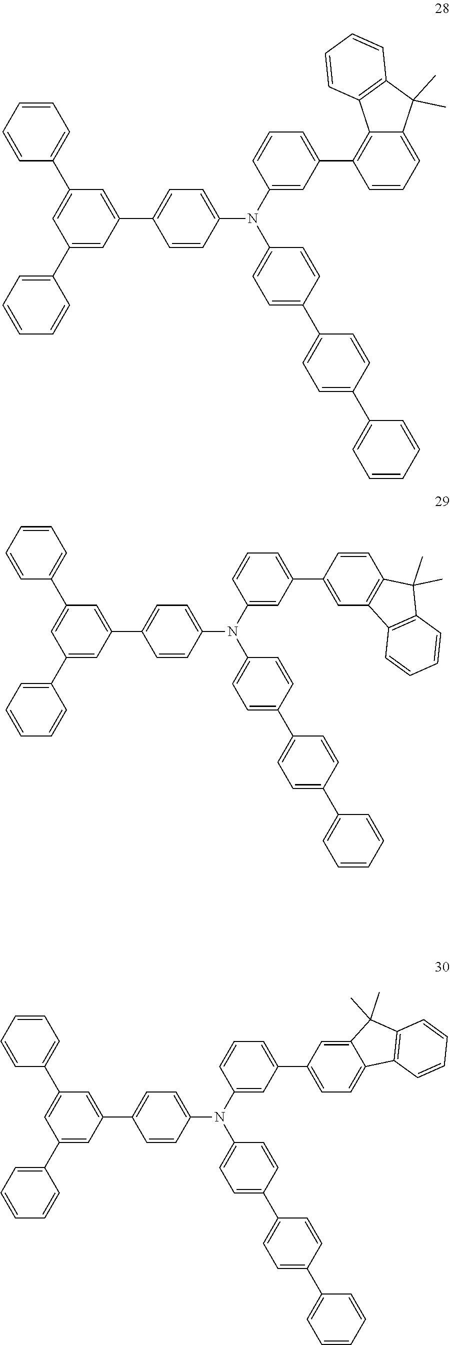 [SMILES: CC1(C)c2ccccc2-c2c(-c3cccc(N(c4ccc(-c5ccc(-c6ccccc6)cc5)cc4)c4ccc(-c5cc(-c6ccccc6)cc(-c6ccccc6)c5)cc4)c3)cccc21 ; CC1(C)c2ccccc2-c2cc(-c3cccc(N(c4ccc(-c5ccc(-c6ccccc6)cc5)cc4)c4ccc(-c5cc(-c6ccccc6)cc(-c6ccccc6)c5)cc4)c3)ccc21 ; CC1(C)c2ccccc2-c2ccc(-c3cccc(N(c4ccc(-c5ccc(-c6ccccc6)cc5)cc4)c4ccc(-c5cc(-c6ccccc6)cc(-c6ccccc6)c5)cc4)c3)cc21]

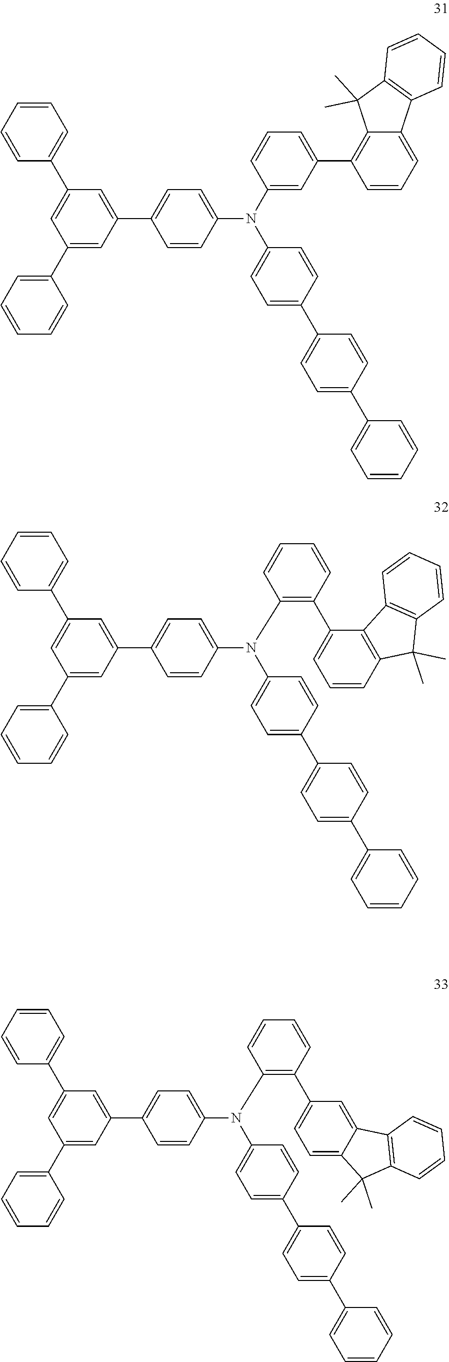 [SMILES: CC1(C)c2ccccc2-c2cccc(-c3cccc(N(c4ccc(-c5ccc(-c6ccccc6)cc5)cc4)c4ccc(-c5cc(-c6ccccc6)cc(-c6ccccc6)c5)cc4)c3)c21 ; CC1(C)c2ccccc2-c2c(-c3ccccc3N(c3ccc(-c4ccc(-c5ccccc5)cc4)cc3)c3ccc(-c4cc(-c5ccccc5)cc(-c5ccccc5)c4)cc3)cccc21 ; CC1(C)c2ccccc2-c2cc(-c3ccccc3N(c3ccc(-c4ccc(-c5ccccc5)cc4)cc3)c3ccc(-c4cc(-c5ccccc5)cc(-c5ccccc5)c4)cc3)ccc21]

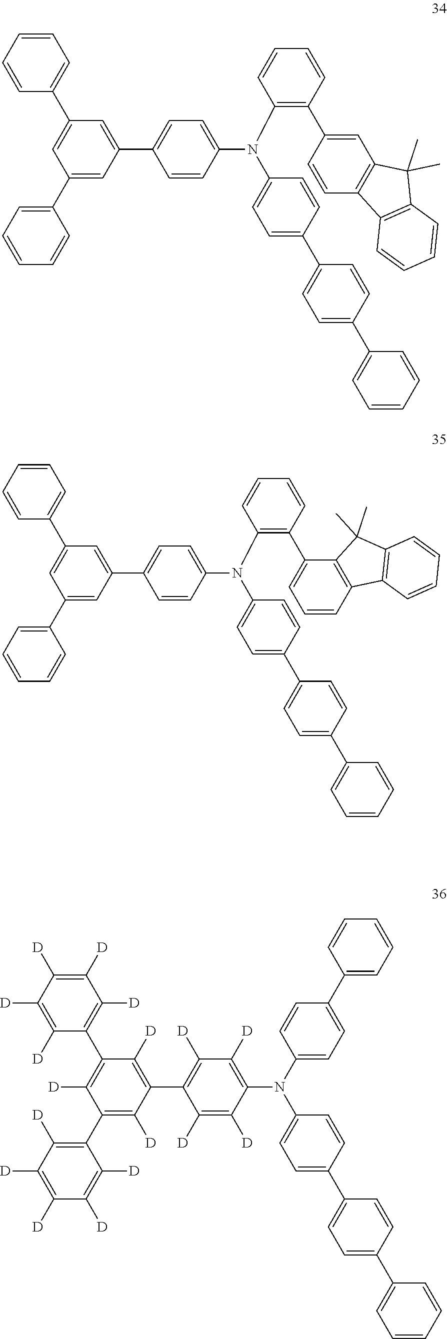 [SMILES: CC1(C)c2ccccc2-c2ccc(-c3ccccc3N(c3ccc(-c4ccc(-c5ccccc5)cc4)cc3)c3ccc(-c4cc(-c5ccccc5)cc(-c5ccccc5)c4)cc3)cc21 ; CC1(C)c2ccccc2-c2cccc(-c3ccccc3N(c3ccc(-c4ccc(-c5ccccc5)cc4)cc3)c3ccc(-c4cc(-c5ccccc5)cc(-c5ccccc5)c4)cc3)c21 ; [2H]c1c([2H])c([2H])c(-c2c([2H])c(-c3c([2H])c([2H])c([2H])c([2H])c3[2H])c([2H])c(-c3c([2H])c([2H])c(N(c4ccc(-c5ccccc5)cc4)c4ccc(-c5ccc(-c6ccccc6)cc5)cc4)c([2H])c3[2H])c2[2H])c([2H])c1[2H]]

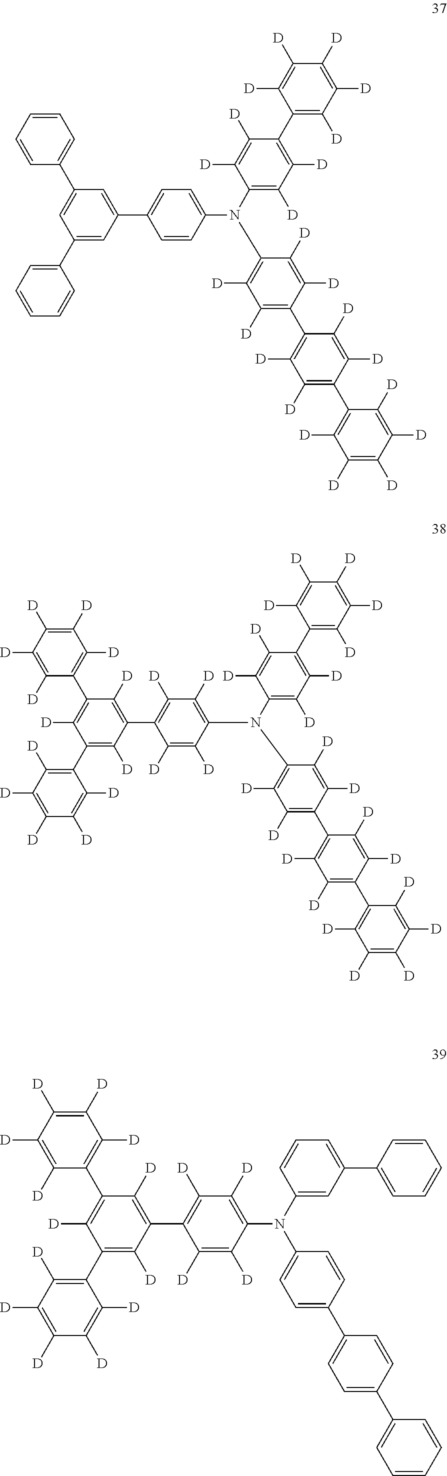

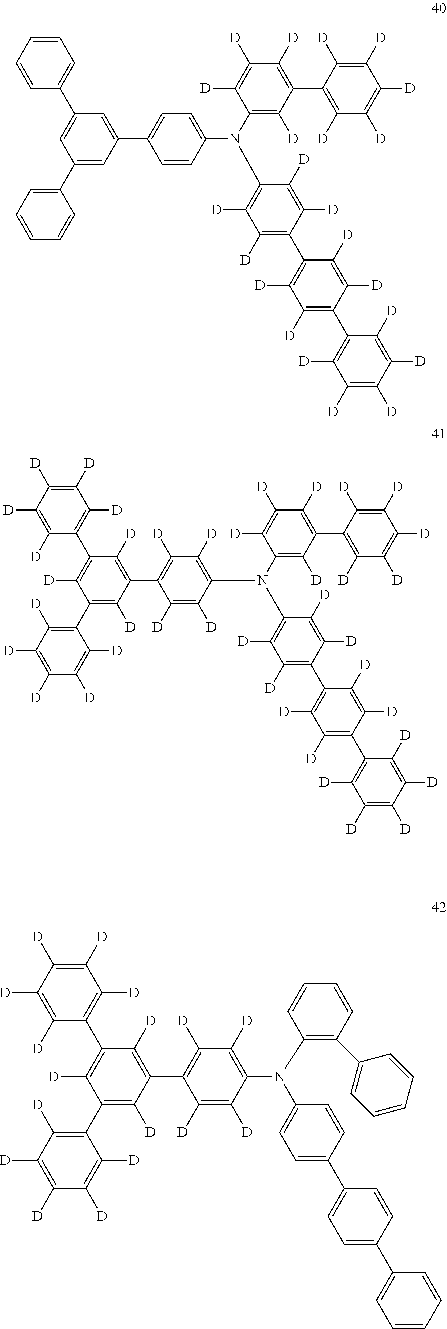

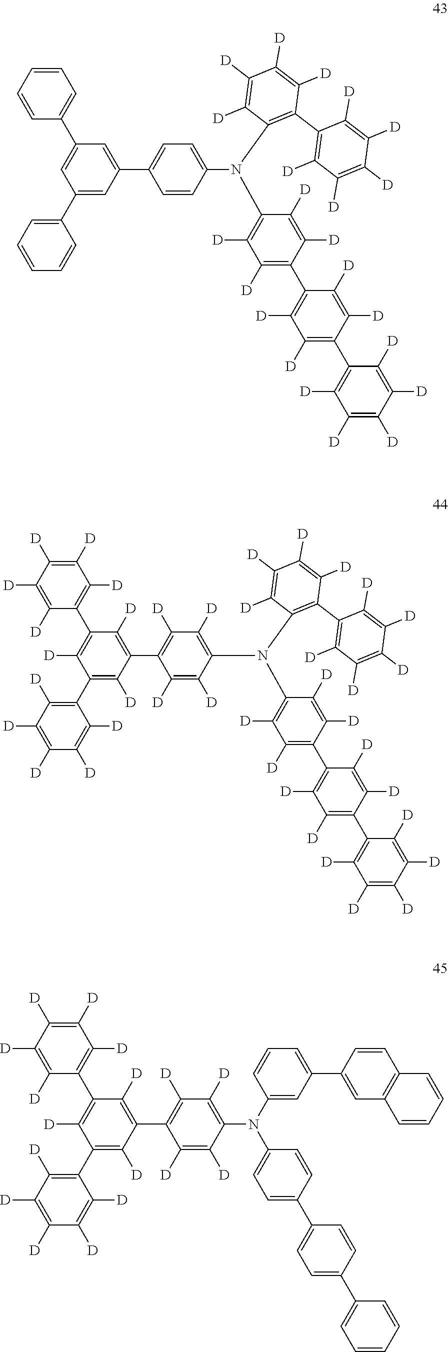

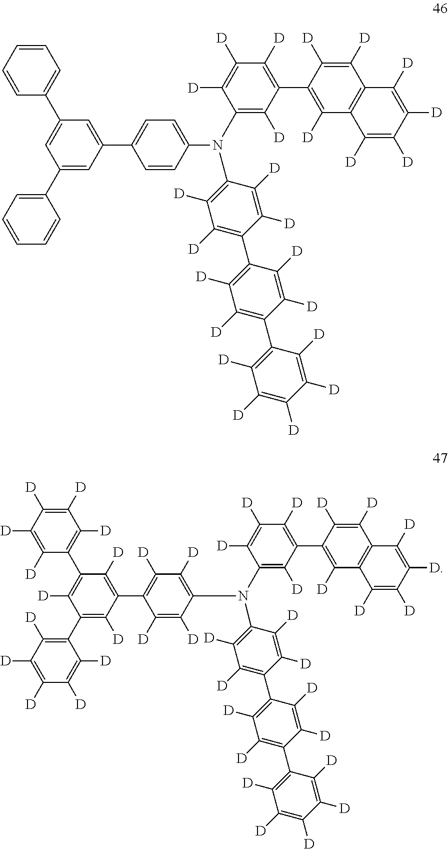

An organic light-emitting diode according to an embodiment of the present disclosure includes a first electrode (anode), a second electrode (cathode) facing the first electrode, and one or more organic layers disposed between the first electrode and second electrode, wherein at least one of the one or more organic layers is a hole transport layer or a hole transport auxiliary layer including the organic compound represented by Chemical Formula A.

The organic layers may include one or more of a hole injection layer (HIL), a hole transport layer (HTL), a hole transport auxiliary layer, an emitting layer (EML), an electron transport auxiliary layer, an electron transport layer (ETL), and an electron injection layer (EIL).

For example, the organic light-emitting diode may have a structure in which a first electrode, a hole injection layer (HIL), a hole transport layer (HTL), a hole transport auxiliary layer, an emitting layer (EML), an electron transport layer (ETL), an electron injection layer (EIL), and a second electrode are sequentially stacked.

Here, the organic layer including the organic compound represented by Chemical Formula A according to an embodiment of the present disclosure may be a hole transport layer (HTL) or a hole transport auxiliary layer.

The one or more organic layers may further include one or more selected from the group consisting of a hole injection layer, an emitting layer, an electron transport auxiliary layer, an electron transport layer, and an electron injection layer.

The first electrode may be an anode, and the first electrode may include a transparent and highly conductive material such as indium tin oxide (ITO), indium zinc oxide (IZO), tin oxide (SnO₂), or zinc oxide (ZnO).

The second electrode may be a cathode, and the second electrode may include a material such as lithium (Li), aluminum (Al), aluminum-lithium (Al—Li), calcium (Ca), magnesium (Mg), magnesium-indium (Mg—In), or magnesium-silver (Mg—Ag). In addition, in the case of a top-emitting organic light-emitting diode, a transparent second electrode through which light can be transmitted may be formed using indium tin oxide (ITO) or indium zinc oxide (IZO).

A capping layer (CPL) may be formed on a surface of the second electrode using a capping layer-forming composition.

In addition, an encapsulation layer (or protecting layer) for protecting the organic light-emitting diode from moisture, oxygen, and the like may be additionally disposed on the capping layer. Such an encapsulation layer (or protecting layer) may be formed from a curable adhesive composition containing an inorganic moisture absorbent.

An organic light-emitting diode according to an embodiment of the present disclosure includes a first electrode (anode), a second electrode (cathode) facing the first electrode, and one or more organic layers disposed between the first electrode and the second electrode, wherein at least one of the one or more organic layers is a hole transport auxiliary layer including an organic compound represented by Chemical Formula A.

Herein, when the organic layer including the organic compound represented by Chemical Formula A according to an embodiment of the present disclosure is a hole transport auxiliary layer, the hole injection layer, the emitting layer, the electron transport auxiliary layer, the electron transport layer, and the electron injection layer constituting the one or more organic layers may be defined as the following compounds, but are not limited thereto.

The hole injection layer or hole transport layer compound is not particularly limited, and any compound commonly used as a hole injection layer or hole transport layer compound may be employed. Non-limiting examples of the hole injection layer or hole transport layer compound may include phthalocyanine derivatives, porphyrin derivatives, triarylamine derivatives, indolocarbazole derivatives, and the like. Examples thereof include 1,4,5,8,9,11-hexaazatriphenylene-hexacarbonitrile (HAT-CN), copper phthalocyanine (CuPc), 4,4′,4″-tris(3-methylphenylamino)triphenylamine (m-MTDATA), 4,4′,4″-tris(3-methylphenylamino)phenoxybenzene (m-MTDAPB), 4,4′,4″-tri(N-carbazolyl)triphenylamine (TCTA), 4,4′,4″-tris(N-(2-naphthyl)-N-phenylamino)-triphenylamine (2-TNATA), N4,N4,N4′,N4′-tetra([1,1′-biphenyl]-4-yl)-[1,1′-biphenyl]-4,4′-diamine, bis(N-(1-naphthyl-n-phenyl))benzidine (α-NPD), N,N′-di(naphthalen-1-yl)-N,N′-biphenyl-benzidine (NPB), N,N′-biphenyl-N,N′-bis(3-methylphenyl)-1,1′-biphenyl-4,4′-diamine (TPD), and the like.

The compound included in the emitting layer is not particularly limited, and any compound commonly used as an emitting layer compound may be used. A single light-emitting compound or a light-emitting host compound may be used.

According to an embodiment of the present disclosure, the emitting layer may be a blue or green light-emitting layer. When the light-emitting layer is used as a blue light-emitting layer, excellent device performance may be achieved.

According to an embodiment of the present disclosure, when the light-emitting layer is used as a blue light-emitting layer, the CIEx and CIEy color coordinates are not particularly limited as long as they correspond to a blue light-emitting layer, but for example, the CIEx may be in a range of 0.10 to 0.15, and the CIEy may be in a range of 0.03 to 0.10.

The light-emitting compound of the emitting layer may include, but is not limited to, compounds capable of emitting light through phosphorescence, fluorescence, thermally activated delayed fluorescence (TADF, also referred to as E-type delayed fluorescence), triplet-triplet annihilation, or a combination of these processes. The light-emitting compound may be selected from various materials depending on a desired emission color. Non-limiting examples of the light-emitting compound include condensed ring derivatives such as phenanthrene, anthracene, pyrene, tetracene, pentacene, perylene, naphthopyrene, dibenzopyrene, rubrene, and chrysene, benzoxazole derivatives, benzothiazole derivatives, benzimidazole derivatives, benzotriazole derivatives, oxazole derivatives, oxadiazole derivatives, thiazole derivatives, imidazole derivatives, thiadiazole derivatives, triazole derivatives, pyrazoline derivatives, stilbene derivatives, thiophene derivatives, tetraphenylbutadiene derivatives, cyclopentadiene derivatives, bis(styryl) derivatives, bis(styryl)arylene derivatives, diazaindacene derivatives, furan derivatives, benzofuran derivatives, isobenzofuran derivatives, dibenzofuran derivatives, coumarin derivatives, dicyanomethylenepyran derivatives, dicyanomethylenethiopyran derivatives, polymethine derivatives, cyanine derivatives, oxobenzoanthracene derivatives, xanthene derivatives, rhodamine derivatives, fluorescein derivatives, pyrylium derivatives, carbostyril derivatives, acridine derivatives, oxazine derivatives, phenylene oxide derivatives, quinacridone derivatives, quinazoline derivatives, pyrrolopyridine derivatives, furopyridine derivatives, 1,2,5-thiadiazolopyrene derivatives, pyromethene derivatives, perinone derivatives, pyrrolopyrrole derivatives, squarylium derivatives, violanthrone derivatives, phenazine derivatives, acridone derivatives, deazaflavin derivatives, fluorene derivatives, benzofluorene derivatives, aromatic boron derivatives, aromatic nitrogen boron derivatives, and metal complexes (such as complexes of metals such as Ir, Pt, Au, Eu, Ru, Re, Ag, and Cu with heteroaromatic ring ligands) and the like. Examples thereof include N1,N1,N6,N6-tetrakis(4-(1-silyl)phenyl)pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB), platinumoctaethylporphyrin (PtOEP), Ir(ppy)₃, Ir(ppy)₂(acac), Ir(mppy)₃, Ir(PPy)₂(m-bppy), BtpIr(acac), Ir(btp)₂(acac), Ir(2-phq)₃, Hex-Ir(phq)₃, Ir(fbi)₂(acac), fac-tris(2-(3-p-xylyl)phenyl)pyridine iridium (III), Eu(dbm)₃(Phen), Ir(piq)₃, Ir(piq)₂(acac), Ir(Fliq)₂(acac), Ir(Flq)₂(acac), Ru(dtb-bpy)₃·2(PF₆), Ir(BT)₂(acac), Ir(DMP)₃, Ir(Mphq)₃, Ir(phq)₂tpy, fac-Ir(ppy)₂Pc, Ir(dp)PQ₂, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, and the like.

As the host compound of the light-emitting layer, a light-emitting host, a hole-transporting host, an electron-transporting host, or a combination thereof may be used. Non-limiting examples of light-emitting host compounds include fused ring derivatives such as anthracene or pyrene, bis(styryl) derivatives such as bis(styryl)anthracene derivatives or di(styryl)benzene derivatives, tetraphenylbutadiene derivatives, cyclopentadiene derivatives, fluorene derivatives, benzofluorene derivatives, n-phenylcarbazole (9-phenylcarbazole) derivatives, and carbazonitrile derivatives. Non-limiting examples of hole-transporting host materials include carbazole derivatives, dibenzofuran derivatives, dibenzothiophene derivatives, triarylamine derivatives, indolocarbazole derivatives, and benzoxazinophenoxazine derivatives. Non-limiting examples of electron-transporting host materials include pyridine derivatives, triazine derivatives, phosphine oxide derivatives, benzofuropyridine derivatives, and dibenzooxasiline derivatives. Examples thereof include 9,10-bis(2-naphthyl)anthracene (ADN), tris(8-hydroxyquinolinato)aluminum (Alq₃), BAlq (beryllium 8-hydroxyquinolinate), DPVBi (4,4′-bis(2,2-biphenylethenyl)-1,1′-biphenyl) series, spiro-DPVBi (spiro-4,4′-bis(2,2-biphenylethenyl)-1,1′-biphenyl), LiPBO (2-(2-benzoxazolyl)phenol lithium salt), bis(biphenylvinyl)benzene, aluminum-quinoline metal complexes, and metal complexes of imidazole, thiazole, and oxazole.

The electron injection layer or electron transport layer compound is not particularly limited, and any compound commonly used as an electron injection layer or electron transport layer compound may be used. Non-limiting examples of the electron injection layer or the electron transport layer compound include pyridine derivatives, naphthalene derivatives, anthracene derivatives, phenanthroline derivatives, perinone derivatives, coumarin derivatives, naphthalimide derivatives, anthraquinone derivatives, diphenoquinone derivatives, diphenylquinone derivatives, perylene derivatives, oxadiazole derivatives, thiophene derivatives, triazole derivatives, thiadiazole derivatives, metal complexes of oxine derivatives, quinolinol-based metal complexes, quinoxaline derivatives and polymers thereof, benzazole compounds, gallium complexes, pyrazole derivatives, perfluorinated phenylene derivatives, triazine derivatives, pyrazine derivatives, benzoquinoline derivatives, imidazopyridine derivatives, borane derivatives, benzimidazole derivatives, benzoxazole derivatives, benzothiazole derivatives, quinoline derivatives, oligopyridine derivatives such as terpyridine, bipyridine derivatives, terpyridine derivatives, naphthyridine derivatives, aldazine derivatives, carbazole derivatives, indole derivatives, phosphine oxide derivatives, bis(styryl) derivatives, quinolinol-based metal complexes, hydroxazole-based metal complexes, azomethine-based metal complexes, tropolone-based metal complexes, flavonol-based metal complexes, benzoquinoline-based metal complexes, and metal salts. These materials may be used alone or in combination with other materials. Examples thereof include 2-(4-(9,10-di(naphthalen-2-yl)anthracen-2-yl)phenyl)-1-phenyl-1H-benzo[d]imidazole, tris(8-hydroxyquinolinato)aluminum (Alq₃), LiF, Liq, Li₂O, BaO, NaCl, and CsF.

The electron transport auxiliary layer compound disposed between the electron transport layer and the emitting layer is not particularly limited, and any compound commonly used as an electron transport auxiliary layer compound may be used. For example, the electron transport auxiliary layer may include a pyrimidine derivative, and the like.

According to an embodiment of the present disclosure, the organic light-emitting diode may be of a top-emitting or a bottom-emitting diode.

According to an embodiment of the present disclosure, the organic light-emitting diode may be used in a display device.

According to an embodiment of the present disclosure, the organic light-emitting diode may be applied to, but is not limited to, transparent display devices, mobile display devices, flexible display devices, and the like.

An organic light-emitting diode according to an embodiment of the present disclosure include a tandem structure including a plurality of light-emitting stacks disposed between an anode and a cathode.

Hereinafter, representative examples of methods for synthesizing the compounds will be described. However, the methods for synthesizing the compounds of the present disclosure are not limited to the exemplary methods described below, and the practice of the present disclosure is not limited to the following Examples and Experimental Examples.

### Synthesis Example

A representative synthesis example for Compound 4 is described, and compounds 5 to 12, 21, 23, 27, 28, 30, 32, 34, 38, 41, 44, 47 and compounds of Chemical Formula A according to the present disclosure may be synthesized similarly to the reaction of Compound 4.

In the following reaction schemes, solvents, catalysts, protecting groups, leaving groups, reaction temperatures, reaction times, and the like are provided as representative examples, and any equivalent solvents, catalysts, protecting groups, leaving groups, reaction temperatures, reaction times, reactant equivalents, and the like, may also be used.

### 1. Synthesis of Compound 4

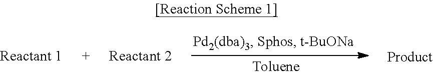

Under a nitrogen atmosphere, reactant 1 (22 mmol) and reactant 2 (20 mmol) for preparing Compound 4, Pd₂(dba)₃ (0.8 mmol), SPhos (1.6 mmol), t-BuONa (50 mmol), and toluene were added to a reaction flask. The mixture was stirred and refluxed. The organic layer was extracted using toluene and water. The extracted solution was treated with MgSO₄ to remove residual moisture, concentrated under reduced pressure, purified by column chromatography, and then recrystallized to obtain Compound 4, which is an organic compound represented by Chemical Formula A according to the present disclosure.

Representative products synthesized according to Reaction Scheme 1, along with Reactant 1 and Reactant 2 used for their synthesis, are shown in Table 1 below. Compounds corresponding to Chemical Formula A and similar compounds thereof may be synthesized through the Synthetic Examples of Reaction Scheme 1.

TABLE 1

| Syn- | | |
|---|---|---|
| thesis | | |
| Ex- | | |
| am- | | |
| ples | Reactant 1 | Reactant 2 |
| 1 | 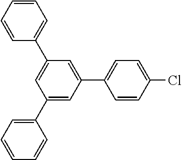 [SMILES: Clc1ccc(-c2cc(-c3ccccc3)cc(-c3ccccc3)c2)cc1] | 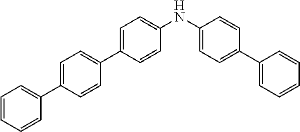 [SMILES: c1ccc(-c2ccc(Nc3ccc(-c4ccc(-c5ccccc5)cc4)cc3)cc2)cc1] |
| 2 | 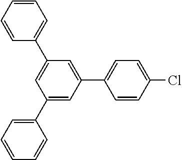 [SMILES: Clc1ccc(-c2cc(-c3ccccc3)cc(-c3ccccc3)c2)cc1] | 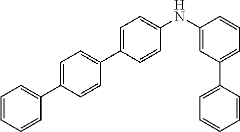 [SMILES: c1ccc(-c2ccc(-c3ccc(Nc4cccc(-c5ccccc5)c4)cc3)cc2)cc1] |
| 3 | 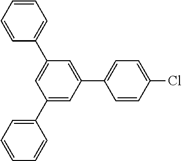 [SMILES: Clc1ccc(-c2cc(-c3ccccc3)cc(-c3ccccc3)c2)cc1] | 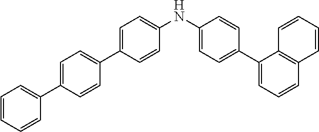 [SMILES: c1ccc(-c2ccc(-c3ccc(Nc4ccc(-c5cccc6ccccc56)cc4)cc3)cc2)cc1] |
| 4 | 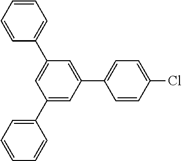 [SMILES: Clc1ccc(-c2cc(-c3ccccc3)cc(-c3ccccc3)c2)cc1] | 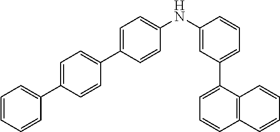 [SMILES: c1ccc(-c2ccc(-c3ccc(Nc4cccc(-c5cccc6ccccc56)c4)cc3)cc2)cc1] |
| 5 | 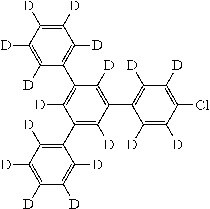 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c([2H])c3[2H])c2[2H])c([2H])c1[2H]] | 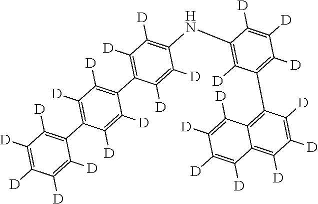 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] |
| 6 | 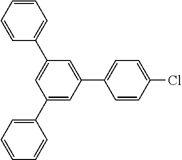 [SMILES: Clc1ccc(-c2cc(-c3ccccc3)cc(-c3ccccc3)c2)cc1] | 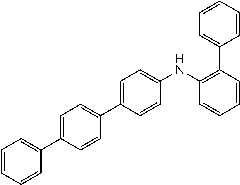 [SMILES: c1ccc(-c2ccc(-c3ccc(Nc4ccccc4-c4ccccc4)cc3)cc2)cc1] |
| 7 | 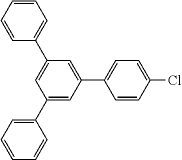 [SMILES: Clc1ccc(-c2cc(-c3ccccc3)cc(-c3ccccc3)c2)cc1] | 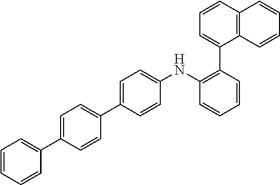 [SMILES: c1ccc(-c2ccc(-c3ccc(Nc4ccccc4-c4cccc5ccccc45)cc3)cc2)cc1] |
| 8 | 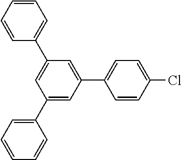 [SMILES: Clc1ccc(-c2cc(-c3ccccc3)cc(-c3ccccc3)c2)cc1] | 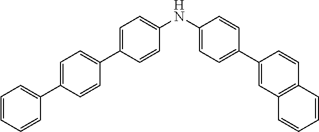 [SMILES: c1ccc(-c2ccc(-c3ccc(Nc4ccc(-c5ccc6ccccc6c5)cc4)cc3)cc2)cc1] |
| 9 | 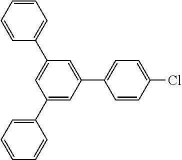 [SMILES: Clc1ccc(-c2cc(-c3ccccc3)cc(-c3ccccc3)c2)cc1] | 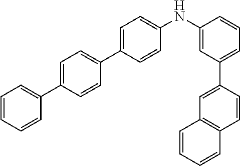 [SMILES: c1ccc(-c2ccc(-c3ccc(Nc4cccc(-c5ccc6ccccc6c5)c4)cc3)cc2)cc1] |
| 10 | 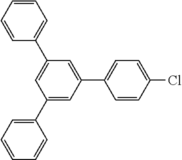 [SMILES: Clc1ccc(-c2cc(-c3ccccc3)cc(-c3ccccc3)c2)cc1] | 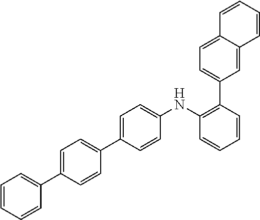 [SMILES: c1ccc(-c2ccc(-c3ccc(Nc4ccccc4-c4ccc5ccccc5c4)cc3)cc2)cc1] |
| 11 | 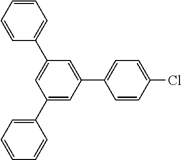 [SMILES: Clc1ccc(-c2cc(-c3ccccc3)cc(-c3ccccc3)c2)cc1] | 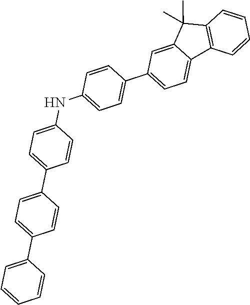 [SMILES: CC1(C)c2ccccc2-c2ccc(-c3ccc(Nc4ccc(-c5ccc(-c6ccccc6)cc5)cc4)cc3)cc21] |
| 12 | 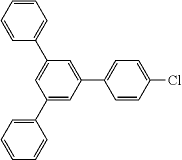 [SMILES: Clc1ccc(-c2cc(-c3ccccc3)cc(-c3ccccc3)c2)cc1] | 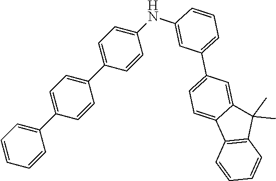 [SMILES: CC1(C)c2ccccc2-c2ccc(-c3cccc(Nc4ccc(-c5ccc(-c6ccccc6)cc5)cc4)c3)cc21] |
| 13 | 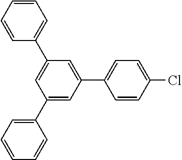 [SMILES: Clc1ccc(-c2cc(-c3ccccc3)cc(-c3ccccc3)c2)cc1] | 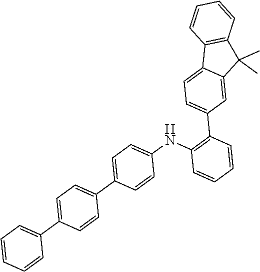 [SMILES: CC1(C)c2ccccc2-c2ccc(-c3ccccc3Nc3ccc(-c4ccc(-c5ccccc5)cc4)cc3)cc21] |
| 14 | 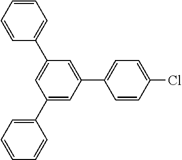 [SMILES: Clc1ccc(-c2cc(-c3ccccc3)cc(-c3ccccc3)c2)cc1] | 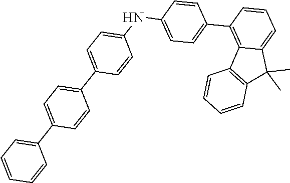 [SMILES: CC1(C)c2ccccc2-c2c(-c3ccc(Nc4ccc(-c5ccc(-c6ccccc6)cc5)cc4)cc3)cccc21] |
| 15 | 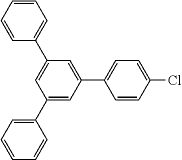 [SMILES: Clc1ccc(-c2cc(-c3ccccc3)cc(-c3ccccc3)c2)cc1] | 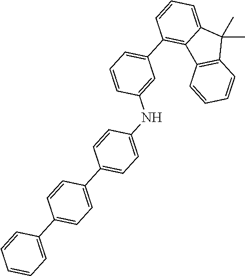 [SMILES: CC1(C)c2ccccc2-c2c(-c3cccc(Nc4ccc(-c5ccc(-c6ccccc6)cc5)cc4)c3)cccc21] |
| 16 | 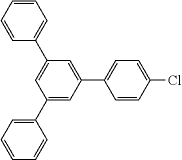 [SMILES: Clc1ccc(-c2cc(-c3ccccc3)cc(-c3ccccc3)c2)cc1] | 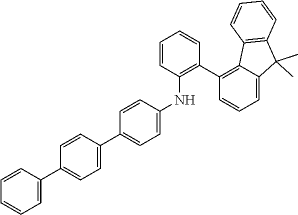 [SMILES: CC1(C)c2ccccc2-c2c(-c3ccccc3Nc3ccc(-c4ccc(-c5ccccc5)cc4)cc3)cccc21] |
| 17 | 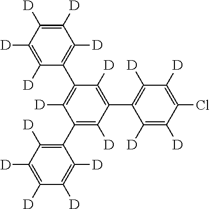 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c([2H])c3[2H])c2[2H])c([2H])c1[2H]] | 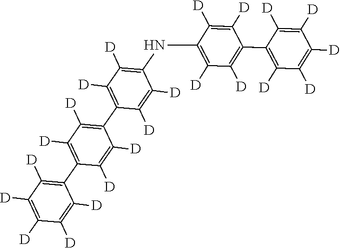 [SMILES: [2H]c1c([2H])c([2H])c(-c2c([2H])c([2H])c(Nc3c([2H])c([2H])c(-c4c([2H])c([2H])c(-c5c([2H])c([2H])c([2H])c([2H])c5[2H])c([2H])c4[2H])c([2H])c3[2H])c([2H])c2[2H])c([2H])c1[2H]] |
| 18 | 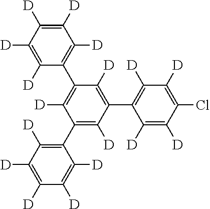 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c([2H])c3[2H])c2[2H])c([2H])c1[2H]] | 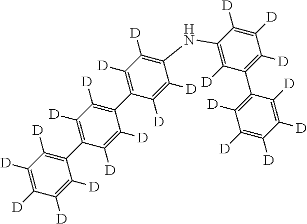 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c([2H])c5[2H])c4[2H])c([2H])c3[2H])c([2H])c2[2H])c([2H])c1[2H]] |
| 19 | 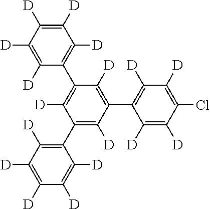 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c([2H])c3[2H])c2[2H])c([2H])c1[2H]] | 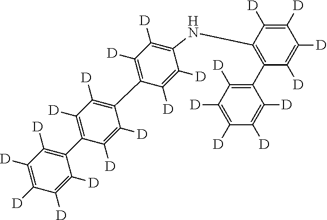 [SMILES: [2H]c1c([2H])c([2H])c(-c2c([2H])c([2H])c(-c3c([2H])c([2H])c(Nc4c([2H])c([2H])c([2H])c([2H])c4-c4c([2H])c([2H])c([2H])c([2H])c4[2H])c([2H])c3[2H])c([2H])c2[2H])c([2H])c1[2H]] |
| 20 | 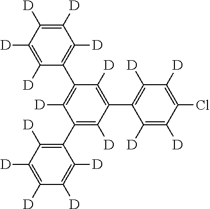 [SMILES: [2H]c1c([2H])c([2H])c(-c2c([2H])c(-c3c([2H])c([2H])c([2H])c([2H])c3[2H])c([2H])c(-c3c([2H])c([2H])c(Cl)c([2H])c3[2H])c2[2H])c([2H])c1[2H]] | 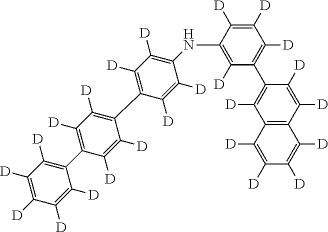 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] |

| Syn- | | | |
|---|---|---|---|
| thesis | | Amount | |
| Ex- | | of | |
| am- | | Product | |
| ples | Product | (Yield) | [M + H]⁺ |
| 1 | 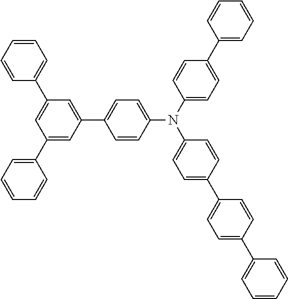 [SMILES: c1ccc(-c2ccc(-c3ccc(N(c4ccc(-c5ccccc5)cc4)c4ccc(-c5cc(-c6ccccc6)cc(-c6ccccc6)c5)cc4)cc3)cc2)cc1] | 12.9 g (78%) | 701.31 |
| 2 | 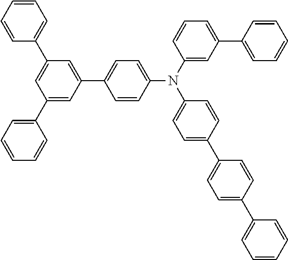 [SMILES: c1ccc(-c2ccc(-c3ccc(N(c4ccc(-c5cc(-c6ccccc6)cc(-c6ccccc6)c5)cc4)c4cccc(-c5ccccc5)c4)cc3)cc2)cc1] | 11.7 g (71%) | 701.31 |
| 3 | 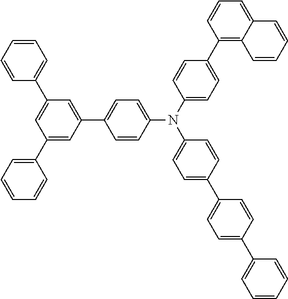 [SMILES: c1ccc(-c2ccc(-c3ccc(N(c4ccc(-c5cc(-c6ccccc6)cc(-c6ccccc6)c5)cc4)c4ccc(-c5cccc6ccccc56)cc4)cc3)cc2)cc1] | 11.7 g (71%) | 751.32 |
| 4 | 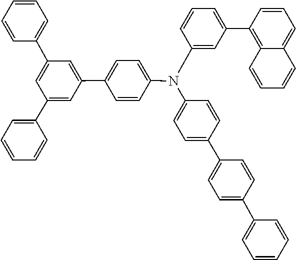 [SMILES: c1ccc(-c2ccc(-c3ccc(N(c4ccc(-c5cc(-c6ccccc6)cc(-c6ccccc6)c5)cc4)c4cccc(-c5cccc6ccccc56)c4)cc3)cc2)cc1] | 11.4 g (69%) | 751.32 |
| 5 | 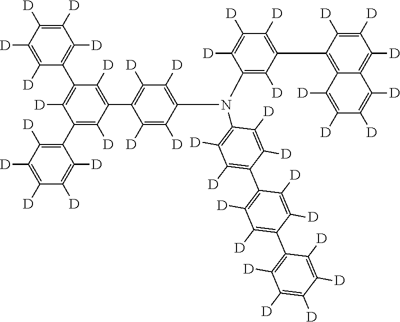 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | 11.6 g (67%) | 789.56 |
| 6 | 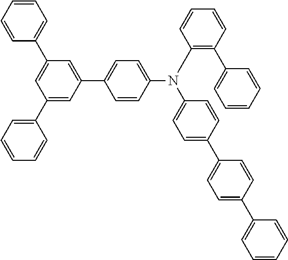 [SMILES: c1ccc(-c2ccc(-c3ccc(N(c4ccc(-c5cc(-c6ccccc6)cc(-c6ccccc6)c5)cc4)c4ccccc4-c4ccccc4)cc3)cc2)cc1] | 9.54 g (68%) | 701.31 |
| 7 | 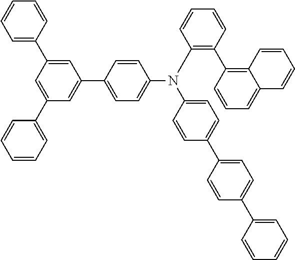 [SMILES: c1ccc(-c2ccc(-c3ccc(N(c4ccc(-c5cc(-c6ccccc6)cc(-c6ccccc6)c5)cc4)c4ccccc4-c4cccc5ccccc45)cc3)cc2)cc1] | 9.32 g (62%) | 751.32 |
| 8 | 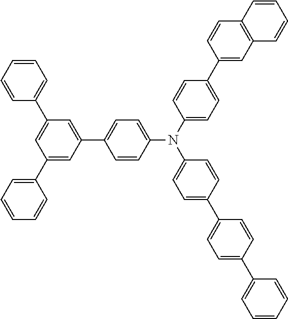 [SMILES: c1ccc(-c2ccc(-c3ccc(N(c4ccc(-c5cc(-c6ccccc6)cc(-c6ccccc6)c5)cc4)c4ccc(-c5ccc6ccccc6c5)cc4)cc3)cc2)cc1] | 10.83 g (72%) | 751.32 |
| 9 | 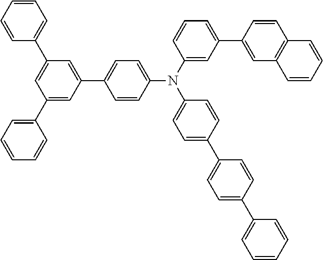 [SMILES: c1ccc(-c2ccc(-c3ccc(N(c4ccc(-c5cc(-c6ccccc6)cc(-c6ccccc6)c5)cc4)c4cccc(-c5ccc6ccccc6c5)c4)cc3)cc2)cc1] | 10.53 g (70%) | 751.32 |
| 10 | 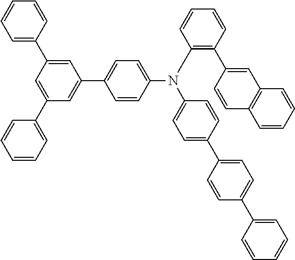 [SMILES: c1ccc(-c2ccc(-c3ccc(N(c4ccc(-c5cc(-c6ccccc6)cc(-c6ccccc6)c5)cc4)c4ccccc4-c4ccc5ccccc5c4)cc3)cc2)cc1] | 9.92 g (66%) | 751.32 |
| 11 | 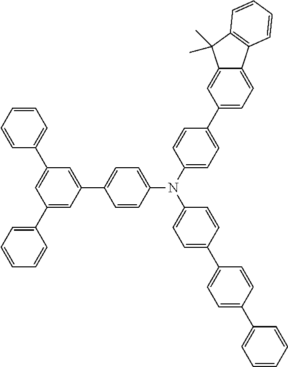 [SMILES: CC1(C)c2ccccc2-c2ccc(-c3ccc(N(c4ccc(-c5ccc(-c6ccccc6)cc5)cc4)c4ccc(-c5cc(-c6ccccc6)cc(-c6ccccc6)c5)cc4)cc3)cc21] | 12.27 g (75%) | 817.37 |
| 12 | 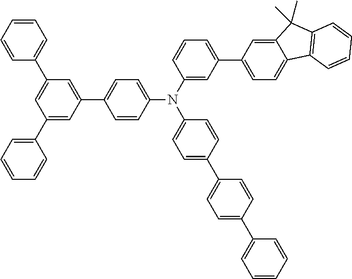 [SMILES: CC1(C)c2ccccc2-c2ccc(-c3cccc(N(c4ccc(-c5ccc(-c6ccccc6)cc5)cc4)c4ccc(-c5cc(-c6ccccc6)cc(-c6ccccc6)c5)cc4)c3)cc21] | 12.43 g (76%) | 817.37 |
| 13 | 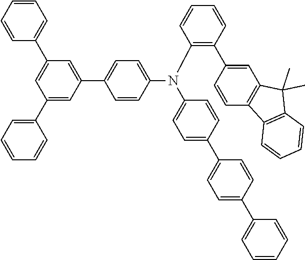 [SMILES: CC1(C)c2ccccc2-c2ccc(-c3ccccc3N(c3ccc(-c4ccc(-c5ccccc5)cc4)cc3)c3ccc(-c4cc(-c5ccccc5)cc(-c5ccccc5)c4)cc3)cc21] | 10.96 g (67%) | 817.37 |
| 14 | 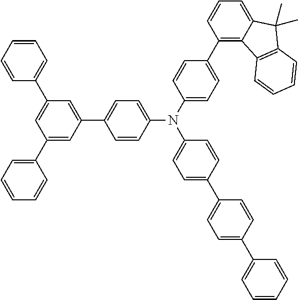 [SMILES: CC1(C)c2ccccc2-c2c(-c3ccc(N(c4ccc(-c5ccc(-c6ccccc6)cc5)cc4)c4ccc(-c5cc(-c6ccccc6)cc(-c6ccccc6)c5)cc4)cc3)cccc21] | 11.29 g (69%) | 817.37 |
| 15 | 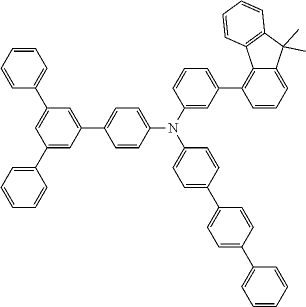 [SMILES: CC1(C)c2ccccc2-c2c(-c3cccc(N(c4ccc(-c5ccc(-c6ccccc6)cc5)cc4)c4ccc(-c5cc(-c6ccccc6)cc(-c6ccccc6)c5)cc4)c3)cccc21] | 11.62 g (71%) | 817.37 |
| 16 | 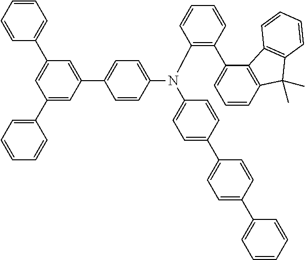 [SMILES: CC1(C)c2ccccc2-c2c(-c3ccccc3N(c3ccc(-c4ccc(-c5ccccc5)cc4)cc3)c3ccc(-c4cc(-c5ccccc5)cc(-c5ccccc5)c4)cc3)cccc21] | 9.98 g (61%) | 817.37 |
| 17 | 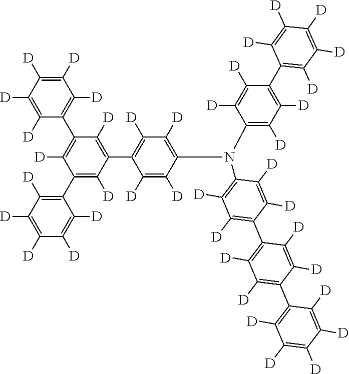 [SMILES: [2H]c1c([2H])c([2H])c(-c2c([2H])c([2H])c(-c3c([2H])c([2H])c(N(c4c([2H])c([2H])c(-c5c([2H])c([2H])c([2H])c([2H])c5[2H])c([2H])c4[2H])c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c([2H])c3[2H])c([2H])c2[2H])c([2H])c1[2H]] | 10.97 g (74%) | 740.55 |
| 18 | 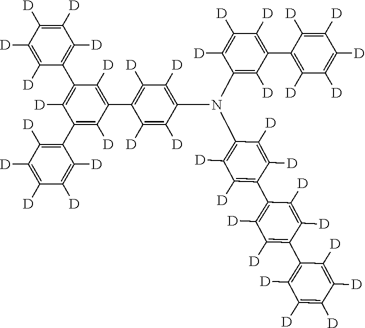 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c([2H])c5[2H])c4[2H])c([2H])c3[2H])c([2H])c2[2H])c([2H])c1[2H]] | 10.82 g (73%) | 740.55 |
| 19 | 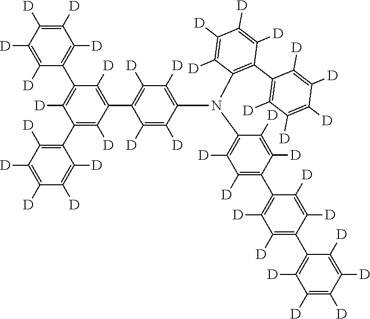 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c([2H])c4-c4c([2H])c([2H])c([2H])c([2H])c4[2H])c([2H])c3[2H])c([2H])c2[2H])c([2H])c1[2H]] | 10.37 g (70%) | 740.55 |
| 20 | 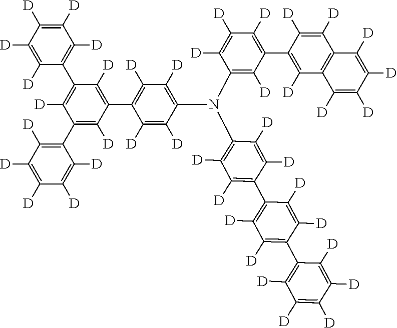 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | 11.89 g (75%) | 792.58 |

### [Experimental Example 1] Performance Evaluation of Organic Light-Emitting Diodes (Blue Organic Light Emitting Diode)

For the organic light-emitting diodes of Examples 1 to 20 and Comparative Examples 1 to 6 manufactured as described below, driving voltage (V), efficiency (Cd/A), and Y color coordinate (CIE 1931 standard, CIEy) were measured by applying a current of 10 mA/cm² using a KONICA MINOLTA CS-2000. The Blue Index (BI=Cd/A(B)/CIEy(B)) was calculated and recorded using the efficiency and color coordinate values. In addition, the lifetime (LT95, hrs) was measured using a McScience M6000 under constant-current driving at 10 mA/cm², with the lifetime defined as the time required for the luminance to decrease to 95% of the initial luminance. The measurement results are shown in Table 2 below.

### Example 1—Manufacturing of Organic Light-Emitting Diode (Blue Emitting Layer)

A substrate on which indium tin oxide (ITO, 100 nm), serving as a first electrode (anode) of an organic light-emitting diode, was deposited was patterned by a photolithography process to define regions of a second electrode (cathode), the first electrode (anode), and an insulating layer. Thereafter, the surface of the first electrode (ITO) was treated by UV-ozone treatment and O₂:N₂ plasma for the purpose of increasing the work function and cleaning the surface.

Next, on the anode, a hole injection layer (HIL) was formed to a thickness of 10 nm by depositing a mixture of NDP-9 (2-(7-dicyanomethylene-1,3,4,5,6,8,9,10-octafluoro-7H-pyren-2-ylidene)-malononitrile) and N4,N4,N4′,N4′-tetra([1,1′-biphenyl]-4-yl)-[1,1′-biphenyl]-4,4′-diamine) at a ratio of 3:97. Then, on the hole injection layer, a hole transport layer (HTL) was formed to a thickness of 100 nm by vacuum-depositing N4,N4,N4′,N4′-tetra([1,1′-biphenyl]-4-yl)-[1,1′-biphenyl]-4,4′-diamine. On the HTL, a hole transport auxiliary layer was formed to a thickness of 15 nm using Compound 4.

On the hole transport auxiliary layer, a blue emitting layer was deposited to a thickness of 25 nm using 9,10-bis(2-naphthyl)anthracene (ADN) as the host and 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB) as the dopant, wherein the mixing ratio (by weight) of host:dopant was 97:3. On the blue emitting layer, an electron transport layer (ETL) was deposited to a thickness of 25 nm by co-depositing 2-(4-(9,10-di(naphthalene-2-yl)anthracene-2-yl)phenyl)-1-phenyl-1H-benzo[d]imidazole and Liq at a weight ratio of 1:1. On the electron transport layer (ETL), an electron injection layer (Liq) was deposited to a thickness of 1 nm, and a cathode was deposited to a thickness of 16 nm by co-depositing magnesium and silver in a weight ratio of 1:4. On the cathode, a capping layer was deposited to a thickness of 60 nm using N4,N4′-bis(4-(benzo[d]oxazol-2-yl)phenyl)-N4,N4′-diphenyl-[1,1′-biphenyl]-4,4′-diamine (DNTPD). A seal cap containing a moisture absorbent was bonded onto the capping layer using a UV-curable adhesive to form an encapsulation layer (or a protecting layer) capable of protecting the organic light-emitting diode from oxygen or moisture in the atmosphere, thereby manufacturing the organic light-emitting diode.

### Examples 2 to 20

Organic light-emitting diodes of Examples 2 to 20 were each manufactured in the same manner as in Example 1, except that Compound 4 used as the hole transport auxiliary layer material in Example 1 was replaced with the compounds listed in Table 2 below.

### Comparative Examples 1 to 6

Organic light-emitting diodes of Comparative Examples 1 to 6 were each manufactured in the same manner as in Example 1, except that Compound 4 used as the hole transport auxiliary layer material in Example 1 was replaced with Comparative Compounds 1 to 6 shown in Table 2 below.

TABLE 2

| | | Driv- | | | | |
|---|---|---|---|---|---|---|
| | | ing | Effi- | | | Life- |
| Examples/ | | Volt- | ciency | | | time |
| Comp. | Hole Transport Auxiliary | age | (Cd/ | | | (LT95) |
| Examples | Layer Compound | (V) | A) | CIEy | BI | (hrs) |
| Example 1 | 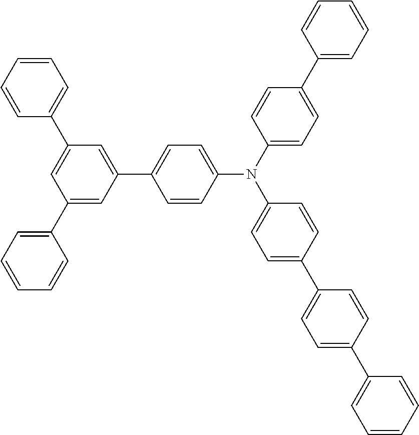 [SMILES: c1ccc(-c2ccc(-c3ccc(N(c4ccc(-c5ccccc5)cc4)c4ccc(-c5cc(-c6ccccc6)cc(-c6ccccc6)c5)cc4)cc3)cc2)cc1] | 3.35 | 8.2 | 0.045 | 182.2 | 438 |
| | Compound 4 | | | | | |
| Example 2 | 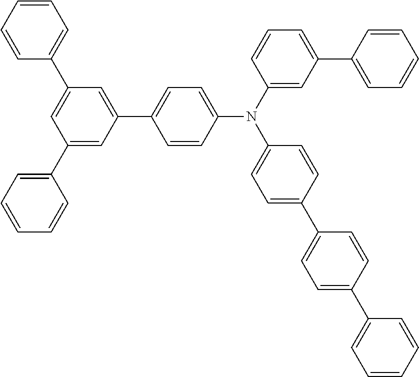 [SMILES: c1ccc(-c2ccc(-c3ccc(N(c4ccc(-c5cc(-c6ccccc6)cc(-c6ccccc6)c5)cc4)c4cccc(-c5ccccc5)c4)cc3)cc2)cc1] | 3.30 | 8.5 | 0.045 | 188.9 | 455 |
| | Compound 5 | | | | | |
| Example 3 | 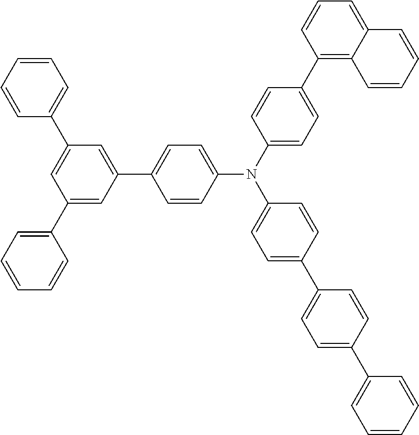 [SMILES: c1ccc(-c2ccc(-c3ccc(N(c4ccc(-c5cc(-c6ccccc6)cc(-c6ccccc6)c5)cc4)c4ccc(-c5cccc6ccccc56)cc4)cc3)cc2)cc1] | 3.40 | 7.6 | 0.045 | 168.9 | 415 |
| | Compound 7 | | | | | |
| Example 4 | 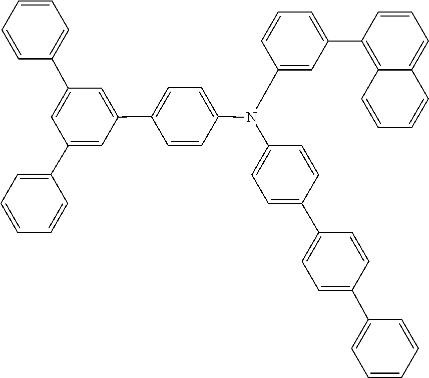 [SMILES: c1ccc(-c2ccc(-c3ccc(N(c4ccc(-c5cc(-c6ccccc6)cc(-c6ccccc6)c5)cc4)c4cccc(-c5cccc6ccccc56)c4)cc3)cc2)cc1] | 3.36 | 7.9 | 0.045 | 175.6 | 428 |
| | Compound 9 | | | | | |
| Example 5 | 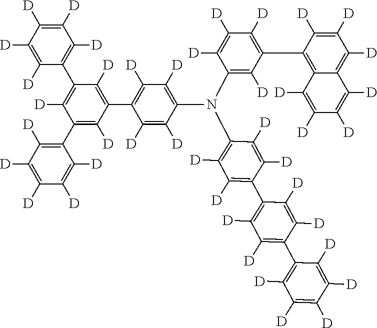 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | 3.43 | 7.9 | 0.045 | 175.6 | 469 |
| | Compound 27 | | | | | |
| Example 6 | 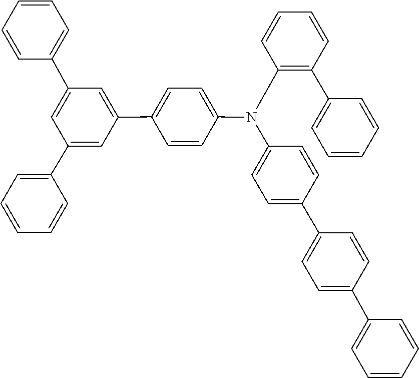 [SMILES: c1ccc(-c2ccc(-c3ccc(N(c4ccc(-c5cc(-c6ccccc6)cc(-c6ccccc6)c5)cc4)c4ccccc4-c4ccccc4)cc3)cc2)cc1] | 3.33 | 8.4 | 0.045 | 186.7 | 443 |
| | Compound 6 | | | | | |
| Example 7 | 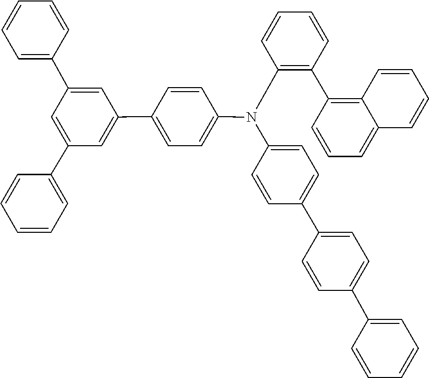 [SMILES: c1ccc(-c2ccc(-c3ccc(N(c4ccc(-c5cc(-c6ccccc6)cc(-c6ccccc6)c5)cc4)c4ccccc4-c4cccc5ccccc45)cc3)cc2)cc1] | 3.39 | 7.8 | 0.045 | 173.3 | 421 |
| | Compound 11 | | | | | |
| Example 8 | 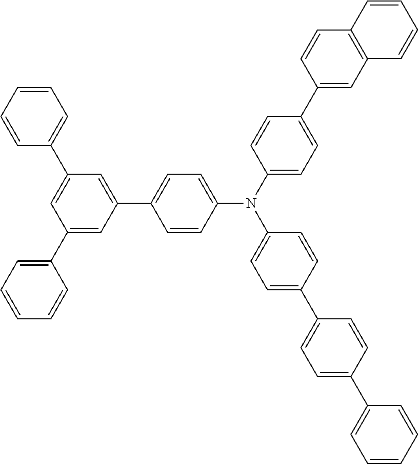 [SMILES: c1ccc(-c2ccc(-c3ccc(N(c4ccc(-c5cc(-c6ccccc6)cc(-c6ccccc6)c5)cc4)c4ccc(-c5ccc6ccccc6c5)cc4)cc3)cc2)cc1] | 3.40 | 7.5 | 0.045 | 166.7 | 414 |
| | Compound 8 | | | | | |
| Example 9 | 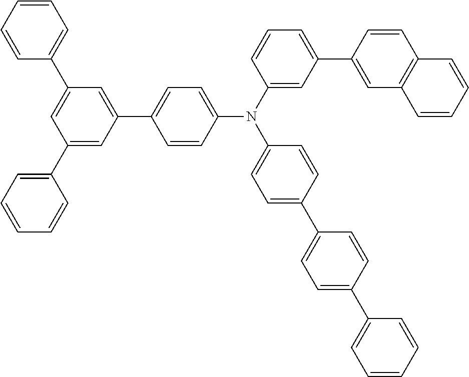 [SMILES: c1ccc(-c2ccc(-c3ccc(N(c4ccc(-c5cc(-c6ccccc6)cc(-c6ccccc6)c5)cc4)c4cccc(-c5ccc6ccccc6c5)c4)cc3)cc2)cc1] | 3.38 | 7.8 | 0.045 | 173.3 | 425 |
| | Compound 10 | | | | | |
| Example 10 | 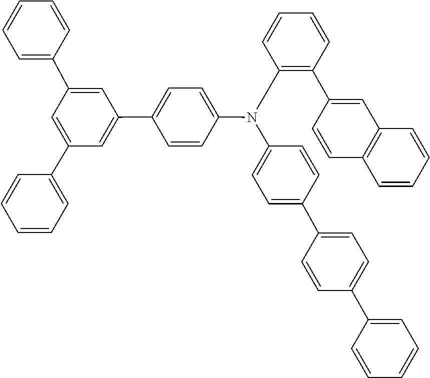 [SMILES: c1ccc(-c2ccc(-c3ccc(N(c4ccc(-c5cc(-c6ccccc6)cc(-c6ccccc6)c5)cc4)c4ccccc4-c4ccc5ccccc5c4)cc3)cc2)cc1] | 3.39 | 7.7 | 0.045 | 171.1 | 419 |
| | Compound 12 | | | | | |
| Example 11 | 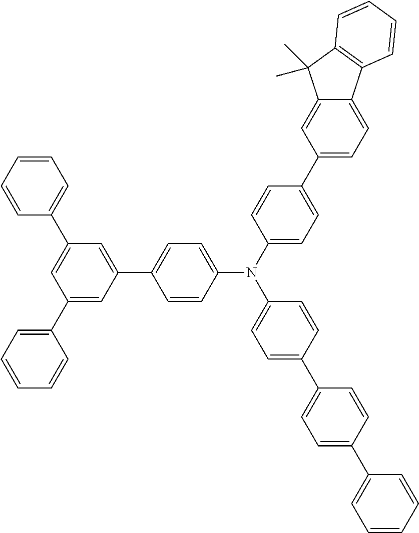 [SMILES: CC1(C)c2ccccc2-c2ccc(-c3ccc(N(c4ccc(-c5ccc(-c6ccccc6)cc5)cc4)c4ccc(-c5cc(-c6ccccc6)cc(-c6ccccc6)c5)cc4)cc3)cc21] | 3.45 | 6.9 | 0.045 | 153.3 | 392 |
| | Compound 23 | | | | | |
| Example 12 | 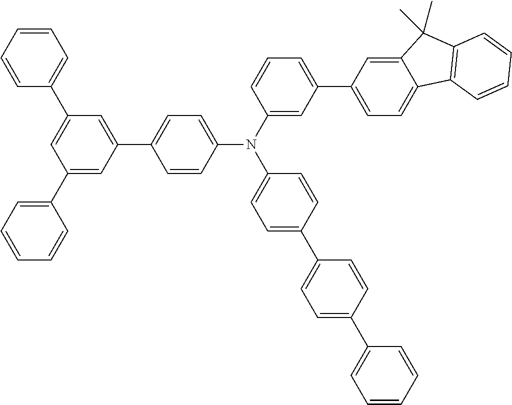 [SMILES: CC1(C)c2ccccc2-c2ccc(-c3cccc(N(c4ccc(-c5ccc(-c6ccccc6)cc5)cc4)c4ccc(-c5cc(-c6ccccc6)cc(-c6ccccc6)c5)cc4)c3)cc21] | 3.42 | 7.2 | 0.045 | 160.0 | 403 |
| | Compound 30 | | | | | |
| Example 13 | 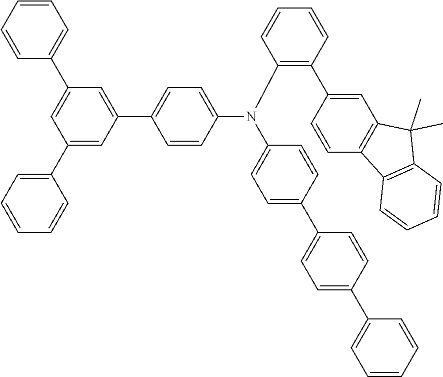 [SMILES: CC1(C)c2ccccc2-c2ccc(-c3ccccc3N(c3ccc(-c4ccc(-c5ccccc5)cc4)cc3)c3ccc(-c4cc(-c5ccccc5)cc(-c5ccccc5)c4)cc3)cc21] | 3.43 | 7.0 | 0.045 | 155.6 | 398 |
| | Compound 34 | | | | | |
| Example 14 | 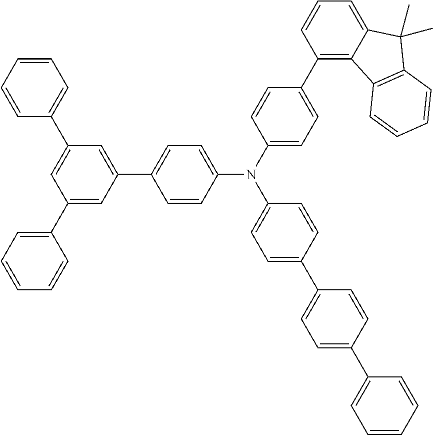 [SMILES: CC1(C)c2ccccc2-c2c(-c3ccc(N(c4ccc(-c5ccc(-c6ccccc6)cc5)cc4)c4ccc(-c5cc(-c6ccccc6)cc(-c6ccccc6)c5)cc4)cc3)cccc21] | 3.45 | 6.7 | 0.045 | 148.9 | 390 |
| | Compound 21 | | | | | |
| Example 15 | 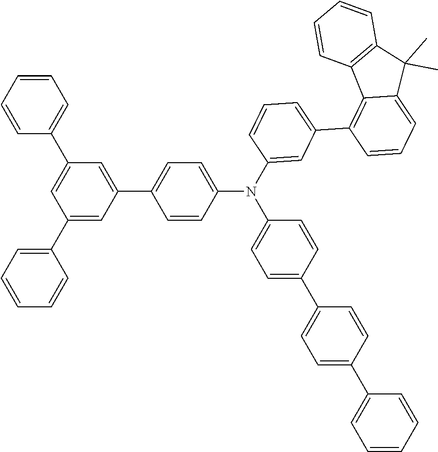 [SMILES: CC1(C)c2ccccc2-c2c(-c3cccc(N(c4ccc(-c5ccc(-c6ccccc6)cc5)cc4)c4ccc(-c5cc(-c6ccccc6)cc(-c6ccccc6)c5)cc4)c3)cccc21] | 3.42 | 7.1 | 0.045 | 157.8 | 401 |
| | Compound 28 | | | | | |
| Example 16 | 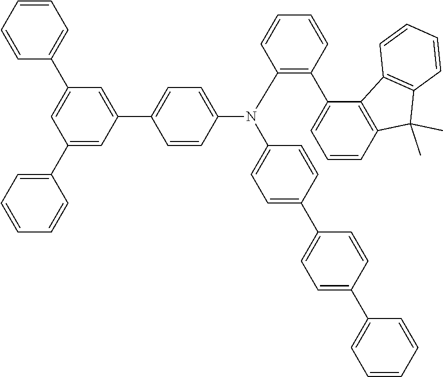 [SMILES: CC1(C)c2ccccc2-c2c(-c3ccccc3N(c3ccc(-c4ccc(-c5ccccc5)cc4)cc3)c3ccc(-c4cc(-c5ccccc5)cc(-c5ccccc5)c4)cc3)cccc21] | 3.43 | 6.9 | 0.045 | 153.3 | 396 |
| | Compound 32 | | | | | |
| Example 17 | 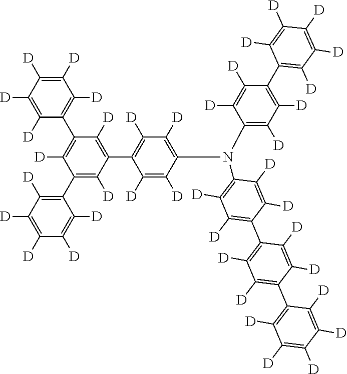 [SMILES: [2H]c1c([2H])c([2H])c(-c2c([2H])c([2H])c(-c3c([2H])c([2H])c(N(c4c([2H])c([2H])c(-c5c([2H])c([2H])c([2H])c([2H])c5[2H])c([2H])c4[2H])c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c([2H])c3[2H])c([2H])c2[2H])c([2H])c1[2H]] | 3.42 | 8.3 | 0.045 | 184.4 | 478 |
| | Compound 38 | | | | | |
| Example 18 | 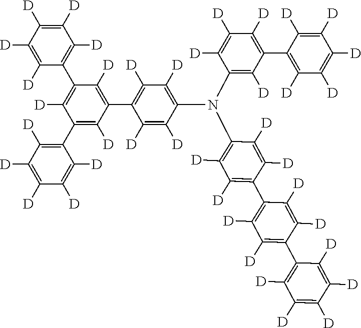 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c([2H])c5[2H])c4[2H])c([2H])c3[2H])c([2H])c2[2H])c([2H])c1[2H]] | 3.41 | 8.5 | 0.045 | 188.9 | 505 |
| | Compound 41 | | | | | |
| Example 19 | 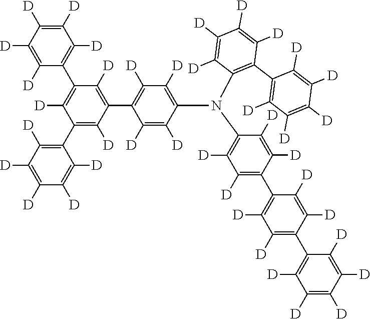 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c([2H])c4-c4c([2H])c([2H])c([2H])c([2H])c4[2H])c([2H])c3[2H])c([2H])c2[2H])c([2H])c1[2H]] | 3.41 | 8.4 | 0.045 | 186.7 | 482 |
| | Compound 44 | | | | | |
| Example 20 | 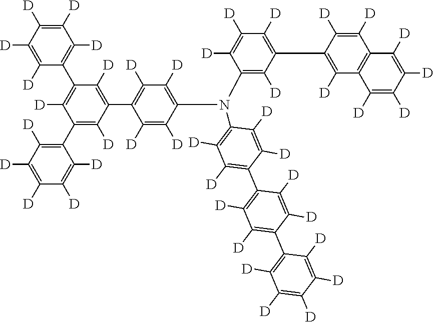 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | 3.45 | 7.8 | 0.045 | 173.3 | 467 |
| | Compound 47 | | | | | |
| Comp. Example 1 | 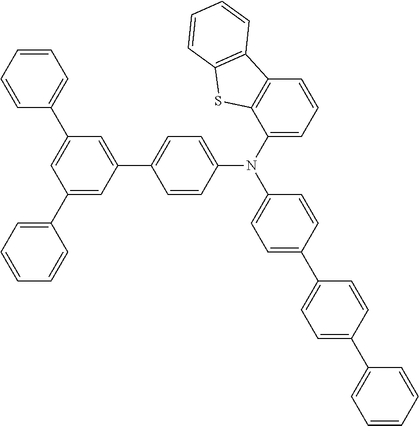 [SMILES: c1ccc(-c2ccc(-c3ccc(N(c4ccc(-c5cc(-c6ccccc6)cc(-c6ccccc6)c5)cc4)c4cccc5c4sc4ccccc45)cc3)cc2)cc1] | 3.63 | 6.3 | 0.045 | 140.0 | 351 |
| | Comparative Compound 1 | | | | | |
| Comp. Example 2 | 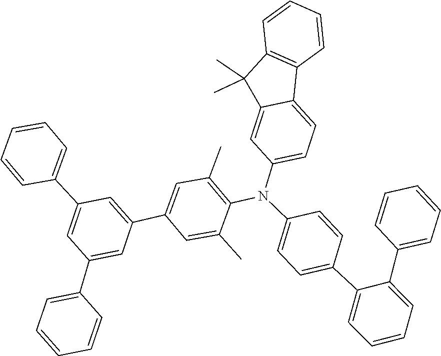 [SMILES: Cc1cc(-c2cc(-c3ccccc3)cc(-c3ccccc3)c2)cc(C)c1N(c1ccc(-c2ccccc2-c2ccccc2)cc1)c1ccc2c(c1)C(C)(C)c1ccccc1-2] | 3.57 | 5.9 | 0.045 | 131.1 | 329 |
| | Comparative Compound 2 | | | | | |
| Comp. Example 3 | 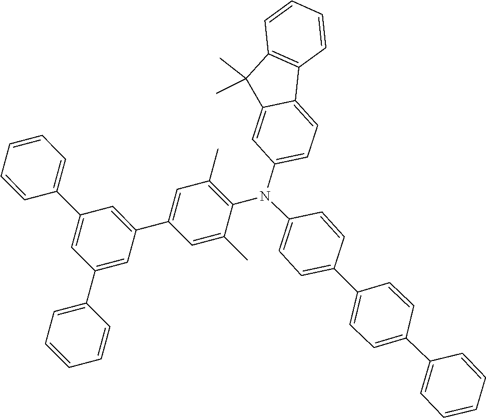 [SMILES: Cc1cc(-c2cc(-c3ccccc3)cc(-c3ccccc3)c2)cc(C)c1N(c1ccc(-c2ccc(-c3ccccc3)cc2)cc1)c1ccc2c(c1)C(C)(C)c1ccccc1-2] | 3.56 | 6.0 | 0.045 | 133.3 | 341 |
| | Comparative Compound 3 | | | | | |
| Comp. Example 4 | 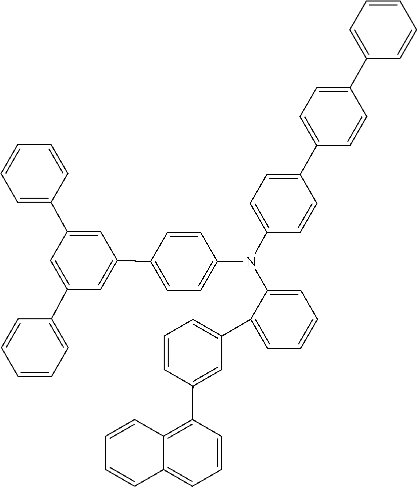 [SMILES: c1ccc(-c2ccc(-c3ccc(N(c4ccc(-c5cc(-c6ccccc6)cc(-c6ccccc6)c5)cc4)c4ccccc4-c4cccc(-c5cccc6ccccc56)c4)cc3)cc2)cc1] | 3.55 | 6.0 | 0.045 | 133.3 | 305 |
| | Comparative Compound 4 | | | | | |
| Comp. Example 5 | 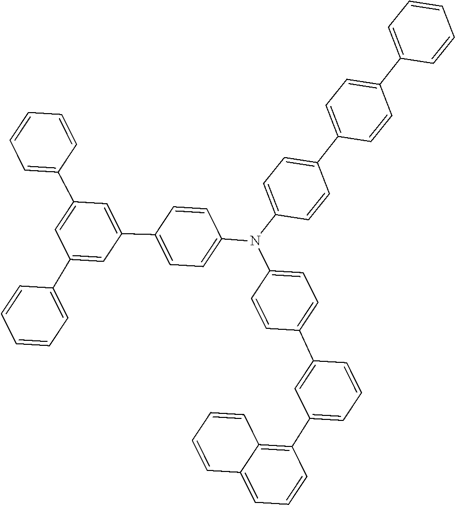 [SMILES: c1ccc(-c2ccc(-c3ccc(N(c4ccc(-c5cc(-c6ccccc6)cc(-c6ccccc6)c5)cc4)c4ccc(-c5cccc(-c6cccc7ccccc67)c5)cc4)cc3)cc2)cc1] | 3.56 | 6.1 | 0.045 | 135.6 | 312 |
| | Comparative Compound 5 | | | | | |
| Comp. Example 6 | 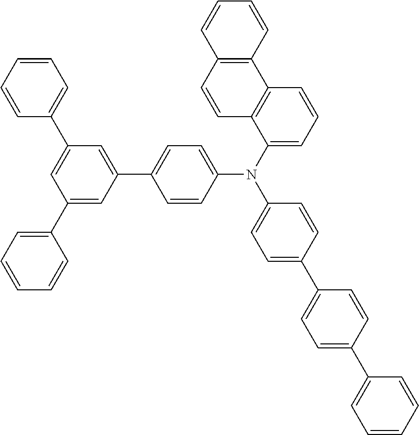 [SMILES: c1ccc(-c2ccc(-c3ccc(N(c4ccc(-c5cc(-c6ccccc6)cc(-c6ccccc6)c5)cc4)c4cccc5c4ccc4ccccc45)cc3)cc2)cc1] | 3.61 | 6.2 | 0.045 | 137.8 | 343 |
| | Comparative Compound 6 | | | | | |

Referring to Table 2, it was confirmed that when Comparative Compounds 1 to 6 were used, the driving voltage increased, the efficiency and lifetime decreased, and the BI value also decreased in the organic light emitting diode evaluation results, compared to the cases where the compounds represented by Chemical Formula A according to the present disclosure were used.

Comparative Compound 1 differs structurally from Chemical Formula A according to the present disclosure in that Ar is a heteroaryl group instead of an aryl group. Comparative Compound 2 structurally differs from Chemical Formula A in that its bonding relationship is different from the structure of terphenyl in which three phenyl groups are bonded in the para-position in Chemical Formula A according to the present disclosure. Additionally, compared to Chemical Formula A, Comparative Compounds 2, 3, and 6 have a structure in which the aryl group is directly bonded to Ar without a linker. Comparative Compounds 4 and 5 correspond to structures in which the linker connecting the Ar structure to the aryl group is excessively long, compared to Chemical Formula A. That is, it was confirmed that the Example compounds having the Ar structure described in claim 1 of the present disclosure exhibit superior organic light emitting diode performance and improved BI values compared to Comparative Compounds 1 to 6, which do not have the structure.

### [Experimental Example 2] HOMO and LUMO Measurement—Hole Transport Auxiliary Layer Simulation Results

The hole transport auxiliary layer serves to reduce the accumulation of holes at the interface of the emitting layer caused by a difference in HOMO energy levels between the hole transport layer and the emitting layer. For this purpose, it is preferable that the HOMO energy level difference between the hole transport auxiliary layer and the emitting layer is smaller than the HOMO energy level difference between the hole transport auxiliary layer and the hole transport layer. In addition, in order to minimize electrons coming from the emitting layer to the hole transport layer, the hole transport auxiliary layer preferably has a LUMO energy level higher than that of the emitting layer.

In order to confirm whether the compounds represented by Chemical Formula A according to the present disclosure are suitable as hole transport auxiliary layer materials, the HOMO energy levels (eV) and LUMO energy levels (eV) were calculated for the compounds in Table 3 below using Gaussian software (B3LYP DFT 6-31G* by Gaussian 16), and the results are shown in Table 3 below. It was confirmed that the compounds represented by Chemical Formula A according to the present disclosure are suitable as hole transport auxiliary layer materials by satisfying a HOMO range of −4.80 eV to −5.00 eV, while Comparative Compounds 2 and 3 were found to be unsuitable as hole transport auxiliary layer materials as they did not satisfy the above HOMO range.

TABLE 3

| Compounds of | HOMO | LUMO |
|---|---|---|
| Examples/ | (calculation) | (calculation) |
| Comp. Examples | (eV) | (eV) |
| 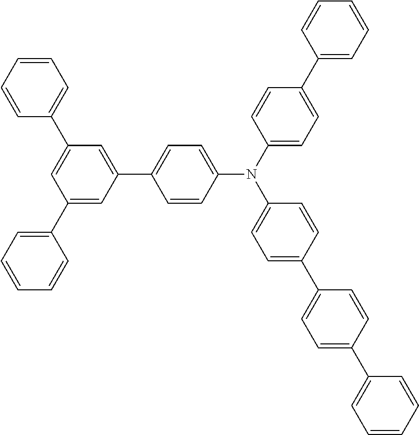 [SMILES: c1ccc(-c2ccc(-c3ccc(N(c4ccc(-c5ccccc5)cc4)c4ccc(-c5cc(-c6ccccc6)cc(-c6ccccc6)c5)cc4)cc3)cc2)cc1] | −4.96 | −0.86 |
| Compound 4 | | |
| 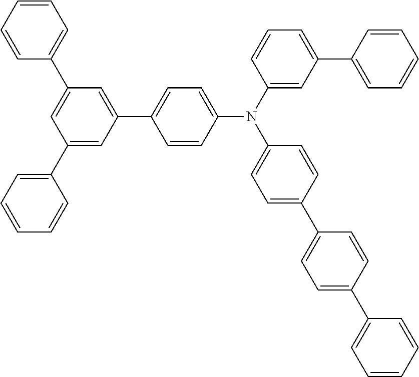 [SMILES: c1ccc(-c2ccc(-c3ccc(N(c4ccc(-c5cc(-c6ccccc6)cc(-c6ccccc6)c5)cc4)c4cccc(-c5ccccc5)c4)cc3)cc2)cc1] | −4.99 | −0.85 |
| Compound 5 | | |
| 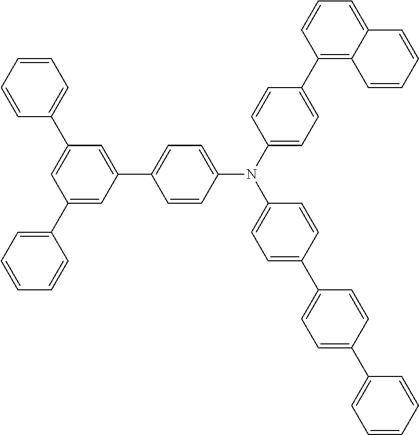 [SMILES: c1ccc(-c2ccc(-c3ccc(N(c4ccc(-c5cc(-c6ccccc6)cc(-c6ccccc6)c5)cc4)c4ccc(-c5cccc6ccccc56)cc4)cc3)cc2)cc1] | −4.99 | −1.11 |
| Compound 7 | | |
| 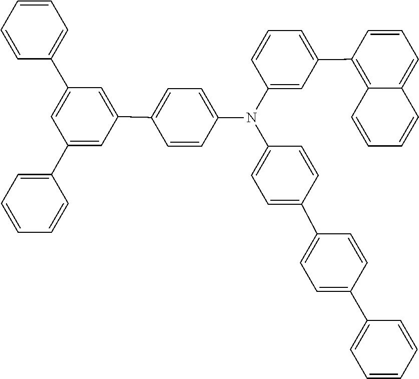 [SMILES: c1ccc(-c2ccc(-c3ccc(N(c4ccc(-c5cc(-c6ccccc6)cc(-c6ccccc6)c5)cc4)c4cccc(-c5cccc6ccccc56)c4)cc3)cc2)cc1] | −4.99 | −1.14 |
| Compound 9 | | |
| 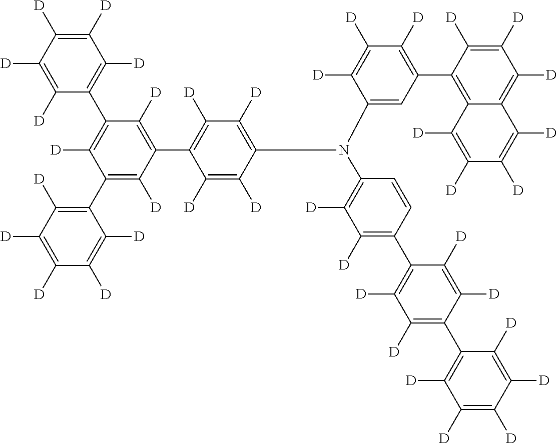 [SMILES: [2H]c1c(-c2c([2H])c([2H])c(-c3c([2H])c([2H])c([2H])c([2H])c3[2H])c([2H])c2[2H])ccc(N(c2cc(-c3c([2H])c([2H])c([2H])c4c([2H])c([2H])c([2H])c([2H])c34)c([2H])c([2H])c2[2H])c2c([2H])c([2H])c(-c3c([2H])c(-c4c([2H])c([2H])c([2H])c([2H])c4[2H])c([2H])c(-c4c([2H])c([2H])c([2H])c([2H])c4[2H])c3[2H])c([2H])c2[2H])c1[2H]] | −4.99 | −1.14 |
| Compound 27 | | |
| 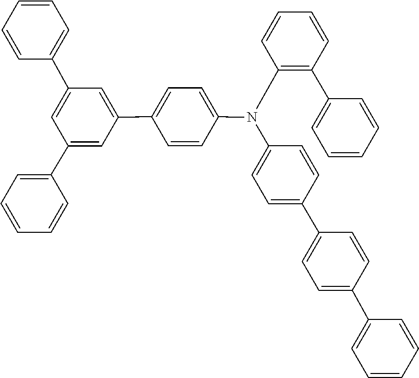 [SMILES: c1ccc(-c2ccc(-c3ccc(N(c4ccc(-c5cc(-c6ccccc6)cc(-c6ccccc6)c5)cc4)c4ccccc4-c4ccccc4)cc3)cc2)cc1] | −4.97 | −0.99 |
| Compound 6 | | |
| 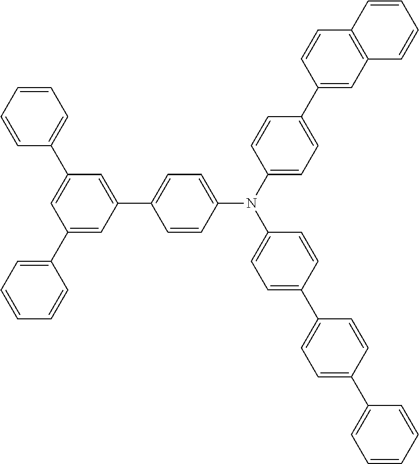 [SMILES: c1ccc(-c2ccc(-c3ccc(N(c4ccc(-c5cc(-c6ccccc6)cc(-c6ccccc6)c5)cc4)c4ccc(-c5ccc6ccccc6c5)cc4)cc3)cc2)cc1] | −4.88 | −1.22 |
| Compound 8 | | |
| 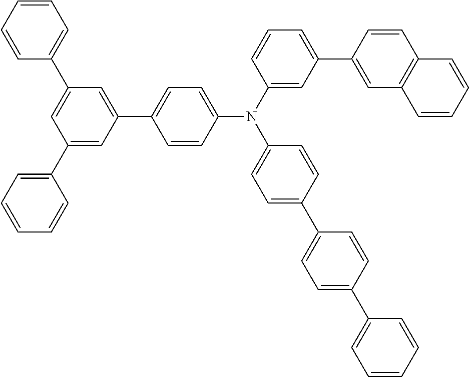 [SMILES: c1ccc(-c2ccc(-c3ccc(N(c4ccc(-c5cc(-c6ccccc6)cc(-c6ccccc6)c5)cc4)c4cccc(-c5ccc6ccccc6c5)c4)cc3)cc2)cc1] | −4.93 | −1.18 |
| Compound 10 | | |
| 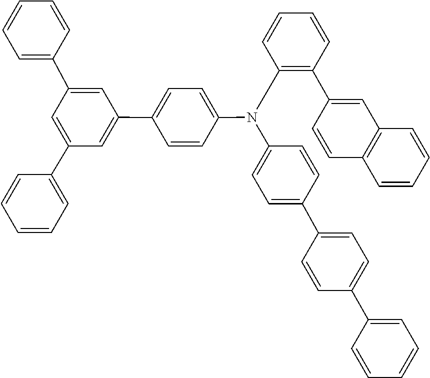 [SMILES: c1ccc(-c2ccc(-c3ccc(N(c4ccc(-c5cc(-c6ccccc6)cc(-c6ccccc6)c5)cc4)c4ccccc4-c4ccc5ccccc5c4)cc3)cc2)cc1] | −4.90 | −1.16 |
| Compound 12 | | |
| 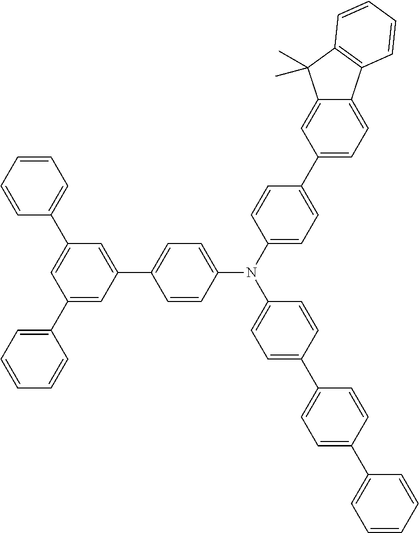 [SMILES: CC1(C)c2ccccc2-c2ccc(-c3ccc(N(c4ccc(-c5ccc(-c6ccccc6)cc5)cc4)c4ccc(-c5cc(-c6ccccc6)cc(-c6ccccc6)c5)cc4)cc3)cc21] | −4.85 | −1.17 |
| Compound 23 | | |
| 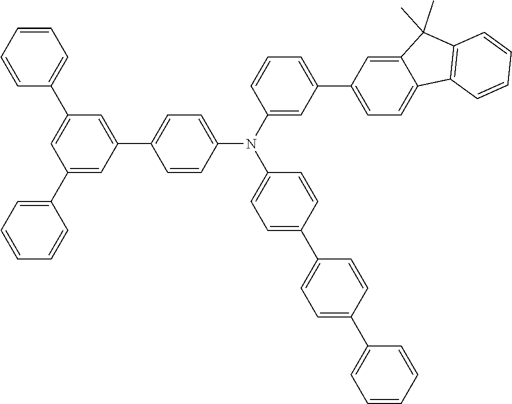 [SMILES: CC1(C)c2ccccc2-c2ccc(-c3cccc(N(c4ccc(-c5ccc(-c6ccccc6)cc5)cc4)c4ccc(-c5cc(-c6ccccc6)cc(-c6ccccc6)c5)cc4)c3)cc21] | −4.91 | −1.12 |
| Compound 30 | | |
| 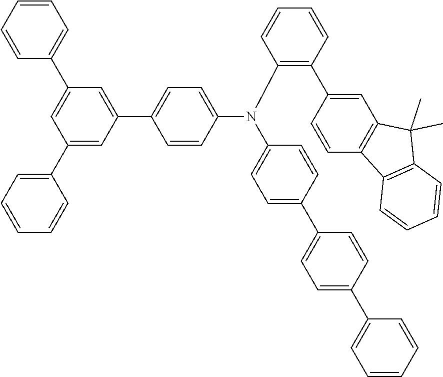 [SMILES: CC1(C)c2ccccc2-c2ccc(-c3ccccc3N(c3ccc(-c4ccc(-c5ccccc5)cc4)cc3)c3ccc(-c4cc(-c5ccccc5)cc(-c5ccccc5)c4)cc3)cc21] | −4.89 | −1.05 |
| Compound 34 | | |
| 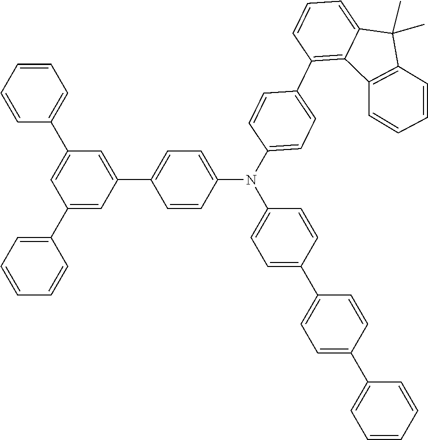 [SMILES: CC1(C)c2ccccc2-c2c(-c3ccc(N(c4ccc(-c5ccc(-c6ccccc6)cc5)cc4)c4ccc(-c5cc(-c6ccccc6)cc(-c6ccccc6)c5)cc4)cc3)cccc21] | −4.86 | −1.08 |
| Compound 21 | | |
| 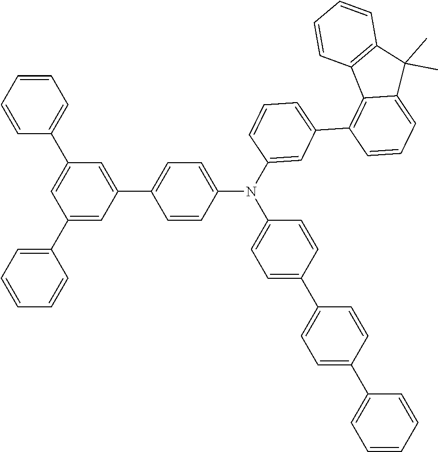 [SMILES: CC1(C)c2ccccc2-c2c(-c3cccc(N(c4ccc(-c5ccc(-c6ccccc6)cc5)cc4)c4ccc(-c5cc(-c6ccccc6)cc(-c6ccccc6)c5)cc4)c3)cccc21] | −4.92 | −1.11 |
| Compound 28 | | |
| 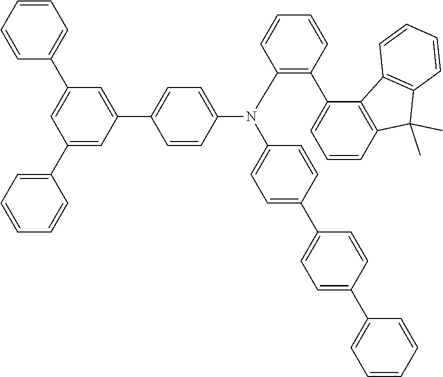 [SMILES: CC1(C)c2ccccc2-c2c(-c3ccccc3N(c3ccc(-c4ccc(-c5ccccc5)cc4)cc3)c3ccc(-c4cc(-c5ccccc5)cc(-c5ccccc5)c4)cc3)cccc21] | −4.89 | −1.04 |
| Compound 32 | | |
| 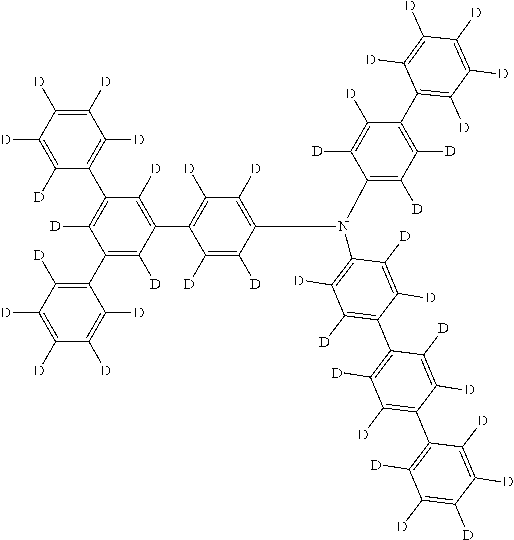 [SMILES: [2H]c1c([2H])c([2H])c(-c2c([2H])c([2H])c(-c3c([2H])c([2H])c(N(c4c([2H])c([2H])c(-c5c([2H])c([2H])c([2H])c([2H])c5[2H])c([2H])c4[2H])c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c([2H])c3[2H])c([2H])c2[2H])c([2H])c1[2H]] | −4.96 | −0.86 |
| Compound 38 | | |
| 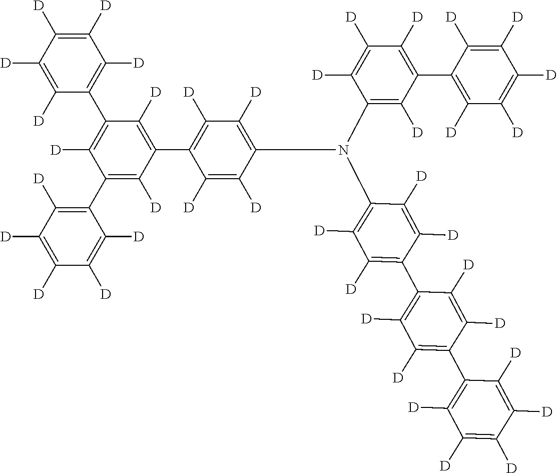 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c([2H])c5[2H])c4[2H])c([2H])c3[2H])c([2H])c2[2H])c([2H])c1[2H]] | −4.99 | −0.85 |
| Compound 41 | | |
| 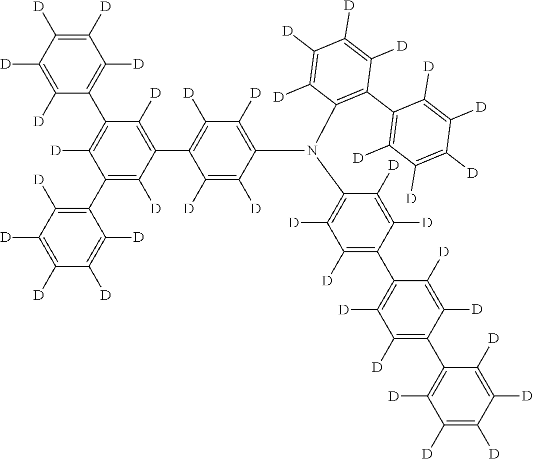 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c([2H])c4-c4c([2H])c([2H])c([2H])c([2H])c4[2H])c([2H])c3[2H])c([2H])c2[2H])c([2H])c1[2H]] | −4.97 | −0.99 |
| Compound 44 | | |
| 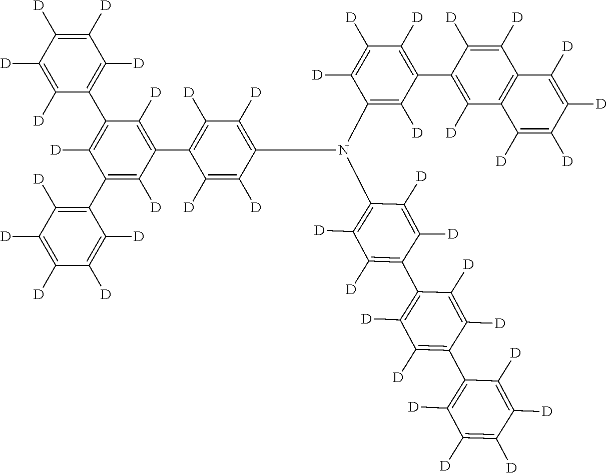 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | −4.92 | −1.18 |
| Compound 47 | | |
| 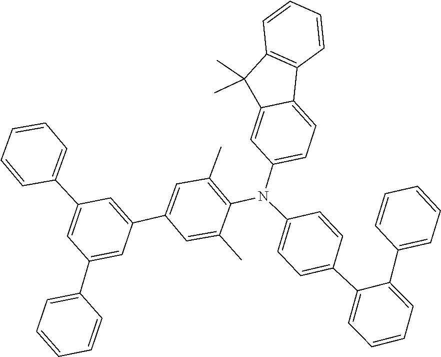 [SMILES: Cc1cc(-c2cc(-c3ccccc3)cc(-c3ccccc3)c2)cc(C)c1N(c1ccc(-c2ccccc2-c2ccccc2)cc1)c1ccc2c(c1)C(C)(C)c1ccccc1-2] | −4.75 | −0.92 |
| Comparative Compound 2 | | |
| 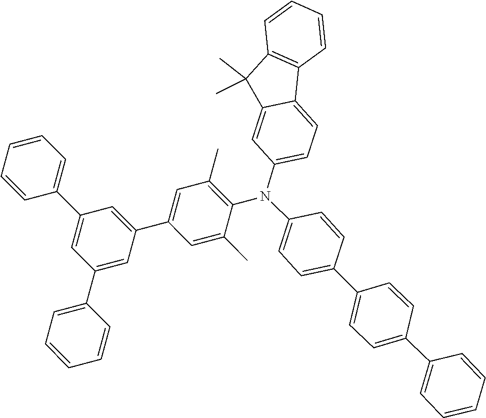 [SMILES: Cc1cc(-c2cc(-c3ccccc3)cc(-c3ccccc3)c2)cc(C)c1N(c1ccc(-c2ccc(-c3ccccc3)cc2)cc1)c1ccc2c(c1)C(C)(C)c1ccccc1-2] | −4.74 | −0.96 |
| Comparative Compound 3 | | |

### [Experimental Example 3] Triplet Energy (T₁) Calculation—Quantum Calculation

Quantum calculation experiments were conducted for the compounds of Table 4 below, which are represented by Chemical Formula A according to the present disclosure, and Comparative Compounds 1 to 6 using Gaussian software (B3LYP DFT 6-31G* by Gaussian 16) to calculate the lowest triplet energy (T₁, absolute value). The results are shown in Table 4 below.

TABLE 4

| Compounds of | T1 |
|---|---|
| Examples/Comp. | (calculation) |
| Examples | (eV) |
| 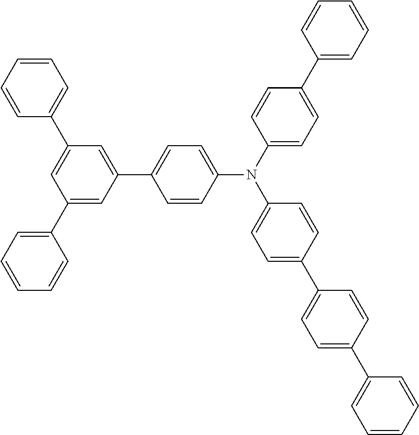 [SMILES: c1ccc(-c2ccc(-c3ccc(N(c4ccc(-c5ccccc5)cc4)c4ccc(-c5cc(-c6ccccc6)cc(-c6ccccc6)c5)cc4)cc3)cc2)cc1] | 2.71 |
| Compound 4 | |
| 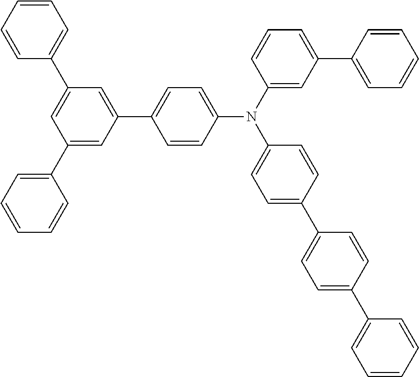 [SMILES: c1ccc(-c2ccc(-c3ccc(N(c4ccc(-c5cc(-c6ccccc6)cc(-c6ccccc6)c5)cc4)c4cccc(-c5ccccc5)c4)cc3)cc2)cc1] | 2.76 |
| Compound 5 | |
| 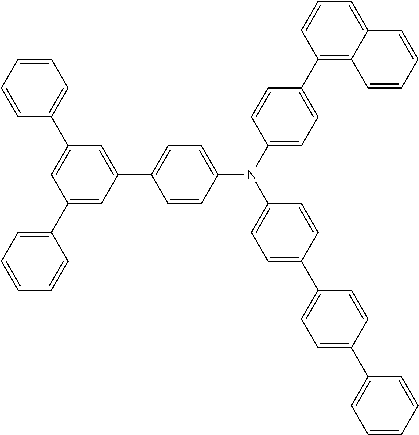 [SMILES: c1ccc(-c2ccc(-c3ccc(N(c4ccc(-c5cc(-c6ccccc6)cc(-c6ccccc6)c5)cc4)c4ccc(-c5cccc6ccccc56)cc4)cc3)cc2)cc1] | 2.63 |
| Compound 7 | |
| 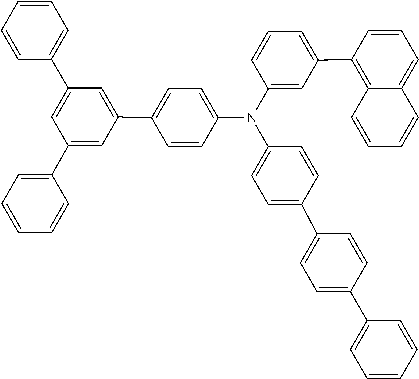 [SMILES: c1ccc(-c2ccc(-c3ccc(N(c4ccc(-c5cc(-c6ccccc6)cc(-c6ccccc6)c5)cc4)c4cccc(-c5cccc6ccccc56)c4)cc3)cc2)cc1] | 2.67 |
| Compound 9 | |
| 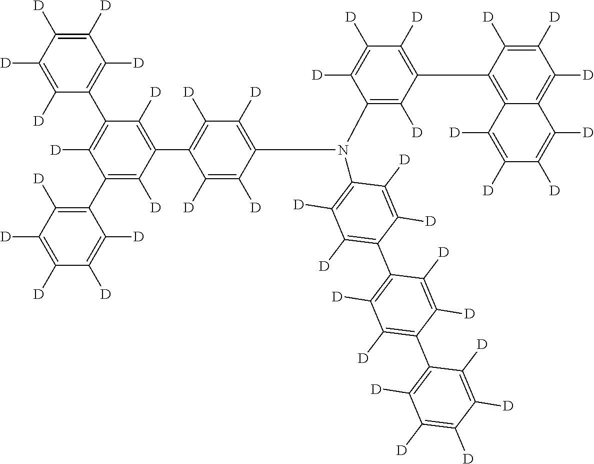 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | 2.62 |
| Compound 27 | |
| 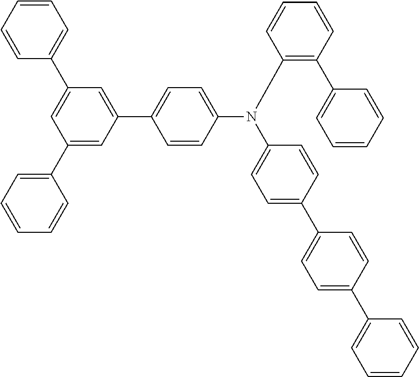 [SMILES: c1ccc(-c2ccc(-c3ccc(N(c4ccc(-c5cc(-c6ccccc6)cc(-c6ccccc6)c5)cc4)c4ccccc4-c4ccccc4)cc3)cc2)cc1] | 2.74 |
| Compound 6 | |
| 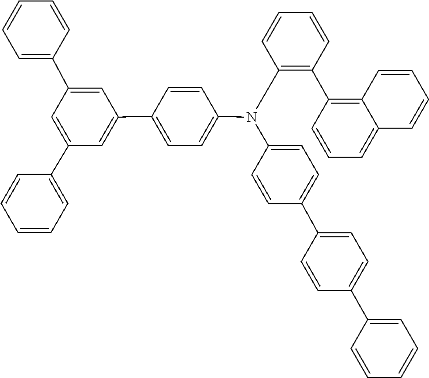 [SMILES: c1ccc(-c2ccc(-c3ccc(N(c4ccc(-c5cc(-c6ccccc6)cc(-c6ccccc6)c5)cc4)c4ccccc4-c4cccc5ccccc45)cc3)cc2)cc1] | 2.65 |
| Compound 11 | |
| 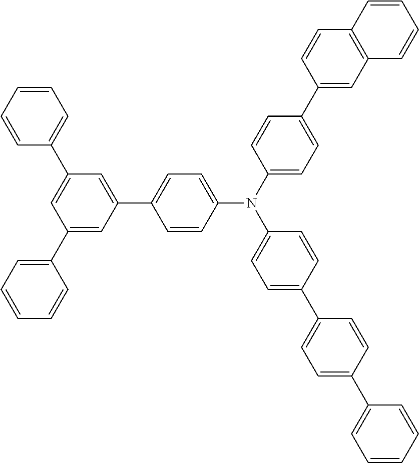 [SMILES: c1ccc(-c2ccc(-c3ccc(N(c4ccc(-c5cc(-c6ccccc6)cc(-c6ccccc6)c5)cc4)c4ccc(-c5ccc6ccccc6c5)cc4)cc3)cc2)cc1] | 2.62 |
| Compound 8 | |
| 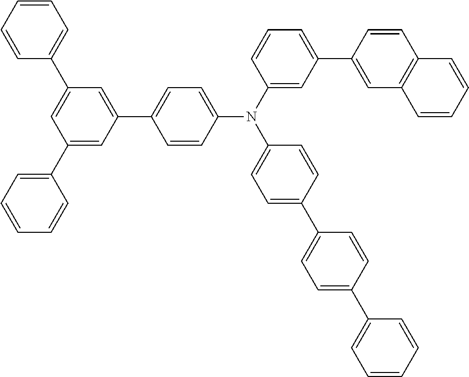 [SMILES: c1ccc(-c2ccc(-c3ccc(N(c4ccc(-c5cc(-c6ccccc6)cc(-c6ccccc6)c5)cc4)c4cccc(-c5ccc6ccccc6c5)c4)cc3)cc2)cc1] | 2.67 |
| Compound 10 | |
| 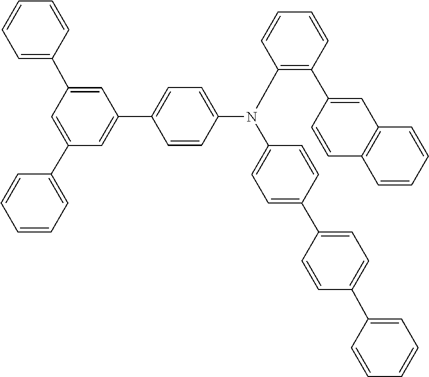 [SMILES: c1ccc(-c2ccc(-c3ccc(N(c4ccc(-c5cc(-c6ccccc6)cc(-c6ccccc6)c5)cc4)c4ccccc4-c4ccc5ccccc5c4)cc3)cc2)cc1] | 2.65 |
| Compound 12 | |
| 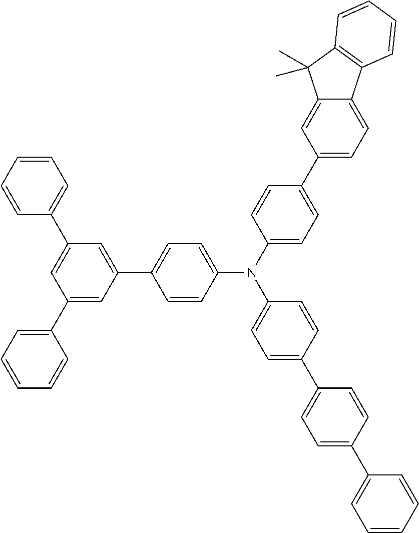 [SMILES: CC1(C)c2ccccc2-c2ccc(-c3ccc(N(c4ccc(-c5ccc(-c6ccccc6)cc5)cc4)c4ccc(-c5cc(-c6ccccc6)cc(-c6ccccc6)c5)cc4)cc3)cc21] | 2.68 |
| Compound 23 | |
| 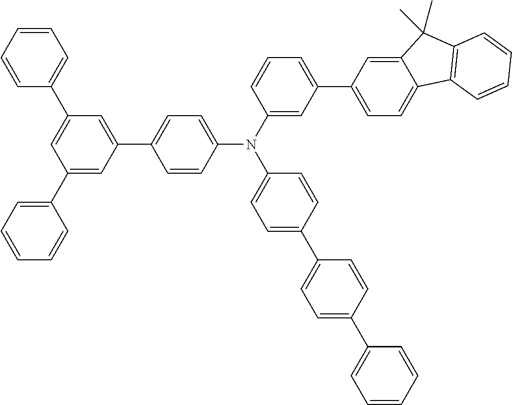 [SMILES: CC1(C)c2ccccc2-c2ccc(-c3cccc(N(c4ccc(-c5ccc(-c6ccccc6)cc5)cc4)c4ccc(-c5cc(-c6ccccc6)cc(-c6ccccc6)c5)cc4)c3)cc21] | 2.72 |
| Compound 30 | |
| 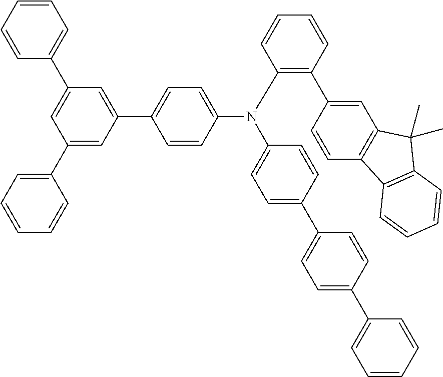 [SMILES: CC1(C)c2ccccc2-c2ccc(-c3ccccc3N(c3ccc(-c4ccc(-c5ccccc5)cc4)cc3)c3ccc(-c4cc(-c5ccccc5)cc(-c5ccccc5)c4)cc3)cc21] | 2.70 |
| Compound 34 | |
| 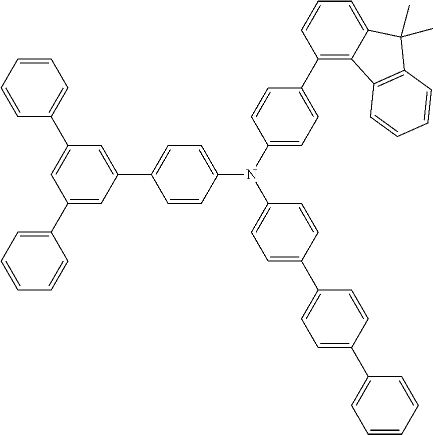 [SMILES: CC1(C)c2ccccc2-c2c(-c3ccc(N(c4ccc(-c5ccc(-c6ccccc6)cc5)cc4)c4ccc(-c5cc(-c6ccccc6)cc(-c6ccccc6)c5)cc4)cc3)cccc21] | 2.66 |
| Compound 21 | |
| 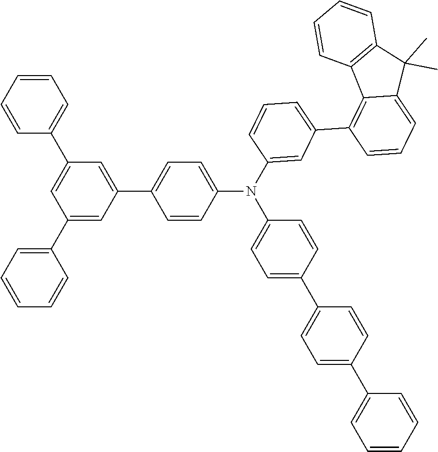 [SMILES: CC1(C)c2ccccc2-c2c(-c3cccc(N(c4ccc(-c5ccc(-c6ccccc6)cc5)cc4)c4ccc(-c5cc(-c6ccccc6)cc(-c6ccccc6)c5)cc4)c3)cccc21] | 2.71 |
| Compound 28 | |
| 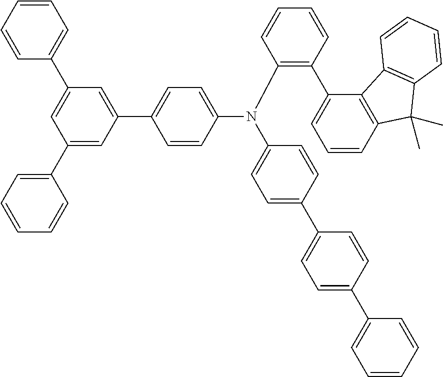 [SMILES: CC1(C)c2ccccc2-c2c(-c3ccccc3N(c3ccc(-c4ccc(-c5ccccc5)cc4)cc3)c3ccc(-c4cc(-c5ccccc5)cc(-c5ccccc5)c4)cc3)cccc21] | 2.69 |
| Compound 32 | |
| 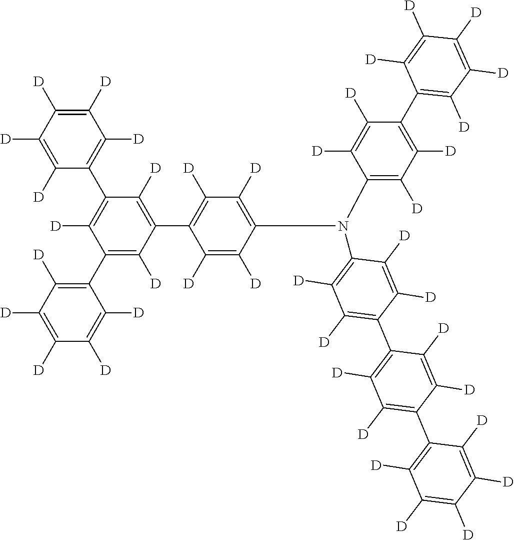 [SMILES: [2H]c1c([2H])c([2H])c(-c2c([2H])c([2H])c(-c3c([2H])c([2H])c(N(c4c([2H])c([2H])c(-c5c([2H])c([2H])c([2H])c([2H])c5[2H])c([2H])c4[2H])c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c([2H])c3[2H])c([2H])c2[2H])c([2H])c1[2H]] | 2.68 |
| Compound 38 | |
| 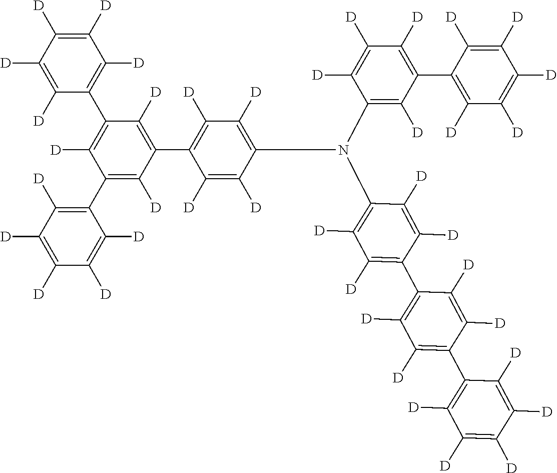 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c([2H])c5[2H])c4[2H])c([2H])c3[2H])c([2H])c2[2H])c([2H])c1[2H]] | 2.69 |
| Compound 41 | |
| 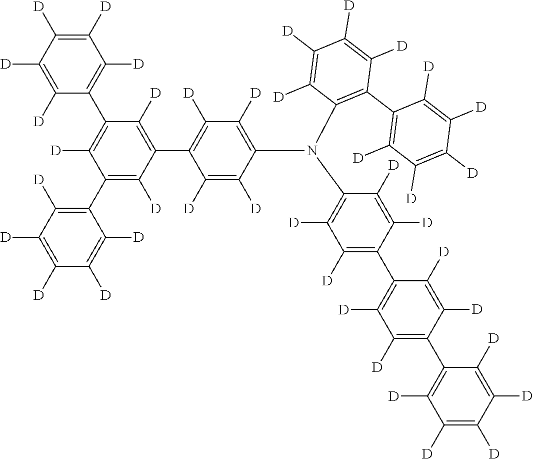 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c([2H])c4-c4c([2H])c([2H])c([2H])c([2H])c4[2H])c([2H])c3[2H])c([2H])c2[2H])c([2H])c1[2H]] | 2.71 |
| Compound 44 | |
| 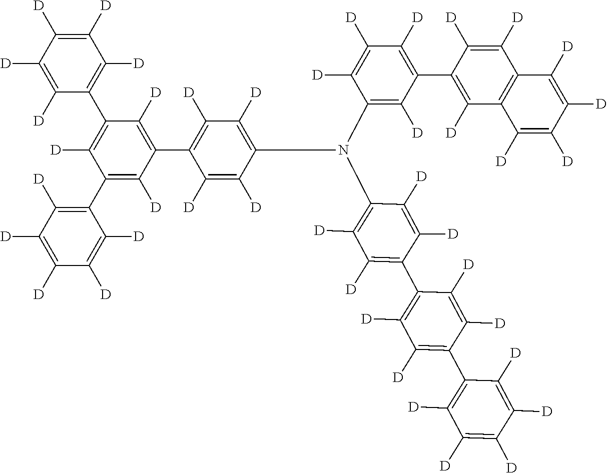 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | 2.62 |
| Compound 47 | |
| 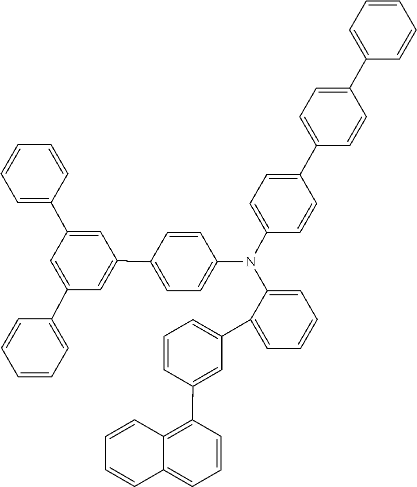 [SMILES: c1ccc(-c2ccc(-c3ccc(N(c4ccc(-c5cc(-c6ccccc6)cc(-c6ccccc6)c5)cc4)c4ccccc4-c4cccc(-c5cccc6ccccc56)c4)cc3)cc2)cc1] | 2.57 |
| Comparative Compound 4 | |
| 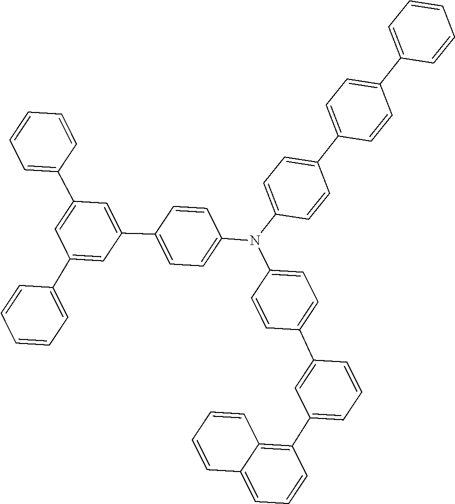 [SMILES: c1ccc(-c2ccc(-c3ccc(N(c4ccc(-c5cc(-c6ccccc6)cc(-c6ccccc6)c5)cc4)c4ccc(-c5cccc(-c6cccc7ccccc67)c5)cc4)cc3)cc2)cc1] | 2.59 |
| Comparative Compound 5 | |
| 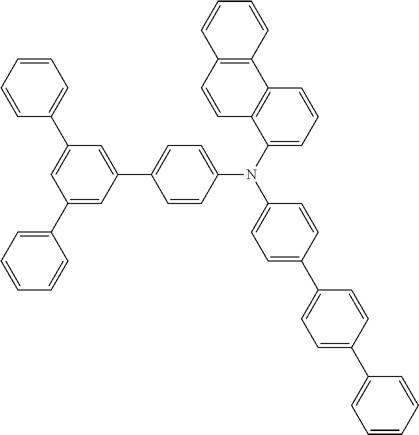 [SMILES: c1ccc(-c2ccc(-c3ccc(N(c4ccc(-c5cc(-c6ccccc6)cc(-c6ccccc6)c5)cc4)c4cccc5c4ccc4ccccc45)cc3)cc2)cc1] | 2.56 |
| Comparative Compound 6 | |

Referring to Table 4 above, the organic compound represented by Chemical Formula A according to the present disclosure has a triplet energy level (T₁) of 2.60 to 2.80 eV, which is an appropriate range for use a hole transport layer or a hole transport auxiliary layer, thereby effectively suppressing diffusion of triplet excitons generated in the emitting layer to the outside and improving light-emitting efficiency and organic light emitting diode lifetime characteristics.

Meanwhile, when the triplet energy level is excessively high, the HOMO energy level becomes deeper; accordingly, hole transport properties may deteriorate, or the driving voltage of the organic light emitting diode may increase.

Conversely, when the triplet energy level is excessively low, the diffusion of triplet excitons generated in the emitting layer cannot be prevented, thereby leading to a decline in organic light emitting diode efficiency.

Accordingly, the organic compound represented by Chemical Formula A according to the present disclosure may have a triplet energy level within a range that ensures a balance between a triplet exciton confinement effect and charge transport properties.

### [Experimental Example 4] Deposition Temperature (Te) Measurement

The deposition temperatures (Te) of the compounds represented by Chemical Formula A according to the present disclosure and Comparative Compounds 1 to 6 were measured, and the results are shown in Table 5 below. Here, the deposition temperature refers to the temperature (° C.) at which the weight of the compound decreases by 10% through sublimation under a pressure condition of 10 Pa, as measured by a thermogravimetric analysis (TGA) method.

TABLE 5

| Compounds of | Molecular | |
|---|---|---|
| Examples/Comp. | Weight | Te |
| Examples | (Mw) | (° C.) |
| 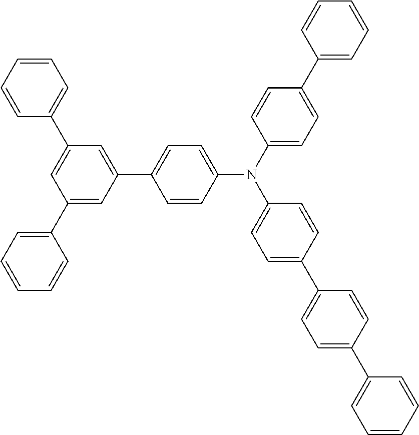 [SMILES: c1ccc(-c2ccc(-c3ccc(N(c4ccc(-c5ccccc5)cc4)c4ccc(-c5cc(-c6ccccc6)cc(-c6ccccc6)c5)cc4)cc3)cc2)cc1] | 701.91 | 300 |
| Compound 4 | | |
| 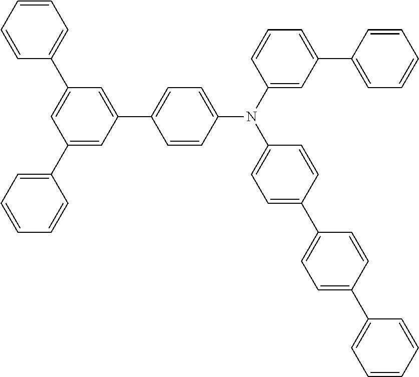 [SMILES: c1ccc(-c2ccc(-c3ccc(N(c4ccc(-c5cc(-c6ccccc6)cc(-c6ccccc6)c5)cc4)c4cccc(-c5ccccc5)c4)cc3)cc2)cc1] | 701.91 | 290 |
| Compound 5 | | |
| 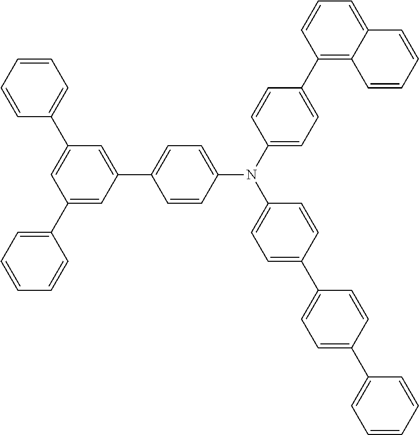 [SMILES: c1ccc(-c2ccc(-c3ccc(N(c4ccc(-c5cc(-c6ccccc6)cc(-c6ccccc6)c5)cc4)c4ccc(-c5cccc6ccccc56)cc4)cc3)cc2)cc1] | 751.97 | 339 |
| Compound 7 | | |
| 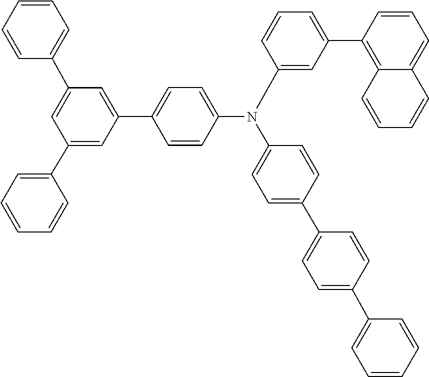 [SMILES: c1ccc(-c2ccc(-c3ccc(N(c4ccc(-c5cc(-c6ccccc6)cc(-c6ccccc6)c5)cc4)c4cccc(-c5cccc6ccccc56)c4)cc3)cc2)cc1] | 751.97 | 330 |
| Compound 9 | | |
| 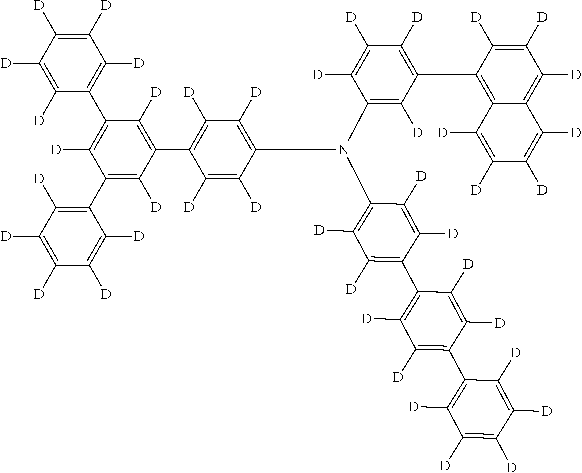 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | 793.22 | 335 |
| Compound 27 | | |
| 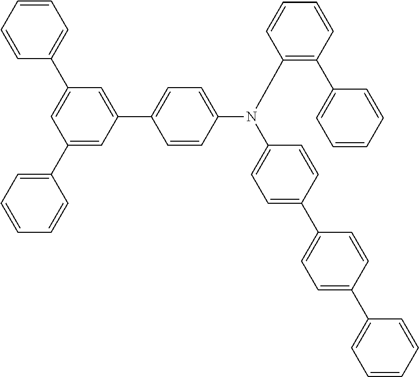 [SMILES: c1ccc(-c2ccc(-c3ccc(N(c4ccc(-c5cc(-c6ccccc6)cc(-c6ccccc6)c5)cc4)c4ccccc4-c4ccccc4)cc3)cc2)cc1] | 701.91 | 294 |
| Compound 6 | | |
| 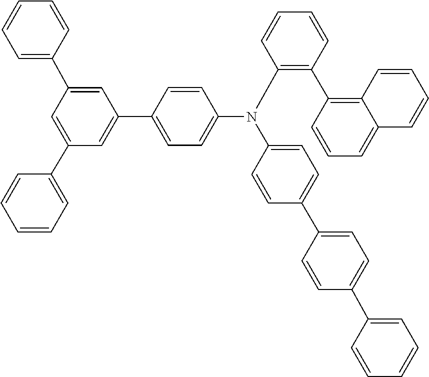 [SMILES: c1ccc(-c2ccc(-c3ccc(N(c4ccc(-c5cc(-c6ccccc6)cc(-c6ccccc6)c5)cc4)c4ccccc4-c4cccc5ccccc45)cc3)cc2)cc1] | 751.97 | 335 |
| Compound 11 | | |
| 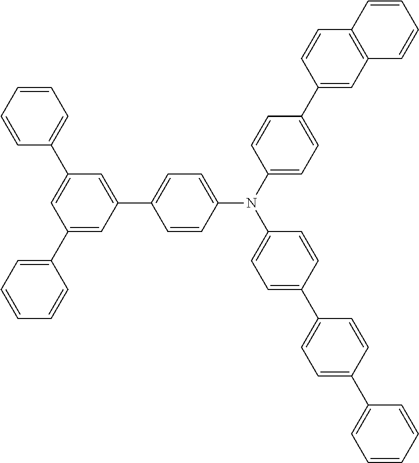 [SMILES: c1ccc(-c2ccc(-c3ccc(N(c4ccc(-c5cc(-c6ccccc6)cc(-c6ccccc6)c5)cc4)c4ccc(-c5ccc6ccccc6c5)cc4)cc3)cc2)cc1] | 751.97 | 340 |
| Compound 8 | | |
| 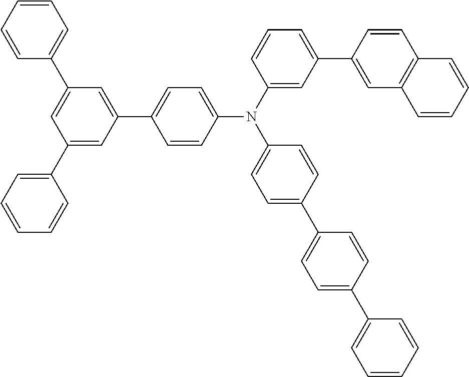 [SMILES: c1ccc(-c2ccc(-c3ccc(N(c4ccc(-c5cc(-c6ccccc6)cc(-c6ccccc6)c5)cc4)c4cccc(-c5ccc6ccccc6c5)c4)cc3)cc2)cc1] | 751.97 | 330 |
| Compound 10 | | |
| 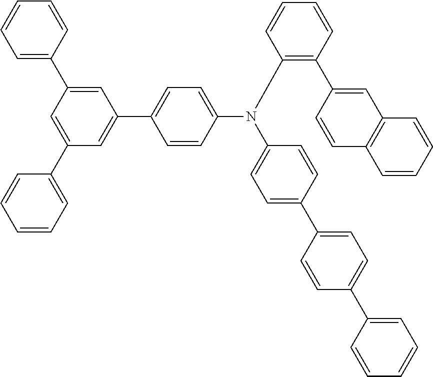 [SMILES: c1ccc(-c2ccc(-c3ccc(N(c4ccc(-c5cc(-c6ccccc6)cc(-c6ccccc6)c5)cc4)c4ccccc4-c4ccc5ccccc5c4)cc3)cc2)cc1] | 751.97 | 336 |
| Compound 12 | | |
| 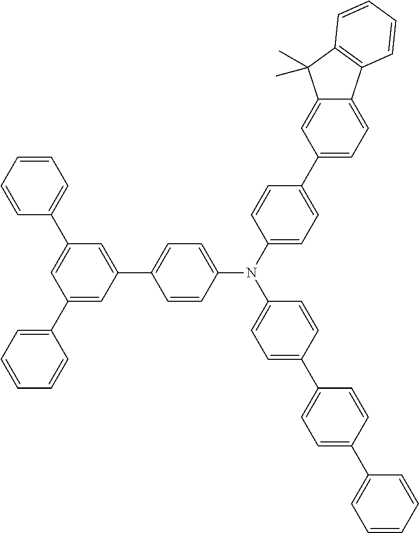 [SMILES: CC1(C)c2ccccc2-c2ccc(-c3ccc(N(c4ccc(-c5ccc(-c6ccccc6)cc5)cc4)c4ccc(-c5cc(-c6ccccc6)cc(-c6ccccc6)c5)cc4)cc3)cc21] | 818.08 | 367 |
| Compound 23 | | |
| 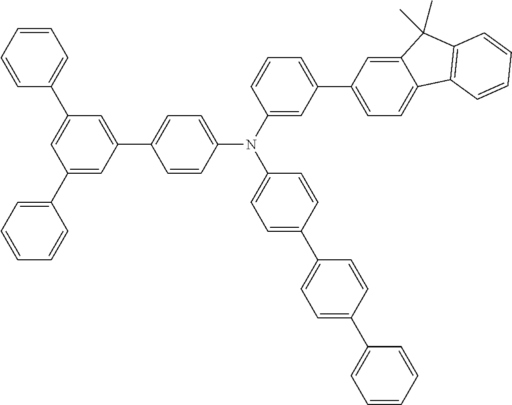 [SMILES: CC1(C)c2ccccc2-c2ccc(-c3cccc(N(c4ccc(-c5ccc(-c6ccccc6)cc5)cc4)c4ccc(-c5cc(-c6ccccc6)cc(-c6ccccc6)c5)cc4)c3)cc21] | 818.08 | 360 |
| Compound 30 | | |
| 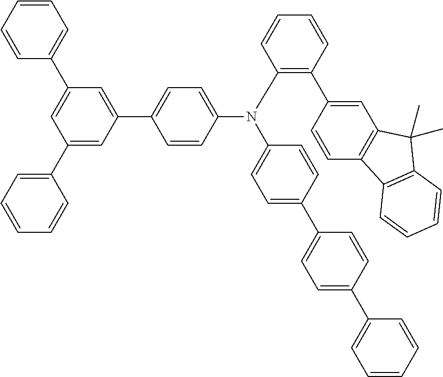 [SMILES: CC1(C)c2ccccc2-c2ccc(-c3ccccc3N(c3ccc(-c4ccc(-c5ccccc5)cc4)cc3)c3ccc(-c4cc(-c5ccccc5)cc(-c5ccccc5)c4)cc3)cc21] | 818.08 | 365 |
| Compound 34 | | |
| 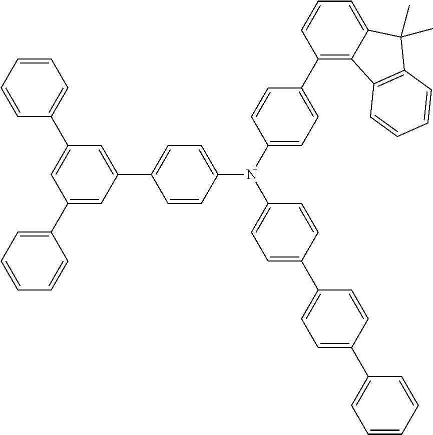 [SMILES: CC1(C)c2ccccc2-c2c(-c3ccc(N(c4ccc(-c5ccc(-c6ccccc6)cc5)cc4)c4ccc(-c5cc(-c6ccccc6)cc(-c6ccccc6)c5)cc4)cc3)cccc21] | 818.08 | 370 |
| Compound 21 | | |
| 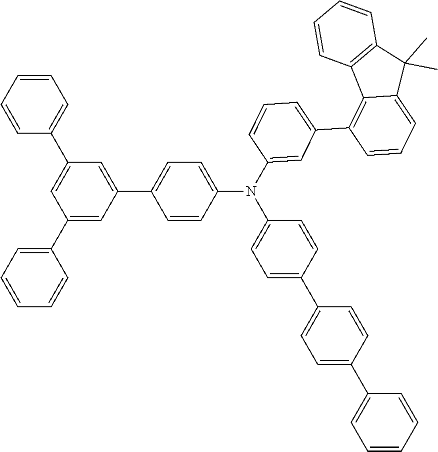 [SMILES: CC1(C)c2ccccc2-c2c(-c3cccc(N(c4ccc(-c5ccc(-c6ccccc6)cc5)cc4)c4ccc(-c5cc(-c6ccccc6)cc(-c6ccccc6)c5)cc4)c3)cccc21] | 818.08 | 361 |
| Compound 28 | | |
| 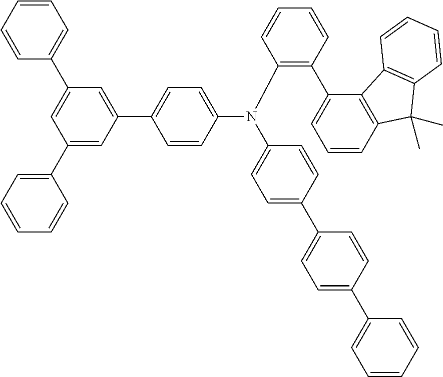 [SMILES: CC1(C)c2ccccc2-c2c(-c3ccccc3N(c3ccc(-c4ccc(-c5ccccc5)cc4)cc3)c3ccc(-c4cc(-c5ccccc5)cc(-c5ccccc5)c4)cc3)cccc21] | 818.08 | 366 |
| Compound 32 | | |
| 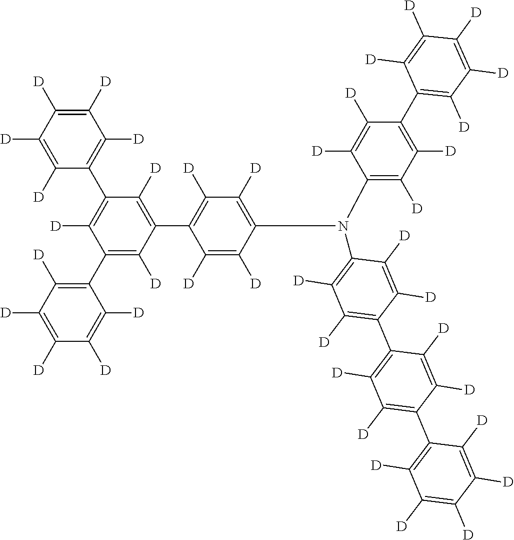 [SMILES: [2H]c1c([2H])c([2H])c(-c2c([2H])c([2H])c(-c3c([2H])c([2H])c(N(c4c([2H])c([2H])c(-c5c([2H])c([2H])c([2H])c([2H])c5[2H])c([2H])c4[2H])c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c([2H])c3[2H])c([2H])c2[2H])c([2H])c1[2H]] | 741.15 | 305 |
| Compound 38 | | |
| 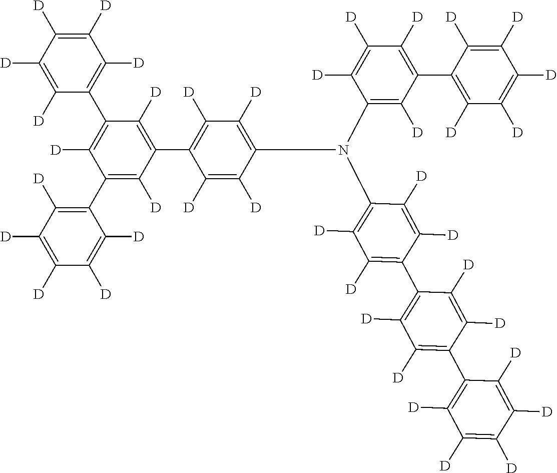 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c([2H])c([2H])c5[2H])c4[2H])c([2H])c3[2H])c([2H])c2[2H])c([2H])c1[2H]] | 741.15 | 296 |
| Compound 41 | | |
| 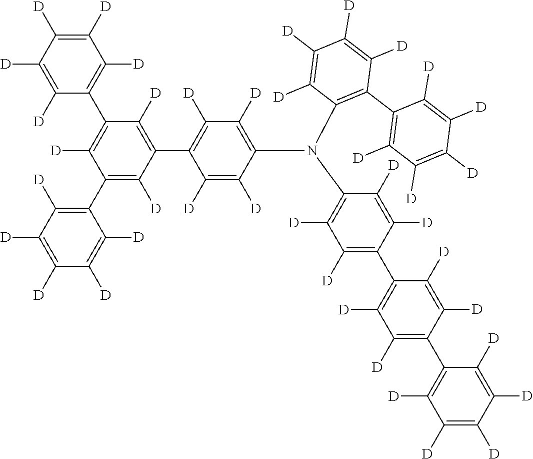 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c([2H])c4-c4c([2H])c([2H])c([2H])c([2H])c4[2H])c([2H])c3[2H])c([2H])c2[2H])c([2H])c1[2H]] | 741.15 | 300 |
| Compound 44 | | |
| 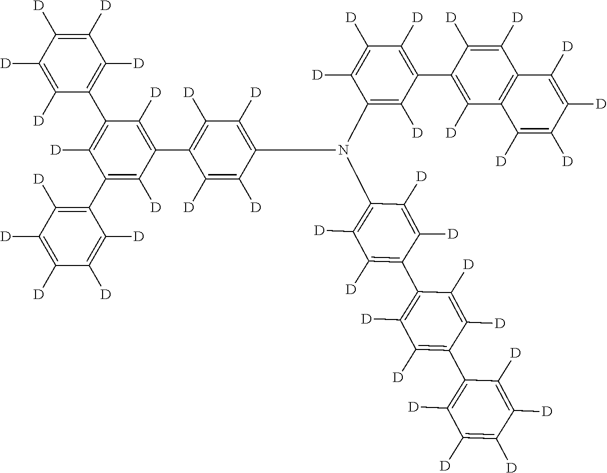 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | 793.22 | 335 |
| Compound 47 | | |
| 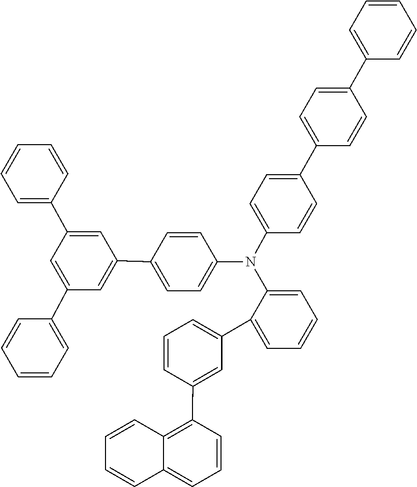 [SMILES: c1ccc(-c2ccc(-c3ccc(N(c4ccc(-c5cc(-c6ccccc6)cc(-c6ccccc6)c5)cc4)c4ccccc4-c4cccc(-c5cccc6ccccc56)c4)cc3)cc2)cc1] | 828.07 | 390 |
| Comparative Compound 4 | | |
| 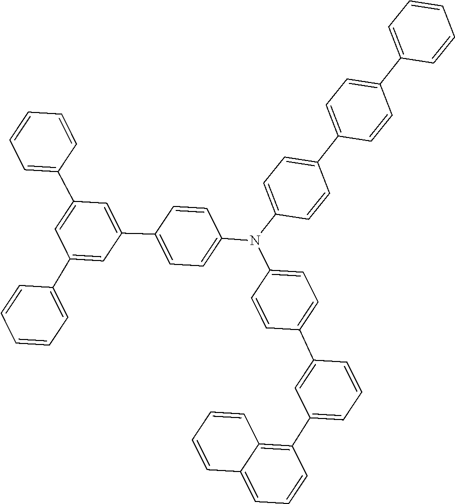 [SMILES: c1ccc(-c2ccc(-c3ccc(N(c4ccc(-c5cc(-c6ccccc6)cc(-c6ccccc6)c5)cc4)c4ccc(-c5cccc(-c6cccc7ccccc67)c5)cc4)cc3)cc2)cc1] | 828.07 | 392 |
| Comparative Compound 5 | | |

Since organic compounds used in organic light-emitting diodes generally form layers through a deposition method, it is required for such compounds to have an appropriate deposition temperature of approximately 290 to 370° C. When the deposition temperature exceeds 370° C., problems may arise such as reduced purity and instability of the formed layer.

Referring to Table 5, it was confirmed that the organic compounds represented by Chemical Formula A according to the present disclosure exhibit appropriate deposition temperatures, while for Comparative Compounds 4 and 5, the increased length of the linker connecting Ar to the nitrogen atom compared to Chemical Formula A leads to an increase in molecular weight, resulting in excessively high deposition temperatures.

While the embodiments of the present specification have been described in detail above, the present specification is not necessarily limited to these embodiments, and various modifications may be made without departing from the technical spirit of the present specification.

Therefore, the embodiments disclosed herein are intended to illustrate rather than limit the technical spirit of the present specification, and the scope of the technical spirit of the present specification is not limited by these embodiments. Accordingly, the embodiments described above should be understood as being illustrative in all respects and not restrictive.

While the preferred embodiments of the present disclosure have been described in detail above, the scope of the present disclosure is not limited thereto. Various modifications and improvements that may be made by those skilled in the art based on the basic concept of the present disclosure, as defined in the following claims, also fall within the scope of the present disclosure.

What is claimed is:

1. An organic compound represented by the following Chemical Formula A: 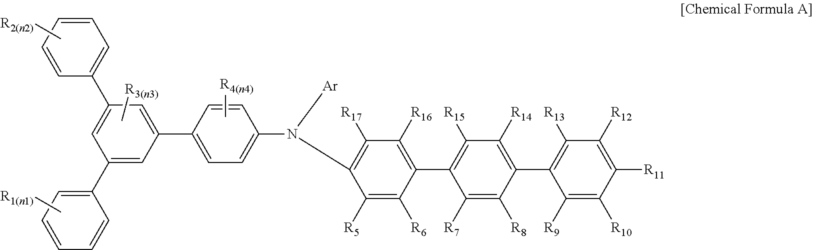
wherein in the chemical formula A,
Ar is one selected from the structures of M2, M3, M4, M11, M12, M13, M14, M15, M16, M17, M18, M19, M20, M21, M22, M23, M24, M25, M26, M27, M28, M29, M30 and M31, as shown below: 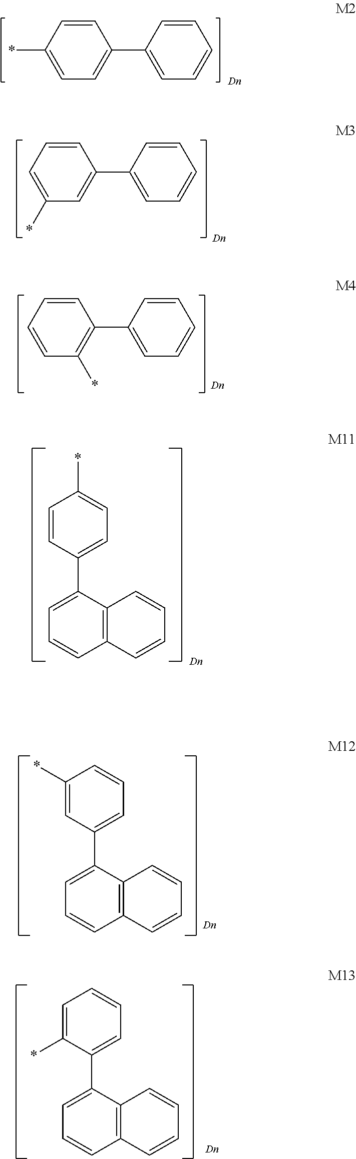 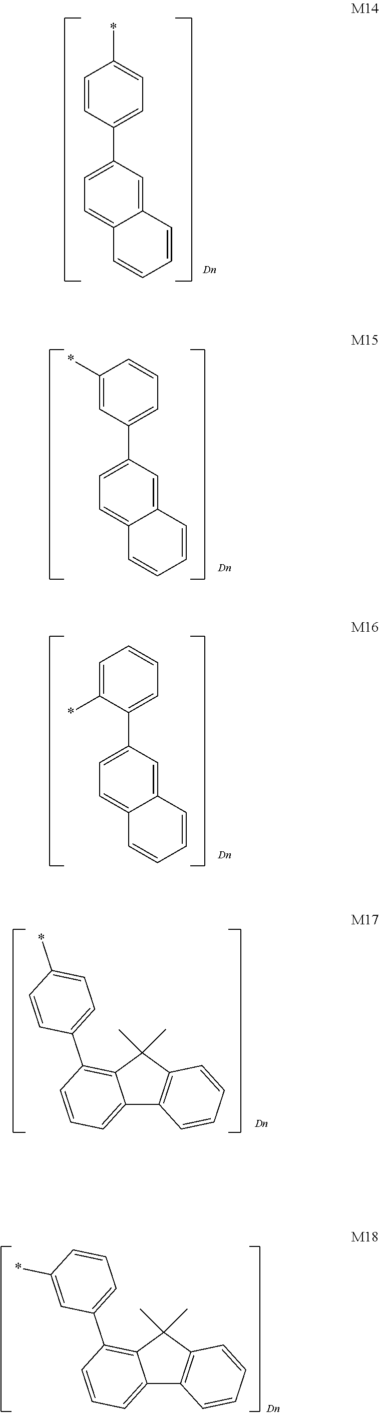 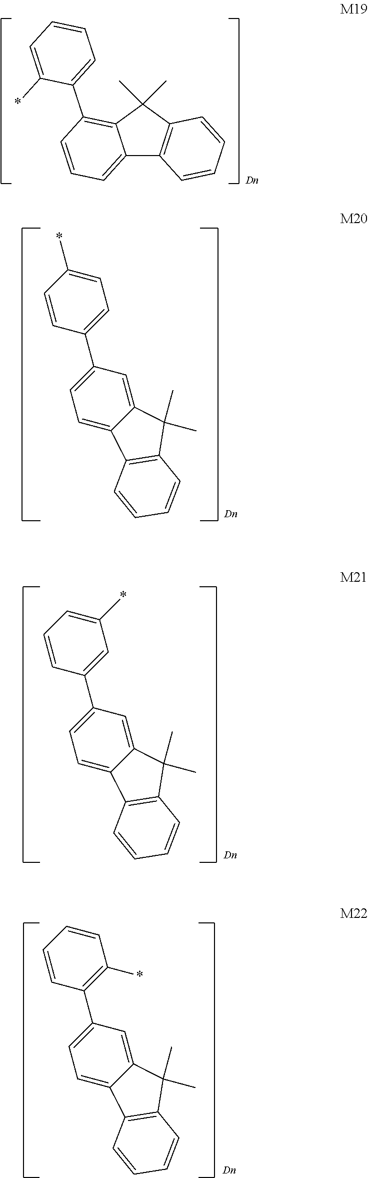 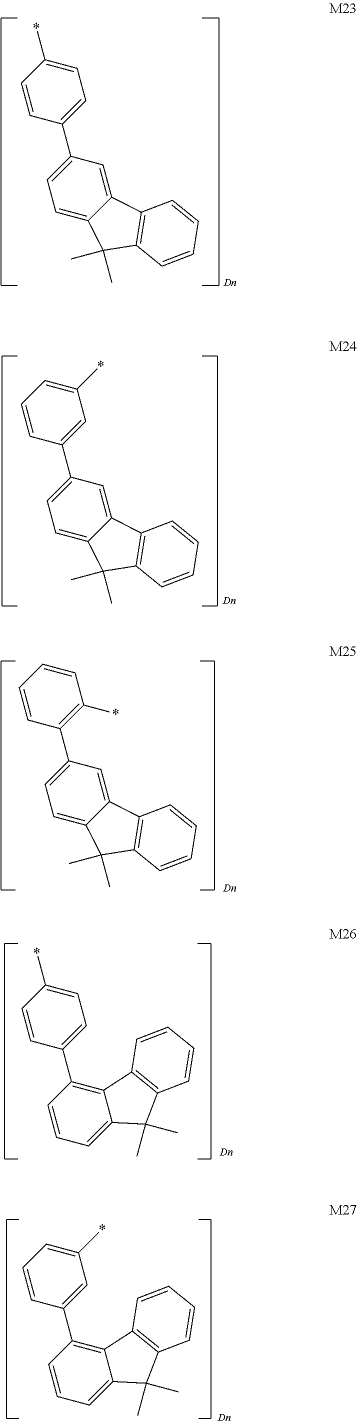 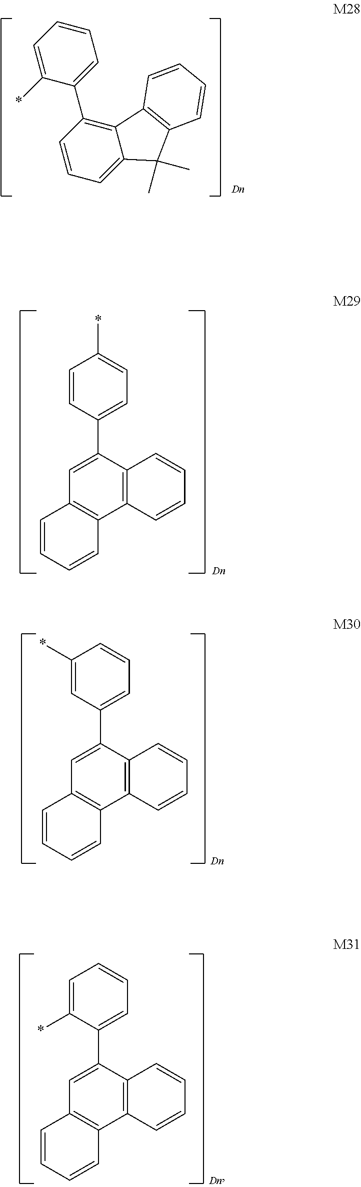
R₁ to R₁₇ are identical to or different from each other, and each of R₁ to R₁₇ independently represents one selected from the group consisting of hydrogen, deuterium, a substituted or unsubstituted alkyl group having 1 to 30 carbon atoms, a substituted or unsubstituted aryl group having 6 to 30 carbon atoms, and a substituted or unsubstituted heteroaryl group having 3 to 60 carbon atoms,
n1 and n2 are identical to or different from each other, and are each independently an integer from 0 to 5,
n3 is an integer from 0 to 3,
n4 is an integer from 0 to 4,
when R₁ to R₁₇ are substituted, the substituents are identical to or different from each other, and each substituent may be one or more selected from the group consisting of deuterium, a cyano group, a trifluoromethyl group, a nitro group, a halogen group, a hydroxy group, a trimethylsilyl (TMS) group, an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an arylalkyl group having 7 to 30 carbon atoms, a heteroaryl group having 3 to 60 carbon atoms, a heteroarylalkyl group having 4 to 60 carbon atoms, an amino group, an alkylamino group having 1 to 30 carbon atoms, an arylalkylamino group having 7 to 30 carbon atoms, an arylamino group having 6 to 30 carbon atoms, a heteroarylamino group having 3 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms; and when a plurality of substituents are present, the substituents are identical to or different from each other.

2. The organic compound of claim 1, wherein R₁ to R₁₇ are identical to or different from each other, and are each independently hydrogen or deuterium.

3. The organic compound of claim 1, wherein the organic compound represented by Chemical Formula A is selected from the group consisting of the following compounds:

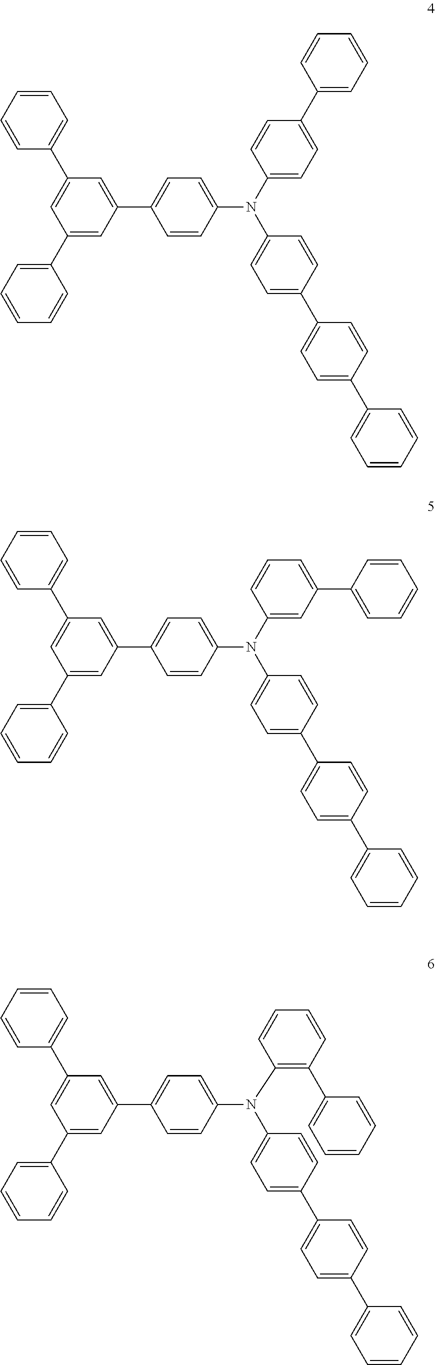 [SMILES: c1ccc(-c2ccc(-c3ccc(N(c4ccc(-c5ccccc5)cc4)c4ccc(-c5cc(-c6ccccc6)cc(-c6ccccc6)c5)cc4)cc3)cc2)cc1 ; c1ccc(-c2ccc(-c3ccc(N(c4ccc(-c5cc(-c6ccccc6)cc(-c6ccccc6)c5)cc4)c4cccc(-c5ccccc5)c4)cc3)cc2)cc1 ; c1ccc(-c2ccc(-c3ccc(N(c4ccc(-c5cc(-c6ccccc6)cc(-c6ccccc6)c5)cc4)c4ccccc4-c4ccccc4)cc3)cc2)cc1]

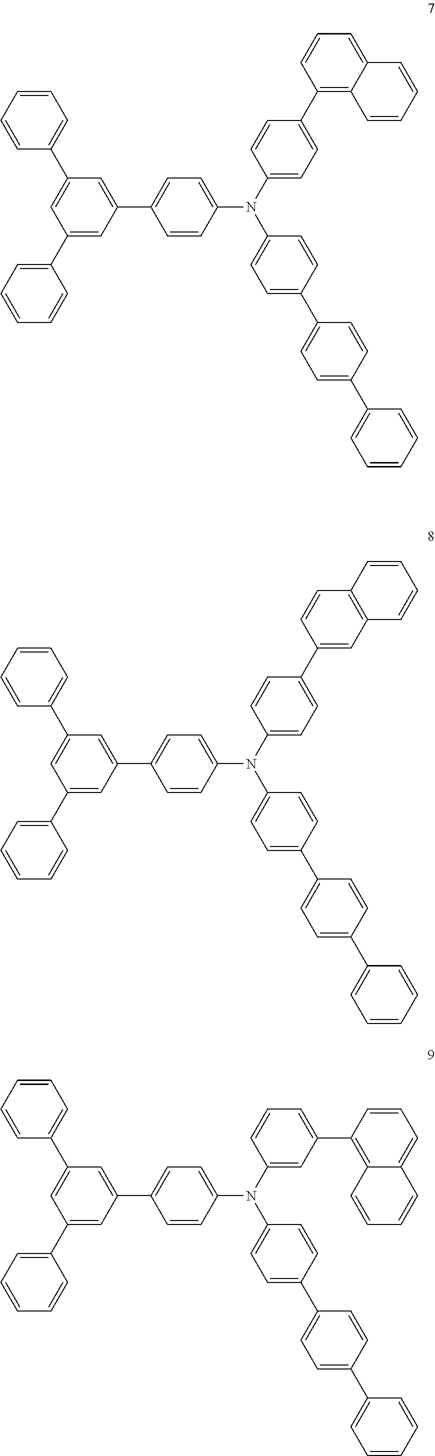 [SMILES: c1ccc(-c2ccc(-c3ccc(N(c4ccc(-c5cc(-c6ccccc6)cc(-c6ccccc6)c5)cc4)c4ccc(-c5cccc6ccccc56)cc4)cc3)cc2)cc1 ; c1ccc(-c2ccc(-c3ccc(N(c4ccc(-c5cc(-c6ccccc6)cc(-c6ccccc6)c5)cc4)c4ccc(-c5ccc6ccccc6c5)cc4)cc3)cc2)cc1 ; c1ccc(-c2ccc(-c3ccc(N(c4ccc(-c5cc(-c6ccccc6)cc(-c6ccccc6)c5)cc4)c4cccc(-c5cccc6ccccc56)c4)cc3)cc2)cc1]

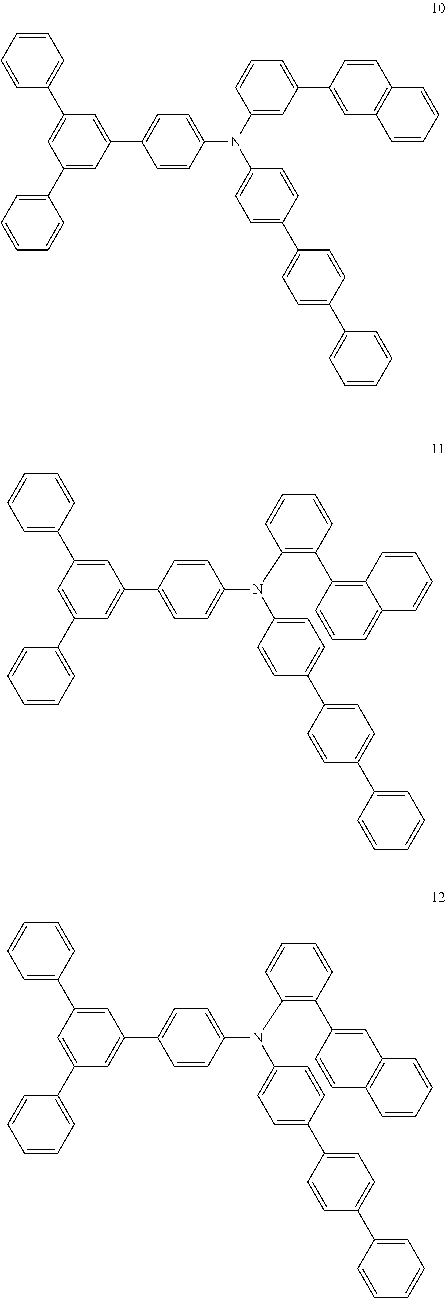 [SMILES: c1ccc(-c2ccc(-c3ccc(N(c4ccc(-c5cc(-c6ccccc6)cc(-c6ccccc6)c5)cc4)c4cccc(-c5ccc6ccccc6c5)c4)cc3)cc2)cc1 ; c1ccc(-c2ccc(-c3ccc(N(c4ccc(-c5cc(-c6ccccc6)cc(-c6ccccc6)c5)cc4)c4ccccc4-c4cccc5ccccc45)cc3)cc2)cc1 ; c1ccc(-c2ccc(-c3ccc(N(c4ccc(-c5cc(-c6ccccc6)cc(-c6ccccc6)c5)cc4)c4ccccc4-c4ccc5ccccc5c4)cc3)cc2)cc1]

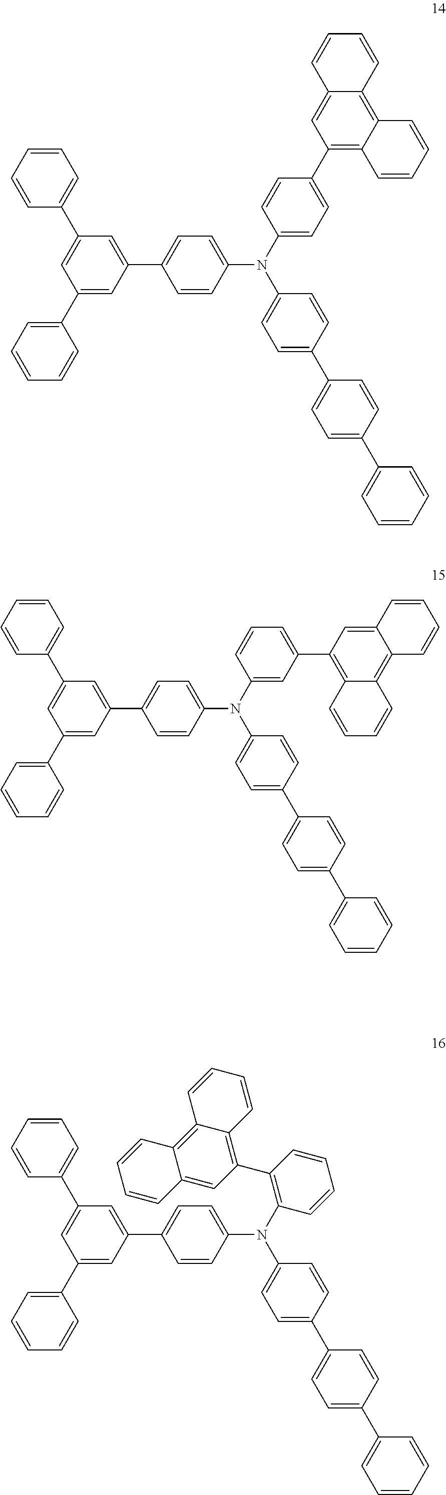 [SMILES: c1ccc(-c2ccc(-c3ccc(N(c4ccc(-c5cc(-c6ccccc6)cc(-c6ccccc6)c5)cc4)c4ccc(-c5cc6ccccc6c6ccccc56)cc4)cc3)cc2)cc1 ; c1ccc(-c2ccc(-c3ccc(N(c4ccc(-c5cc(-c6ccccc6)cc(-c6ccccc6)c5)cc4)c4cccc(-c5cc6ccccc6c6ccccc56)c4)cc3)cc2)cc1 ; c1ccc(-c2ccc(-c3ccc(N(c4ccc(-c5cc(-c6ccccc6)cc(-c6ccccc6)c5)cc4)c4ccccc4-c4cc5ccccc5c5ccccc45)cc3)cc2)cc1]

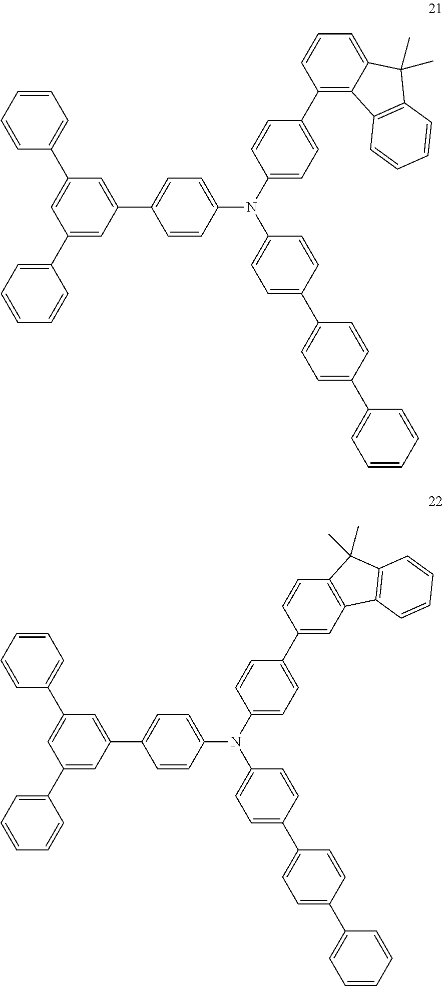 [SMILES: CC1(C)c2ccccc2-c2c(-c3ccc(N(c4ccc(-c5ccc(-c6ccccc6)cc5)cc4)c4ccc(-c5cc(-c6ccccc6)cc(-c6ccccc6)c5)cc4)cc3)cccc21 ; CC1(C)c2ccccc2-c2cc(-c3ccc(N(c4ccc(-c5ccc(-c6ccccc6)cc5)cc4)c4ccc(-c5cc(-c6ccccc6)cc(-c6ccccc6)c5)cc4)cc3)ccc21]

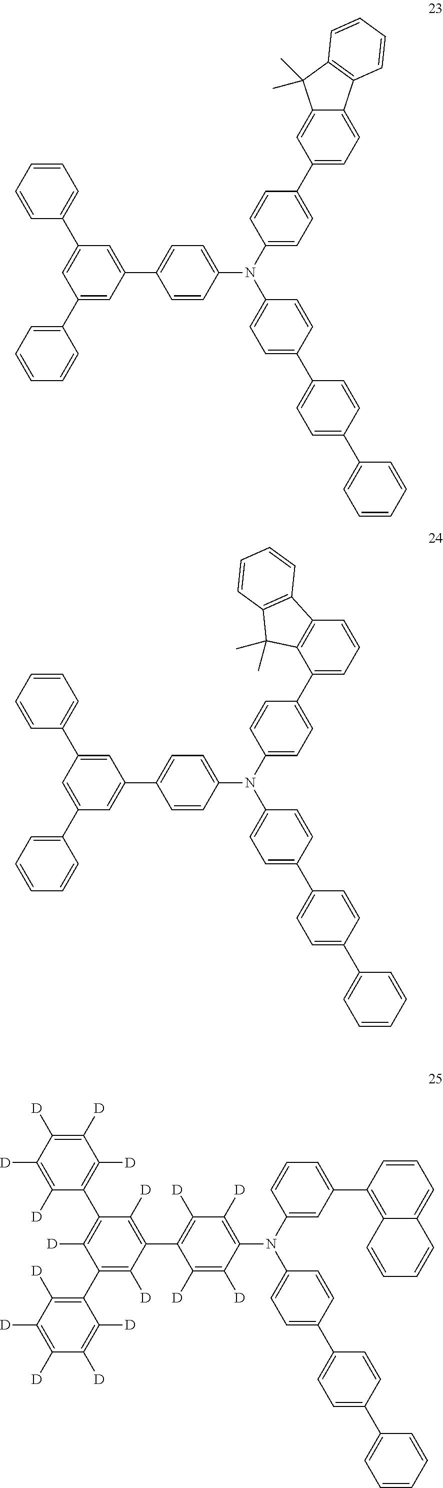 [SMILES: CC1(C)c2ccccc2-c2ccc(-c3ccc(N(c4ccc(-c5ccc(-c6ccccc6)cc5)cc4)c4ccc(-c5cc(-c6ccccc6)cc(-c6ccccc6)c5)cc4)cc3)cc21 ; CC1(C)c2ccccc2-c2cccc(-c3ccc(N(c4ccc(-c5ccc(-c6ccccc6)cc5)cc4)c4ccc(-c5cc(-c6ccccc6)cc(-c6ccccc6)c5)cc4)cc3)c21 ; [2H]c1c([2H])c([2H])c(-c2c([2H])c(-c3c([2H])c([2H])c([2H])c([2H])c3[2H])c([2H])c(-c3c([2H])c([2H])c(N(c4ccc(-c5ccc(-c6ccccc6)cc5)cc4)c4cccc(-c5cccc6ccccc56)c4)c([2H])c3[2H])c2[2H])c([2H])c1[2H]]

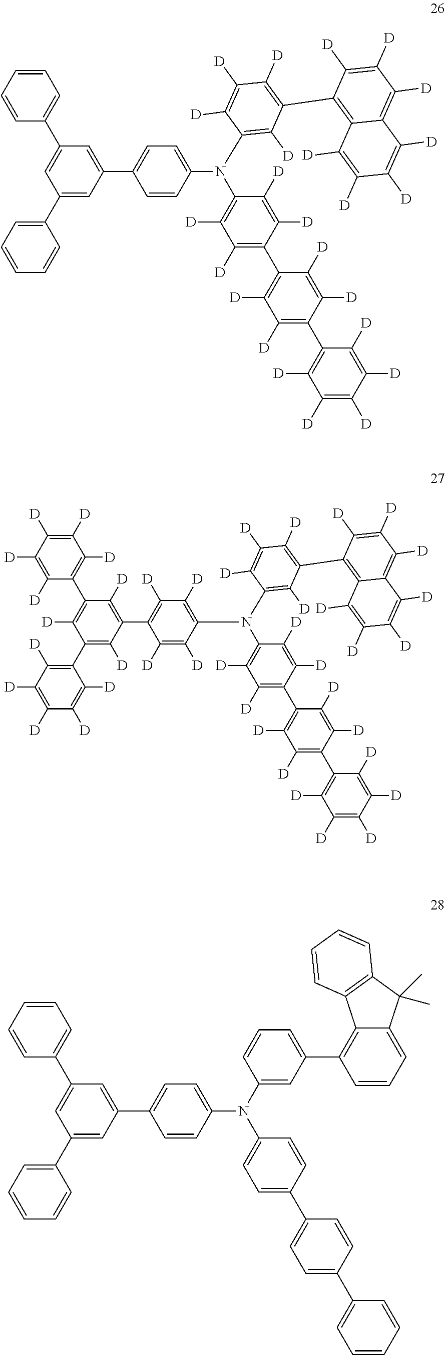

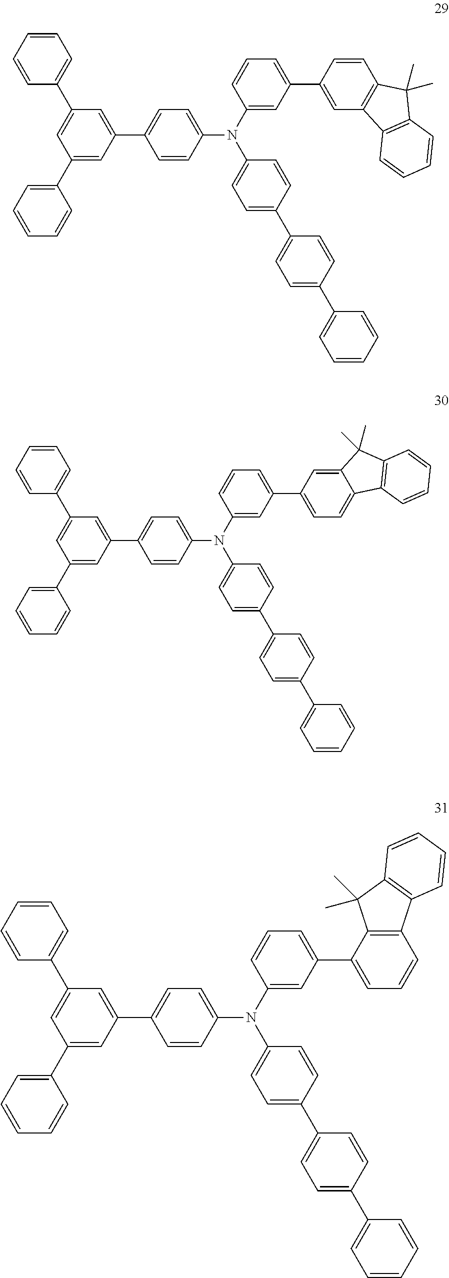 [SMILES: CC1(C)c2ccccc2-c2cc(-c3cccc(N(c4ccc(-c5ccc(-c6ccccc6)cc5)cc4)c4ccc(-c5cc(-c6ccccc6)cc(-c6ccccc6)c5)cc4)c3)ccc21 ; CC1(C)c2ccccc2-c2ccc(-c3cccc(N(c4ccc(-c5ccc(-c6ccccc6)cc5)cc4)c4ccc(-c5cc(-c6ccccc6)cc(-c6ccccc6)c5)cc4)c3)cc21 ; CC1(C)c2ccccc2-c2cccc(-c3cccc(N(c4ccc(-c5ccc(-c6ccccc6)cc5)cc4)c4ccc(-c5cc(-c6ccccc6)cc(-c6ccccc6)c5)cc4)c3)c21]

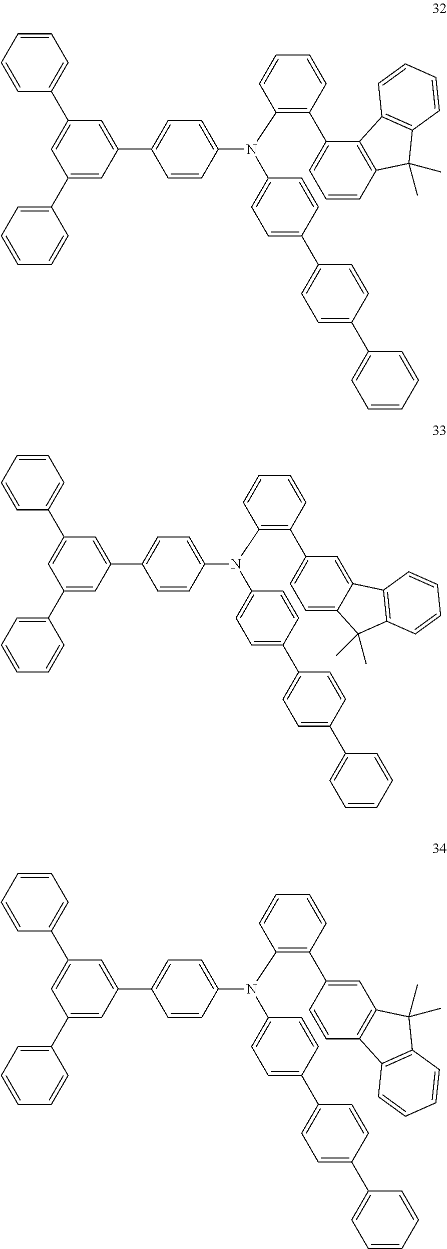 [SMILES: CC1(C)c2ccccc2-c2c(-c3ccccc3N(c3ccc(-c4ccc(-c5ccccc5)cc4)cc3)c3ccc(-c4cc(-c5ccccc5)cc(-c5ccccc5)c4)cc3)cccc21 ; CC1(C)c2ccccc2-c2cc(-c3ccccc3N(c3ccc(-c4ccc(-c5ccccc5)cc4)cc3)c3ccc(-c4cc(-c5ccccc5)cc(-c5ccccc5)c4)cc3)ccc21 ; CC1(C)c2ccccc2-c2ccc(-c3ccccc3N(c3ccc(-c4ccc(-c5ccccc5)cc4)cc3)c3ccc(-c4cc(-c5ccccc5)cc(-c5ccccc5)c4)cc3)cc21]

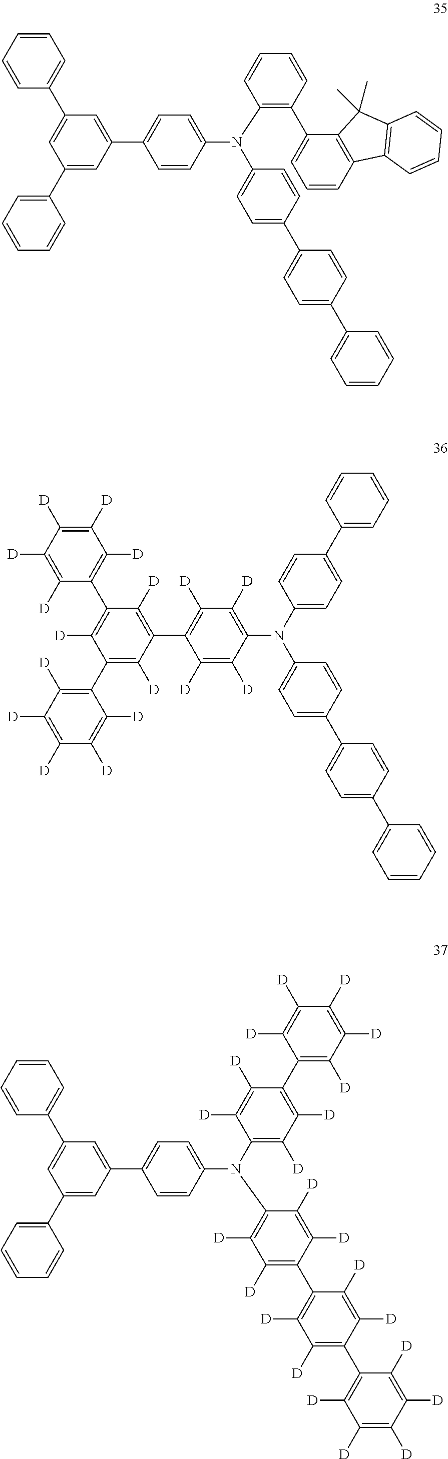

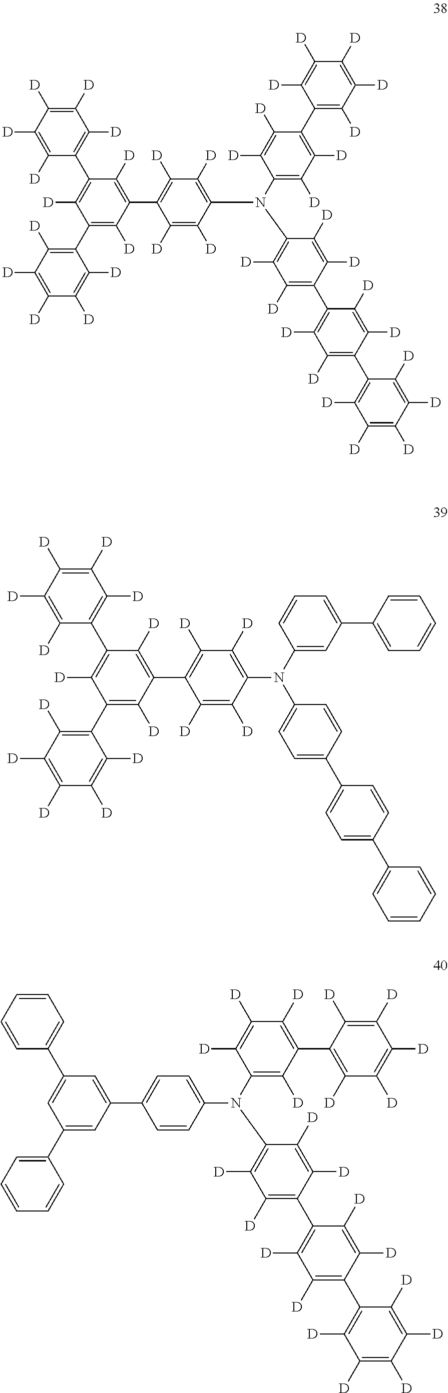

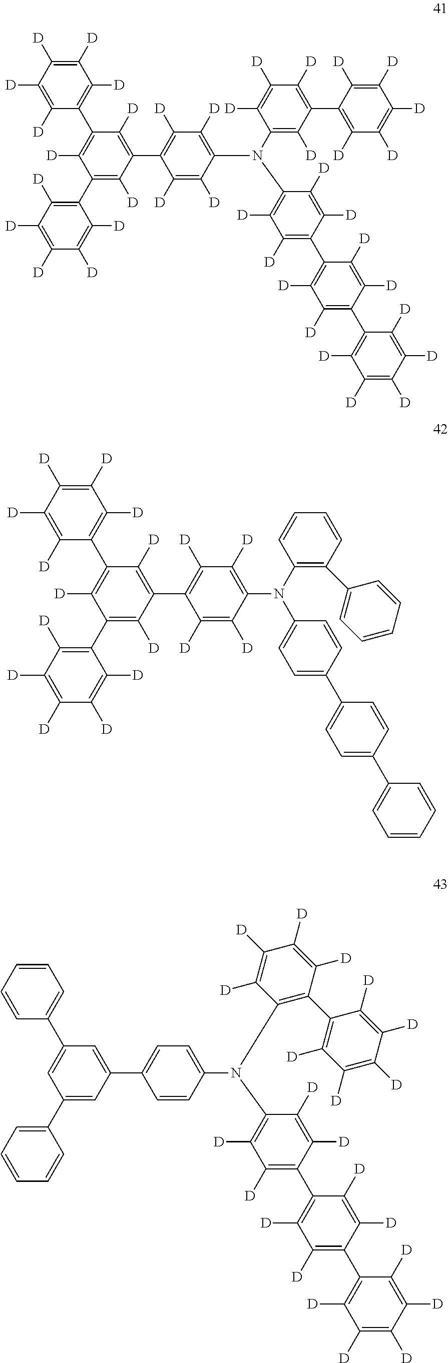

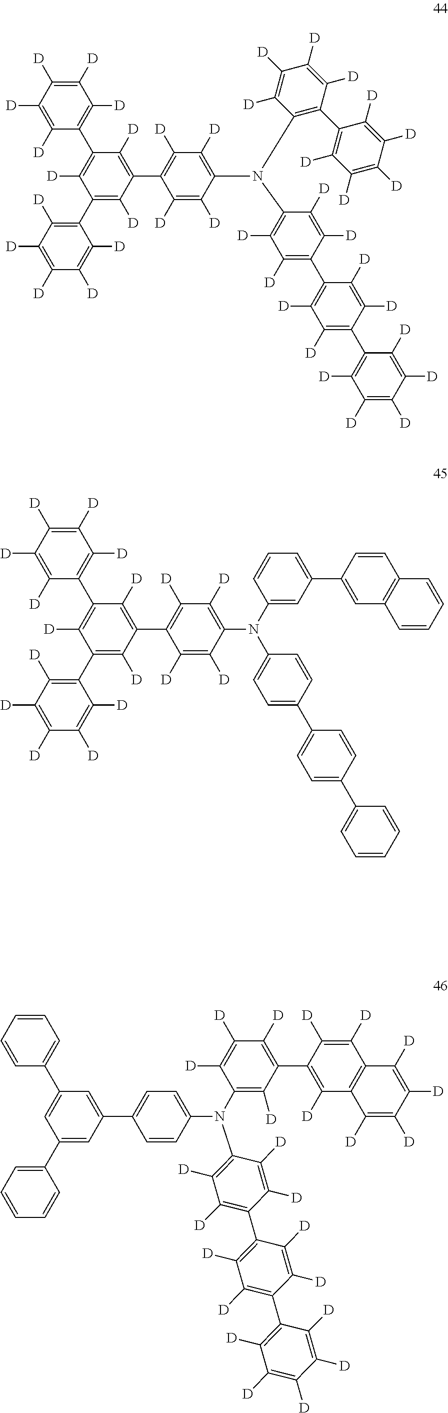

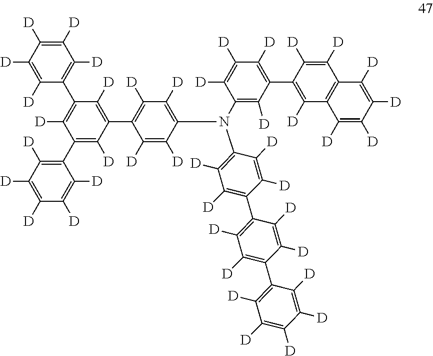 [SMILES: [2H]c1c([2H])c([2H])c(-c2c([2H])c([2H])c(-c3c([2H])c([2H])c(N(c4c([2H])c([2H])c(-c5c([2H])c(-c6c([2H])c([2H])c([2H])c([2H])c6[2H])c([2H])c(-c6c([2H])c([2H])c([2H])c([2H])c6[2H])c5[2H])c([2H])c4[2H])c4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]]

4. An organic light-emitting diode comprising:
a first electrode;
a second electrode facing the first electrode; and
one or more organic layers disposed between the first electrode and the second electrode,
wherein at least one of the organic layers includes the organic compound represented by Chemical Formula A according to claim 1.

5. The organic light-emitting diode of claim 4, wherein the organic layer including the organic compound represented by Chemical Formula A is a hole transport layer or a hole transport auxiliary layer.
